(19) United States
(12) Patent Application Publication
(10) Pub. No.: US 2024/0100177 A1
(43) Pub. Date: Mar. 28, 2024

(54) ANTIBODY-OLIGONUCLEOTIDE COMPLEXES AND USES THEREOF

(71) Applicant: Dyne Therapeutics, Inc., Waltham, MA (US)

(72) Inventors: Scott Hilderbrand, Cambridge, MA (US); John Najim, Waltham, MA (US); Qifeng Qiu, Waltham, MA (US); Benjamin Vieira, Waltham, MA (US); Timothy Weeden, Waltham, MA (US); Sean Spring, Waltham, MA (US)

(73) Assignee: Dyne Therapeutics, Inc., Waltham, MA (US)

(21) Appl. No.: 18/265,065

(22) PCT Filed: Dec. 3, 2021
(86) PCT No.: PCT/US2021/061748

§ 371 Date: Jun. 2, 2023

Related U.S. Application Data
(60) Provisional application No. 63/121,573, filed on Dec. 4, 2020.

(51) Int. Cl. A61K 47/68

(52) U.S. Cl. CPC A61K 47/6807; A61K 47/6889; A61K 47/6849

(57) ABSTRACT

Provided herein are oligonucleotide-antibody complexes, methods of preparing the complexes, and methods of using the complexes (e.g., treating muscle diseases). In particular, provided are compounds of Formula (I) and various methods for the preparation of the compounds. Also provided are pharmaceutical compositions comprising compounds of Formula (I) and methods of treating muscle disease in a subject by administering a compound or composition described herein.

### RELATED APPLICATION

This application claims priority under 35 U.S.C. § 119(e) to U.S. Provisional Application, U.S. Ser. No. 63/121,573, filed Dec. 4, 2020, which is incorporated herein by reference in its entirety.

### BACKGROUND

Complexes comprising tissue- or cell-specific proteins (e.g., antibodies) covalently linked to therapeutic drugs (e.g., small molecules or oligonucleotides) offer excellent opportunities for delivery of said therapeutic drugs. Therefore, there is a need for the design and preparation of effective complexes.

### SUMMARY

In one aspect, the disclosure provides compounds of Formula (I), and pharmaceutically acceptable salts, solvates, hydrates, polymorphs, co-crystals, tautomers, stereoisomers, isotopically labeled derivatives, prodrugs, and pharmaceutical compositions thereof. The compounds are useful for the treatment of a muscle disease in a subject in need thereof.

In one aspect, provided herein are methods of preparing a compound of Formula (I):

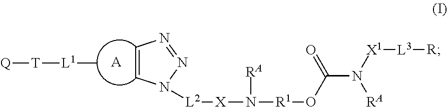

or a salt thereof, the method comprising coupling a targeting agent (Q) with a compound of Formula (II):

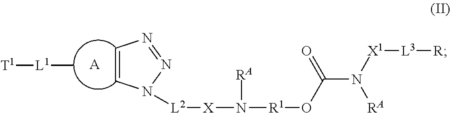

or a salt thereof, to provide a compound of Formula (I), wherein:
T is

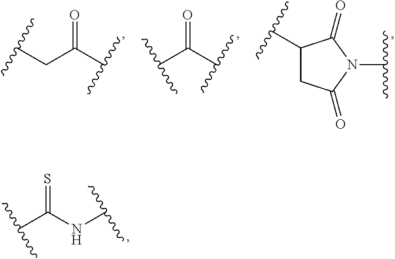

or —S—;
T¹ is

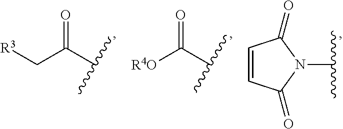

S═C═N—, or

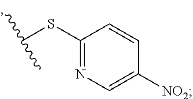 [SMILES: *Sc1ccc([N+](=O)[O-])cn1]

R³ is a leaving group (e.g., halogen, tosylate, mesylate, or triflate);
R⁴ is hydrogen; substituted or unsubstituted alkyl; substituted or unsubstituted heterocyclyl; substituted or unsubstituted aryl; substituted or unsubstituted heteroaryl; or an oxygen protecting group;
L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
A is substituted or unsubstituted carbocycle or substituted or unsubstituted heterocycle, or A is absent and L¹ is bonded directly to the triazole ring;
L² is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
X is a cleavable moiety;
R¹ is substituted or unsubstituted arylene, or substituted or unsubstituted alkylene, or a combination thereof;
X¹ is a bond or a peptide;
L³ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
each R^{A} is independently hydrogen or substituted or unsubstituted alkyl; and
R is a molecular payload.

In another aspect, provided herein are methods of preparing compounds of Formula (I):

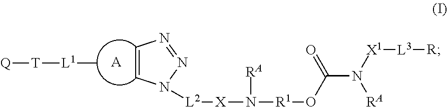

or a salt thereof, the method comprising reacting a compound of Formula (IV):

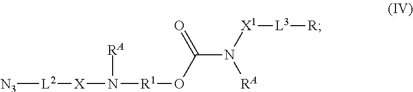

or a salt thereof, with a compound of Formula (B):

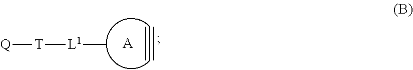

or a salt thereof, to provide a compound of Formula (I), wherein:
Q is targeting agent;
T is

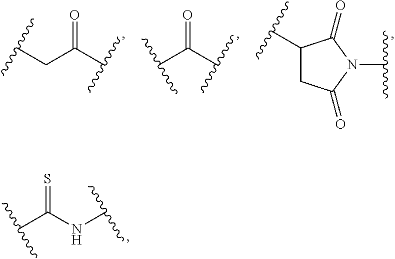

or —S—;
L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
A is substituted or unsubstituted carbocycle or substituted or unsubstituted heterocycle, or A is absent and L¹ is bonded directly to the triazole ring;
A¹ is substituted or unsubstituted carbocycle or substituted or unsubstituted heterocycle, or A¹ is absent and L¹ is bonded directly to one terminus of the alkyne;
L² is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
X is a cleavable moiety;
R¹ is substituted or unsubstituted arylene, or substituted or unsubstituted alkylene, or a combination thereof;
X¹ is a bond or a peptide;
L³ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
each R^{A} is independently hydrogen or substituted or unsubstituted alkyl; and
R is a molecular payload.

In another aspect, provided herein are methods of preparing compounds of Formula (I):

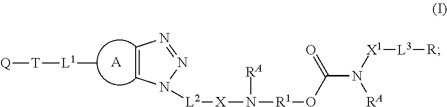

or a salt thereof, the method comprising reacting a targeting agent (Q); a compound of Formula (IV):

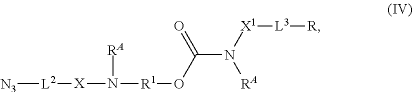

or a salt thereof; and a compound of Formula (III):

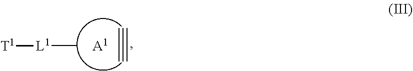

or a salt thereof; to provide a compound of Formula (I), or a salt thereof, wherein:
T is

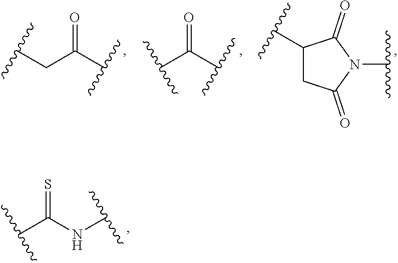

or —S—;
T¹ is

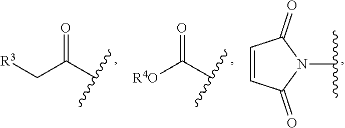

S═C═N—, or

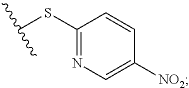 [SMILES: *Sc1ccc([N+](=O)[O-])cn1]

R³ is a leaving group (e.g., halogen, tosylate, mesylate, or triflate);
R⁴ is hydrogen; substituted or unsubstituted alkyl; substituted or unsubstituted heterocyclyl; substituted or unsubstituted aryl; substituted or unsubstituted heteroaryl; or an oxygen protecting group;
L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
A is substituted or unsubstituted carbocycle or substituted or unsubstituted heterocycle, or A is absent and L¹ is bonded directly to the triazole ring;
A¹ is substituted or unsubstituted carbocycle or substituted or unsubstituted heterocycle, or A¹ is absent and L¹ is bonded directly to one terminus of the alkyne;
L² is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
X is a cleavable moiety;
R¹ is substituted or unsubstituted arylene, or substituted or unsubstituted alkylene, or a combination thereof;
X¹ is a bond or a peptide;
L³ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
each R^{A} is independently hydrogen or substituted or unsubstituted alkyl; and
R is a molecular payload.

In another aspect, provided herein are compounds of Formula (I):

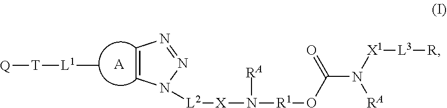

or a pharmaceutically acceptable salt, tautomer, stereoisomer, or isotopically enriched derivative thereof, wherein:
Q is a targeting agent;
T is

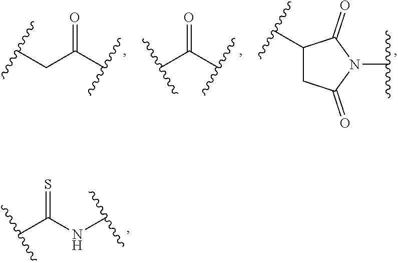

or —S—;
L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
A is substituted or unsubstituted carbocycle or substituted or unsubstituted heterocycle, or A is absent and L¹ is bonded directly to the triazole ring;
L² is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
X is a cleavable moiety;
R¹ is substituted or unsubstituted arylene, or substituted or unsubstituted alkylene, or a combination thereof;
X¹ is a bond or a peptide;
L³ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; each R^{A} is independently hydrogen or substituted or unsubstituted alkyl; and
R is a molecular payload.

In certain embodiments, Q is an antibody; and R is an oligonucleotide. In certain embodiments, Q is an anti-TfR antibody; and R is a phosphorodiamidate morpholino oligomer (PMO).

The details of certain embodiments of the disclosure are set forth in the Detailed Description of Certain Embodiments, as described below. Other features, objects, and advantages of the disclosure will be apparent from the Definitions, Examples, Figures, and Claims.

### BRIEF DESCRIPTION OF THE DRAWINGS

FIG. 1 shows the ¹H NMR spectrum of Compound 8.

FIG. 2 shows the ¹H NMR spectrum of Compound B.

FIG. 3 shows the mass spectrum of Complex 1 after papain digestion to remove the molecular payload.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by appropriate methods, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The disclosure additionally encompasses compounds as individual isomers substantially free of other isomers, and alternatively, as mixtures of various isomers.

In a formula, [symbol] is a single bond where the stereochemistry of the moieties immediately attached thereto is not specified, --- is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and 1 or more heteroatoms within the parent chain (“heteroC₁₋₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

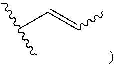

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), bicyclo[6.1.0]non-4-enyl (C₉), bicyclo[6.1.0]nonanyl (C₉), bicyclo[6.1.0]non-4-ynyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}—, —ON(R^{bb})₂, —N(R^{bb})₂, —N(R^{bb})₃⁺X⁻, —N(OR^{cc})R^{bb}, —SH, —SR^{aa}—, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}—, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}—, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{aa})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{f} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{f} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R—, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R—, —NR^{bb}CO₂R—, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes (—CHO), esters (e.g., —CO₂R^{aa}, —C(═O)SR^{aa}, —C(═S)SR^{aa}), amides (e.g., —C(═O)N(R^{bb})₂, —C(═O)NR^{bb}SO₂R^{aa}, —C(═S)N(R^{bb})₂), and imines (e.g., —C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}—, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined herein.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

The term “leaving group” is given its ordinary meaning in the art of synthetic organic chemistry and refers to an atom or a group capable of being displaced by a nucleophile. See, for example, Smith, March's Advanced Organic Chemistry 6th ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties. Further exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein).

In certain embodiments, “leaving group” is represented by “LG” in chemical formulae. In certain embodiments, the leaving group is —O-succinimide. In certain embodiments, LG is —O-succinimide. In certain embodiments, the leaving group is triflate. In certain embodiments, LG is triflate. In certain embodiments, the leaving group is trifluoroacetate. In certain embodiments, LG is trifluoroacetate.

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and claims. The disclosure is not intended to be limited in any manner by the above exemplary listing of substituents.

### Other Definitions

The following definitions are more general terms used throughout the present application.

The term “antibody” refers to a polypeptide that includes at least one immunoglobulin variable domain or at least one antigenic determinant, e.g., paratope that specifically binds to an antigen. In some embodiments, an antibody is a full-length antibody, e.g., a full-length IgG. In some embodiments, an antibody is a chimeric antibody. In some embodiments, an antibody is a humanized antibody. However, in some embodiments, an antibody is a Fab′ fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Example linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

The term “charge-neutral oligonucleotide” refers to oligonucleotide analogs comprising charge neutral backbones. Examples of charge neutral oligonucleotides include, without limitation, phosphorodiamidate morpholino oligomers (PMOs) and peptide nucleic acids (PNA), e.g., as described in Jarver et al., (Nucleic Acid Therapeutics, Vol. 25, No. 2, 2015), incorporated herein by reference.

The term “charged oligonucleotide” refers to an oligonucleotide analog comprising a backbone that has a net negative or net positive charge at a physiological pH (e.g., pH 7.35-pH 7.45). In some embodiments, a charged oligonucleotide has a net negative charge at a physiological pH (referred to herein as negatively charged oligonucleotide). In some embodiments, a charged oligonucleotide has a net positive charge at a physiological pH (referred to herein as positively charged oligonucleotide). In some embodiments, a charged oligonucleotide comprises a phosphodiester backbone that has a net negative charge at physiological pH. In some embodiments, a charged oligonucleotide comprises a phosphothioate backbone that has a net negative charge at physiological pH. Examples of charged oligonucleotides include, without limitation, RNAi oligonucleotides (e.g., siRNAs) and gapmers. In certain embodiments, the charged oligonucleotide is a gapmer. In certain embodiments, the charged oligonucleotide is an siRNA.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions, such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·xH₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇₋₁₂ arylalkyl esters of the compounds described herein may be preferred.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a

The term “targeting agent” refers to a member of a specific binding pair, i.e., a member of a pair of molecules, wherein one of the pair of molecules has an area on its surface, or a cavity that specifically binds to, and is, therefore, defined as complementary with a particular spatial and polar organization of the other molecule, so that the pair have the property of binding specifically to each other. Examples of types of specific binding pairs are antigen-antibody, biotin-avidin, hormone-hormone receptor, receptor-ligand, enzyme-substrate, and IgG-protein A.

The term “cleavable moiety” refers to a divalent moiety which is capable of being separated (e.g., detached, split, disconnected, hydrolyzed, a stable bond within the moiety is broken) into distinct entities. In embodiments, a cleavable moiety is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents).

As used herein, the term “molecular payload” refers to a molecule or species that functions to modulate a biological outcome. In some embodiments, a molecular payload is linked to, or otherwise associated with a muscle-targeting agent. In some embodiments, the molecular payload is a small molecule, a protein, a peptide, a nucleic acid, or an oligonucleotide. In some embodiments, the molecular payload is a hydrophobic small molecule. In some embodiments, the molecular payload is a charge-neutral oligonucleotide (e.g., a phosphorodiamidate morpholino oligomer). In some embodiments, the molecular payload functions to modulate the transcription of a DNA sequence, to modulate the expression of a protein, or to modulate the activity of a protein. In some embodiments, the molecular payload is an oligonucleotide, e.g., an oligonucleotide that comprises a strand having a region of complementarity to a target gene.

As used herein, the term “muscle disease gene” refers to a gene having a least one disease allele correlated with and/or directly or indirectly contributing to, or causing, a muscle disease. In some embodiments, the muscle disease is a rare disease, e.g., as defined by the Genetic and Rare Diseases Information Center (GARD), which is a program of the National Center for Advancing Translational Sciences (NCATS). In some embodiments, the muscle disease is a rare disease that is characterized as affecting fewer than 200,000 people. In some embodiments, the muscle disease is a single-gene disease. In some embodiments, a muscle disease gene is a gene listed in Table 1.

As used herein, the term, “muscle-targeting agent,” refers to a molecule that specifically binds to an antigen expressed on muscle cells. The antigen in or on muscle cells may be a membrane protein, for example an integral membrane protein or a peripheral membrane protein. Typically, a muscle-targeting agent specifically binds to an antigen on muscle cells that facilitates internalization of the muscle-targeting agent (and any associated molecular payload) into the muscle cells. In some embodiments, a muscle-targeting agent specifically binds to an internalizing, cell surface receptor on muscles and is capable of being internalized into muscle cells through receptor mediated internalization. In some embodiments, the muscle-targeting agent is a small molecule, a protein, a peptide, a nucleic acid (e.g., an aptamer), or an antibody. In some embodiments, the muscle-targeting agent is linked to a molecular payload. In some embodiments, the muscle-targeting agent is a muscle targeting protein (e.g., an antibody)

As used herein, the term, “muscle-targeting antibody,” refers to a muscle-targeting agent that is an antibody that specifically binds to an antigen found in or on muscle cells. In some embodiments, a muscle-targeting antibody specifically binds to an antigen on muscle cells that facilitates internalization of the muscle-targeting antibody (and any associated molecular payment) into the muscle cells. In some embodiments, the muscle-targeting antibody specifically binds to an internalizing, cell surface receptor present on muscle cells. In some embodiments, the muscle-targeting antibody is an antibody that specifically binds to a transferrin receptor.

As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidite morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleotides (e.g. 2′-O-methyl sugar modifications, purine or pyrimidine modifications). In some embodiments, an oligonucleotide may comprise one or more modified internucleotide linkage. In some embodiments, an oligonucleotide may comprise one or more phosphorothioate linkages, which may be in the Rp or Sp stereochemical conformation.

As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

As used herein, the term, “transferrin receptor” (also known as TFRC, CD71, p90, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

As used herein, the term “drug-to-antibody ratio (DAR)” refers to the number of drugs conjugated to the antibodies. This DAR number can vary with the nature of the antibody and of the drug used along with the experimental conditions used for the conjugation (ratio of antibody and molecular payload in the starting reaction material, the reaction time, the nature of the solvent and of the cosolvent if any). The DAR that is determined is a mean value. One example of a method that can be used to determine the DAR is described in Dimitrov et al., 2009, Therapeutic Antibodies and Protocols, vol 525, 445, Springer Science, incorporated herein by reference.

### DETAILED DESCRIPTION OF CERTAIN EMBODIMENTS

Provided herein are antibody-oligonucleotide complexes, methods of preparing the conjugates, and methods of using the complexes. In one aspect, the disclosure provides compounds of Formula (I), and pharmaceutically acceptable salts, solvates, hydrates, polymorphs, co-crystals, tautomers, stereoisomers, isotopically labeled derivatives, prodrugs, and pharmaceutical compositions thereof. The compounds are useful for the treatment of a muscle disease in a subject in need thereof.

### I. Compounds

In one aspect, disclosed is a compound of Formula (I):

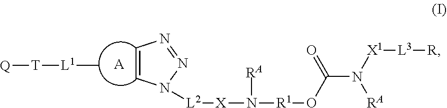

or a pharmaceutically acceptable salt, tautomer, stereoisomer, or isotopically enriched derivative thereof, wherein:
Q is a targeting agent;
T is

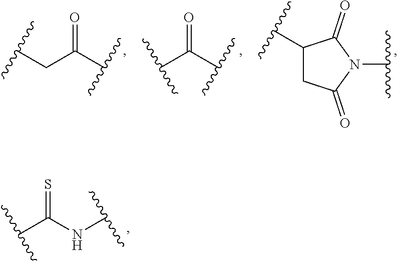

or —S—;
L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
A is substituted or unsubstituted carbocycle or substituted or unsubstituted heterocycle, or A is absent and L¹ is bonded directly to the triazole ring;
L² is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
X is a cleavable moiety;
R¹ is substituted or unsubstituted arylene, or substituted or unsubstituted alkylene, or a combination thereof;
X¹ is a bond or a peptide;
L³ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
each R^{A} is independently hydrogen or substituted or unsubstituted alkyl; and
R is a molecular payload.

### Group T

As described herein T is:

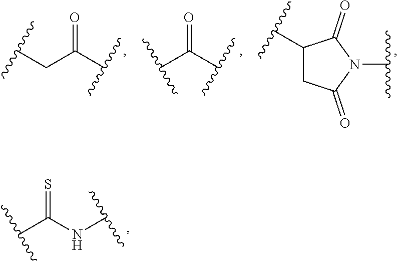

### or —S—.

In certain embodiments, T is:

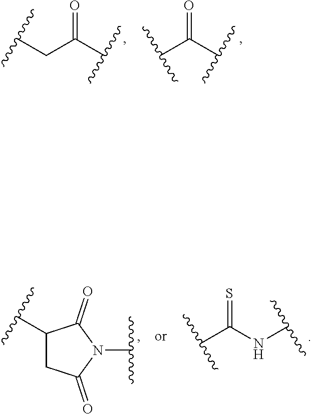

In certain embodiments, T is:

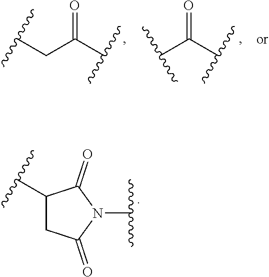

In certain embodiments, T is:

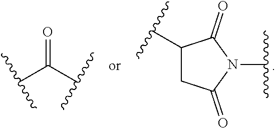

In certain embodiments, T is:

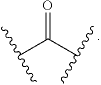 [SMILES: *C(*)=O]

In certain embodiments, T is:

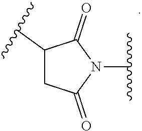 [SMILES: *C1CC(=O)N(*)C1=O]

### Group L¹

As described herein, L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof. In embodiments where L¹ is a combination of any of the alternatives listed in its definition, L¹ may include more than one occurrence of any listed alternative.

In certain embodiments, L¹ is —C(═O)—, —O—, —NR^{A}—, substituted or unsubstituted alkylene, substituted or unsubstituted heteroalkylene, substituted or unsubstituted carbocyclylene, substituted or unsubstituted heterocyclylene, substituted or unsubstituted heteroarylene, or substituted or unsubstituted arylene, or a combination thereof.

In certain embodiments, L¹ is —C(═O)—, —O—, —NR^{A}—, substituted or unsubstituted alkylene, or substituted or unsubstituted heteroalkylene, or a combination thereof.

In certain embodiments, L¹ is —C(═O)—, —NR^{A}C(═O)O—, substituted or unsubstituted alkylene, or substituted or unsubstituted heteroalkylene, or a combination thereof.

In certain embodiments, L¹ is —C(═O)—, —NR^{A}C(═O)O—, substituted or unsubstituted C₁₋₂₀ alkylene, or substituted or unsubstituted C₁₋₃₀ heteroalkylene, or a combination thereof. In certain embodiments, L¹ is —C(═O)—, —NR^{A}C(═O)O—, substituted or unsubstituted C₁₋₁₀ alkylene, substituted or unsubstituted C₁₋₂₀ heteroalkylene, or a combination thereof. In certain embodiments, L¹ is —C(═O)—, —NR^{A}C(═O)O—, substituted or unsubstituted C₁₋₆ alkylene, substituted or unsubstituted C₁₋₁₅ heteroalkylene, or a combination thereof.

In certain embodiments, L¹ is a combination of —C(═O)—, —NR^{A}C(═O)O—, unsubstituted C₁₋₂₀ alkylene, and unsubstituted C₁₋₃₀ heteroalkylene. In certain embodiments, L¹ is a combination of —C(═O)—, —NR^{A}C(═O)O—, unsubstituted C₁₋₁₀ alkylene, and unsubstituted C₁₋₂₀ heteroalkylene. In certain embodiments, L¹ is a combination of —C(═O)—, —NR^{A}C(═O)O—, unsubstituted C₁₋₆ alkylene, and unsubstituted C₁₋₁₅ heteroalkylene.

In certain embodiments, L¹ is —C(═O)—,

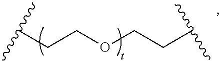

—NR^{A}C(═O)O—, or substituted or unsubstituted C₁₋₆ alkylene, or a combination thereof; wherein t is 1-12.

In certain embodiments, L¹ is

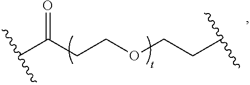

—NR^{A}C(═O)O—, or substituted or unsubstituted C₁₋₆ alkylene, or a combination thereof; wherein t is 1-12.

In certain embodiments, L¹ is

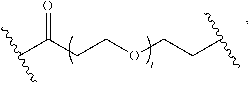

or —NR^{A}C(═O)O—CH₃—, or a combination thereof; wherein t is 1-12.

In certain embodiments, L¹ is a combination of —C(═O)—,

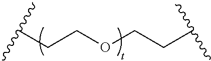

—NR^{A}C(═O)O—, and substituted or unsubstituted C₁₋₆ alkylene; wherein t is 1-12.

In certain embodiments, L¹ is a combination of

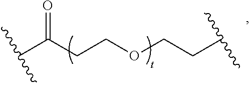

—NR^{A}C(═O)O—, and substituted or unsubstituted C₁₋₆ alkylene; wherein t is 1-12.

In certain embodiments, L¹ is a combination of

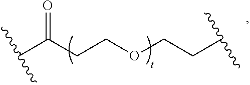

and —NR^{A}C(═O)O—CH₃—; wherein t is 1-12.

In certain embodiments, L¹ is

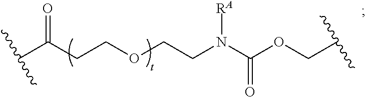

wherein t is 1-12. In certain embodiments, t is 1-10, 1-8, 1-6, 1-5, 1-4, 1-3, or 1-2. In certain embodiments, t is 12, 11, 10, 9, 8, 7, 6, 5, 4, 3, 2, or 1. In certain embodiments, t is 3 or 4. In certain embodiments, t is 3. In certain embodiments, t is 4.

In certain embodiments, L¹ is

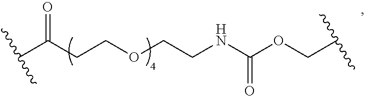

wherein t is 1-12. In certain embodiments, t is 1-10, 1-8, 1-6, 1-5, 1-4, 1-3, or 1-2. In certain embodiments, t is 12, 11, 10, 9, 8, 7, 6, 5, 4, 3, 2, or 1. In certain embodiments, t is 3 or 4. In certain embodiments, t is 3. In certain embodiments, t is 4.

In certain embodiments, L¹ is

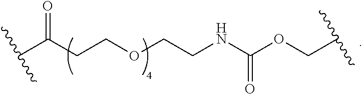

In certain embodiments, L¹ is

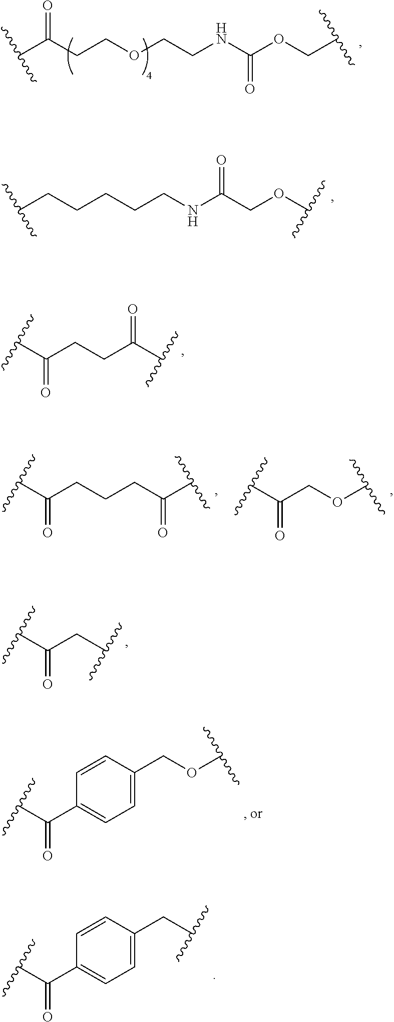

In certain embodiments, L¹ is

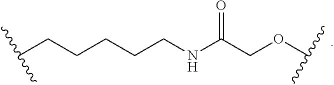 [SMILES: *CCCCCNC(=O)CO*]

In certain embodiments, L¹ is

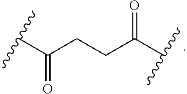 [SMILES: *C(=O)CCC(*)=O]

In certain embodiments, L¹ is

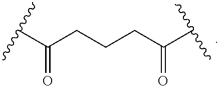 [SMILES: *C(=O)CCCC(*)=O]

In certain embodiments, L¹ is

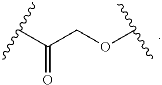 [SMILES: *OCC(*)=O]

In certain embodiments, L¹ is

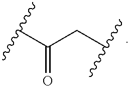 [SMILES: *CC(*)=O]

In certain embodiments, L¹ is

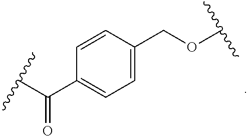 [SMILES: *OCc1ccc(C(*)=O)cc1]

In certain embodiments, L¹ is

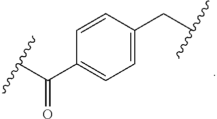 [SMILES: *Cc1ccc(C(*)=O)cc1]

### Group A

As described herein, A is substituted or unsubstituted carbocycle or substituted or unsubstituted heterocycle, or A is absent and L¹ is bonded directly to the triazole ring.

In certain embodiments, A is substituted or unsubstituted carbocycle, or substituted or unsubstituted heterocycle. In certain embodiments, A is absent and L¹ is bonded directly to the triazole ring.

In certain embodiments, A is substituted or unsubstituted heterocycle. In certain embodiments, A is substituted or unsubstituted 5-8 membered heterocycle. In certain embodiments, A is substituted or unsubstituted 6-8 membered heterocycle. In certain embodiments, A is substituted or unsubstituted 7-8 membered heterocycle. In certain embodiments, A is a substituted or unsubstituted 8-membered heterocycle. In certain embodiments, A is a substituted 8-membered heterocycle. In certain embodiments, A is a substituted or unsubstituted hexahydroazocine. In certain embodiments, A is a substituted or unsubstituted tetrahydroazocine. In certain embodiments, A is a substituted or unsubstituted dihydroazocine.

In certain embodiments, A is:

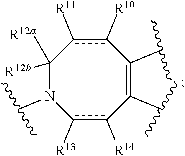

wherein:
[symbol] is a single or double bond;
R¹⁰ is hydrogen, halogen, alkoxy, substituted or unsubstituted alkyl, or substituted or unsubstituted aryl;
R¹¹ is hydrogen, halogen, alkoxy, substituted or unsubstituted alkyl, or substituted or unsubstituted aryl; or R¹⁰ and R¹¹ together with the atoms to which they are attached form a substituted or unsubstituted aryl;
R^{12a} and R^{12b} are each hydrogen or together with the carbon to which they are attached form a carbonyl;
R¹³ is hydrogen, halogen, alkoxy, substituted or unsubstituted alkyl, or substituted or unsubstituted aryl; and
R¹⁴ is hydrogen, halogen, alkoxy, substituted or unsubstituted alkyl, or substituted or unsubstituted aryl; or R¹³ and R¹⁴ together with the atoms to which they are attached form a substituted or unsubstituted aryl.

In certain embodiments, A is:

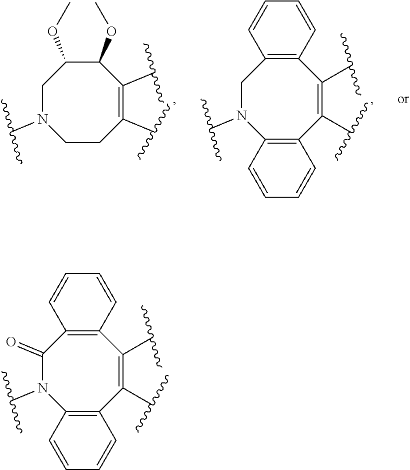 [SMILES: */C1=C(\*)[C@H](OC)[C@@H](OC)CN(*)CC1 ; */C1=C(\*)c2ccccc2N(*)Cc2ccccc21 ; */C1=C(\*)c2ccccc2N(*)C(=O)c2ccccc21]

In certain embodiments, A is:

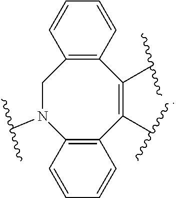 [SMILES: */C1=C(\*)c2ccccc2N(*)Cc2ccccc21]

In certain embodiments, A is substituted or unsubstituted carbocycle. In certain embodiments, A is substituted or unsubstituted C₅₋₁₀ carbocycle. In certain embodiments, A is substituted or unsubstituted C₈₋₁₀ carbocycle. In certain embodiments, A is substituted or unsubstituted C₈₋₉ carbocycle. In certain embodiments, A is a substituted or unsubstituted C₈ carbocycle. In certain embodiments, A is a substituted or unsubstituted cyclooctene.

In certain embodiments, A is:

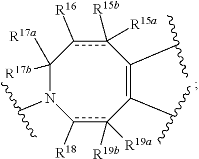

wherein:
[symbol] is a single or double bond;
R^{15a} and R^{15b} are each independently hydrogen, halogen, alkoxy, substituted or unsubstituted alkyl, or substituted or unsubstituted aryl;
R¹⁶ is hydrogen, halogen, alkoxy, substituted or unsubstituted alkyl, or substituted or unsubstituted aryl; or one of R^{15a}/R^{15b} and R¹⁶ together with the atoms to which they are attached form a substituted or unsubstituted aryl;
R^{17a} and R^{17b} are each hydrogen or together with the carbon to which they are attached form a carbonyl;
R¹⁸ is hydrogen, halogen, alkoxy, substituted or unsubstituted alkyl, or substituted or unsubstituted aryl; and
R^{19a} and R^{19b} are each independently hydrogen, halogen, alkoxy, substituted or unsubstituted alkyl, or substituted or unsubstituted aryl.

In certain embodiments, A is:

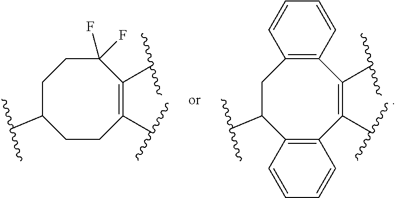 [SMILES: */C1=C(\*)C(F)(F)CCC(*)CC1 ; */C1=C(\*)c2ccccc2C(*)Cc2ccccc21]

In certain embodiments, A is:

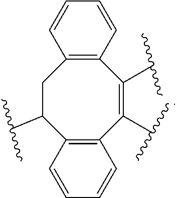 [SMILES: */C1=C(\*)c2ccccc2C(*)Cc2ccccc21]

In certain embodiments, A is:

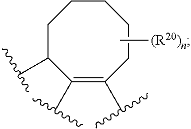

wherein:
each R²⁰ is independently halogen, alkoxy, substituted or unsubstituted alkyl, or substituted or unsubstituted aryl; or two occurrences of R²⁰ together with the atoms to which they are attached form a substituted or unsubstituted aryl or carbocyclic ring; and
n is 0-8.

In certain embodiments, A is:

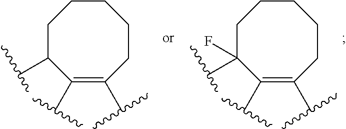 [SMILES: */C1=C(\*)C(*)CCCCC1 ; */C1=C(\*)C(*)(F)CCCCC1]

In certain embodiments, A is a substituted or unsubstituted C₉ carbocycle. In certain embodiments, A is a substituted or unsubstituted bicyclic fused C₉ carbocycle. In certain embodiments, A is a substituted or unsubstituted bicyclic fused C₉ cycloalkyl or cycloalkenyl. In certain embodiments, A is a substituted or unsubstituted bicyclo[6.1.0]non-4-enyl. In certain embodiments, A is an unsubstituted bicyclo[6.1.0]non-4-enyl.

In certain embodiments, A is:

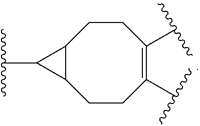 [SMILES: */C1=C(\*)CCC2C(*)C2CC1]

In certain embodiments, A is:

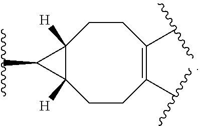 [SMILES: */C1=C(\*)CC[C@H]2[C@H](*)[C@H]2CC1]

In certain embodiments, A is:

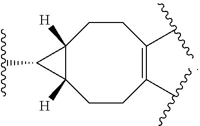 [SMILES: */C1=C(\*)CC[C@H]2[C@@H](*)[C@H]2CC1]

### Group L²

As described herein, L² is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof. In embodiments where L² is a combination of any of the alternatives listed in its definition, L² may include more than one occurrence of any listed alternative.

In certain embodiments, L² is —C(═O)—, —O—, —NR^{A}—, substituted or unsubstituted alkylene, substituted or unsubstituted heteroalkylene, substituted or unsubstituted carbocyclylene, substituted or unsubstituted heterocyclylene, substituted or unsubstituted heteroarylene, or substituted or unsubstituted arylene, or a combination thereof.

In certain embodiments, L² is —C(═O)—, —O—, —NR^{A}—, substituted or unsubstituted alkylene, or substituted or unsubstituted heteroalkylene, or a combination thereof.

In certain embodiments, L² is —C(═O)—, substituted or unsubstituted alkylene, or substituted or unsubstituted heteroalkylene, or a combination thereof.

In certain embodiments, L² is —C(═O)—, or substituted or unsubstituted heteroalkylene, or a combination thereof.

In certain embodiments, L² is —C(═O)—, or substituted or unsubstituted C₁₋₃₀ heteroalkylene, or a combination thereof. In certain embodiments, L² is —C(═O)—, substituted or unsubstituted C₁₋₂₀ heteroalkylene, or a combination thereof. In certain embodiments, L² is —C(═O)—, substituted or unsubstituted C₁₋₁₅ heteroalkylene, or a combination thereof. In certain embodiments, L² is —C(═O)—, substituted or unsubstituted C₁₋₁₂ heteroalkylene, or a combination thereof.

In certain embodiments, L² is a combination of —C(═O)— and unsubstituted C₁₋₃₀ heteroalkylene. In certain embodiments, L² is a combination of —C(═O)— and unsubstituted C₁₋₂₀ heteroalkylene. In certain embodiments, L² is a combination of —C(═O)— and unsubstituted C₁₋₁₅ heteroalkylene. In certain embodiments, L² is a combination of —C(═O)— and unsubstituted C₁₋₁₂ heteroalkylene.

In certain embodiments, L² is —C(═O)—, or

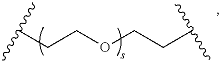

or a combination thereof; wherein s is 1-12.

In certain embodiments, L² is

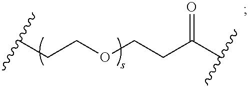

wherein s is 1-12. In certain embodiments, s is 1-10, 1-8, 1-6, 1-5, 1-4, 1-3, or 1-2. In certain embodiments, s is 12, 11, 10, 9, 8, 7, 6, 5, 4, 3, 2, or 1. In certain embodiments, s is 3 or 4. In certain embodiments, s is 3. In certain embodiments, s is 4.

In certain embodiments, L² is

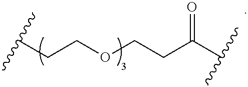

### Group X

As described herein, X is a cleavable moiety. The term “cleavable moiety” refers to a divalent moiety which is capable of being separated (e.g., detached, split, disconnected, hydrolyzed, a stable bond within the moiety is broken) into distinct entities. In embodiments, a cleavable moiety is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). In certain embodiments, X is cleavable by a protease.

In certain embodiments, X is a bond or a peptide. In certain embodiments, X is a peptide. In certain embodiments, X is a peptide comprising 2-10 amino acids. In certain embodiments, the peptide is linear. In certain embodiments, the peptide is branched. In certain embodiments, X is a peptide comprising at least one valine. In certain embodiments, X is a peptide comprising at least one citrulline. In certain embodiments, X is a peptide comprising at least one valine and at least one citrulline.

In certain embodiments, X is —ZₘYₘ—; wherein each occurrence of Z is independently an amino acid, each occurrence of Y is independently an amino acid, and each m is independently 1, 2, or 3. In certain embodiments, X is —Zₘ—Yₘ—; wherein at least one of Z and Y is a valine. In certain embodiments, X is —ZₘYₘ—; wherein at least one of Z and Y is a citrulline. In certain embodiments, Z is valine. In certain embodiments, Y is citrulline. In certain embodiments, Z is valine, and Y is citrulline. In certain embodiments, each m is 1. In certain embodiments, Z is valine, Y is citrulline, and each m is 1.

In certain embodiments X is:

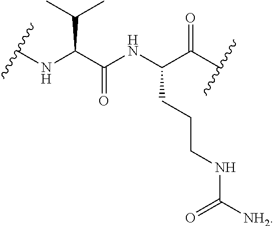 [SMILES: *N[C@H](C(=O)N[C@@H](CCCNC(N)=O)C(*)=O)C(C)C]

In certain embodiments, —Zₘ—Yₘ— is:

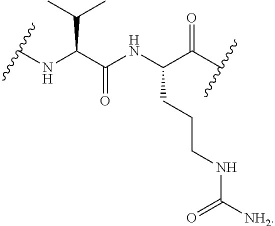 [SMILES: *N[C@H](C(=O)N[C@@H](CCCNC(N)=O)C(*)=O)C(C)C]

### Group R¹

As described herein, R¹ is substituted or unsubstituted arylene, or substituted or unsubstituted alkylene, or a combination thereof. In embodiments where RV is a combination of the alternatives listed in its definition, R¹ may include more than one occurrence of any listed alternative.

In certain embodiments, R¹ is substituted or unsubstituted phenylene, or substituted or unsubstituted alkylene, or a combination thereof. In certain embodiments, R¹ is substituted or unsubstituted phenylene, or substituted or unsubstituted C₁₋₆ alkylene, or a combination thereof. In certain embodiments, R¹ is substituted or unsubstituted phenylene, or substituted or unsubstituted C₁₋₄ alkylene, or a combination thereof. In certain embodiments, R¹ is substituted or unsubstituted phenylene, or substituted or unsubstituted C₁₋₂ alkylene, or a combination thereof. In certain embodiments, R¹ is substituted or unsubstituted phenylene, or substituted or unsubstituted methylene, or a combination thereof. In certain embodiments, R¹ is unsubstituted phenylene, or unsubstituted methylene, or a combination thereof.

In certain embodiments, R¹ is a combination of substituted or unsubstituted phenylene and substituted or unsubstituted alkylene. In certain embodiments, R¹ is a combination of substituted or unsubstituted phenylene and substituted or unsubstituted C₁₋₆ alkylene. In certain embodiments, R¹ is a combination of substituted or unsubstituted phenylene and substituted or unsubstituted C₁₋₄ alkylene. In certain embodiments, R¹ is substituted or unsubstituted phenylene and substituted or unsubstituted C₁₋₂ alkylene. In certain embodiments, R¹ is a combination of unsubstituted phenylene and unsubstituted methylene.

In certain embodiments, R¹ is:

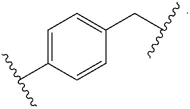 [SMILES: *Cc1ccc(*)cc1]

### Group L³

As described herein, L³ is a spacer group, wherein L³ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof. In embodiments where L³ is a combination of any of the alternatives listed in its definition, L³ may include more than one occurrence of any listed alternative.

In certain embodiments, L³ is substituted or unsubstituted aliphatic, substituted or unsubstituted heteroaliphatic, substituted or unsubstituted heterocyclylene, substituted or unsubstituted heteroarylene, —O—, —N(R^{A})—, —C(═O)—, or —C(═O)NR^{A}—, or a combination thereof.

In certain embodiments, L³ is substituted or unsubstituted alkylene, substituted or unsubstituted heteroalkylene, substituted or unsubstituted heterocyclylene, substituted or unsubstituted heteroarylene, —O—, —N(R^{A})—, —C(═O)—, or —C(═O)N(R^{A})₂, or a combination thereof.

In certain embodiments, L³ is substituted or unsubstituted alkylene, substituted or unsubstituted heterocyclylene, substituted or unsubstituted heteroarylene, —O—, —N(R^{A})—, or —C(═O)N(R^{A})₂, or a combination thereof. In certain embodiments, L³ is unsubstituted alkylene, unsubstituted heterocyclylene, substituted heteroarylene, —O—, —N(R^{A})—, or —C(═O)N(R^{A})₂, or a combination thereof. In certain embodiments, L³ is a combination of substituted or unsubstituted alkylene, substituted or unsubstituted heterocyclylene, substituted or unsubstituted heteroarylene, —O—, —N(R^{A})—, and —C(═O)N(R^{A})₂. In certain embodiments, L³ is a combination of unsubstituted alkylene, unsubstituted heterocyclylene, substituted heteroarylene, —O—, —N(R^{A})—, and —C(═O)N(R^{A})₂.

In certain embodiments, L³ is substituted or unsubstituted alkylene, substituted or unsubstituted heteroalkylene, substituted or unsubstituted heterocyclylene, substituted or unsubstituted heteroarylene, —O—, —N(R^{A})—, or —C(═O)N(R^{A})₂, or a combination thereof. In certain embodiments, L³ is unsubstituted heteroalkylene, unsubstituted alkylene, unsubstituted heterocyclylene, substituted heteroarylene, —O—, —N(R^{A})—, or —C(═O)N(R^{A})₂, or a combination thereof. In certain embodiments, L³ is a combination of substituted or unsubstituted alkylene, substituted or unsubstituted heteroalkylene, substituted or unsubstituted heterocyclylene, substituted or unsubstituted heteroarylene, —O—, —N(R^{A})—, and —C(═O)N(R^{A})₂. In certain embodiments, L³ is a combination of unsubstituted alkylene, unsubstituted heteroalkylene, unsubstituted heterocyclylene, substituted heteroarylene, —O—, —N(R^{A})—, and —C(═O)N(R^{A})₂.

In certain embodiments, L³ is:

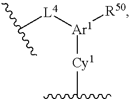

wherein:
L⁴ is —O—, substituted or unsubstituted heteroalkylene, or substituted or unsubstituted alkylene, or a combination thereof;
Cy¹ is substituted or unsubstituted heterocyclylene;
Ar¹ is substituted or unsubstituted heteroarylene; and
R⁵⁰ is substituted or unsubstituted heteroalkylene, substituted or unsubstituted alkylene, —N(R^{A})—, or —C(═O)N(R^{A})₂, or a combination thereof.

In certain embodiments, Ar¹ is unsubstituted heteroarylene. In certain embodiments, Ar¹ is unsubstituted pyridine. In certain embodiments, Ar¹ is unsubstituted pyrimidine. In certain embodiments, Ar¹ is unsubstituted triazine.

In certain embodiments, Cy¹ is unsubstituted heterocyclylene. In certain embodiments, Ar¹ is unsubstituted piperidine. In certain embodiments, Ar¹ is unsubstituted piperazine.

In certain embodiments, L⁴ is a combination of —O— and substituted or unsubstituted alkylene. In certain embodiments, L⁴ is a combination of —O— and unsubstituted alkylene. In certain embodiments, L⁴ is substituted or unsubstituted heteroalkylene. In certain embodiments, L⁴ is substituted or unsubstituted C₄-C₁₀ heteroalkylene. In certain embodiments, L⁴ is unsubstituted C₄-C₇ heteroalkylene. In certain embodiments, L⁴ is unsubstituted C₄-C₇ heteroalkylene, wherein each heteroatom of the heteroalkylene is independently oxygen or nitrogen. In certain embodiments, L⁴ is unsubstituted C₄-C₇ heteroalkylene, wherein each heteroatom of the heteroalkylene is oxygen. In certain embodiments, L⁴ is unsubstituted C₄-C₆ heteroalkylene. In certain embodiments, L⁴ is unsubstituted C₄-C₆ heteroalkylene, wherein each heteroatom of the heteroalkylene is independently oxygen or nitrogen. In certain embodiments, L⁴ is unsubstituted C₄-C₆ heteroalkylene, wherein each heteroatom of the heteroalkylene is oxygen. In certain embodiments, L⁴ is

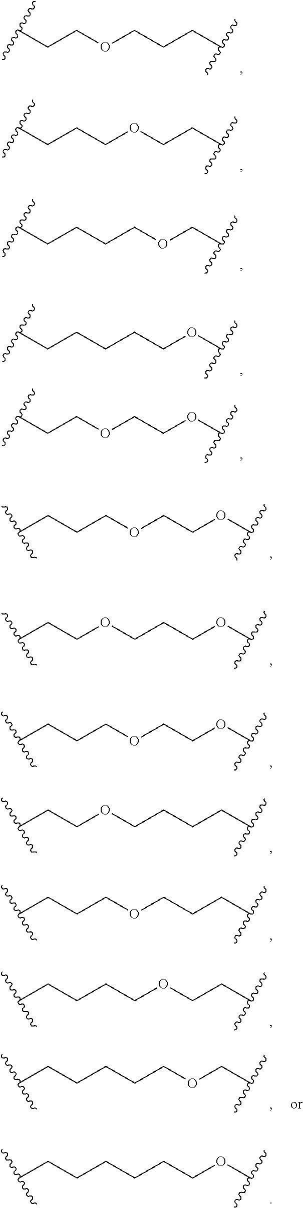

In certain embodiments, L⁴ is

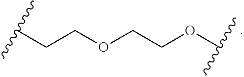 [SMILES: *CCOCCO*]

In certain embodiments, R⁵⁰ is substituted or unsubstituted heteroalkylene. In certain embodiments, R⁵⁰ is substituted or unsubstituted alkylene. In certain embodiments, R⁵⁰ is a combination of —N(R^{A})—, unsubstituted alkylene, and —C(═O)N(R^{A})₂. In certain embodiments, R⁵⁰ is

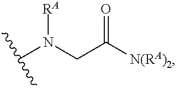

wherein R^{A} is independently hydrogen or alkyl. In certain embodiments, R⁵⁰ is

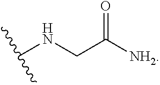 [SMILES: *NCC(N)=O]

In certain embodiments, L³ is:

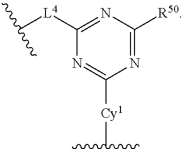

In certain embodiments, L³ is:

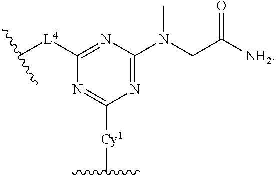

In certain embodiments, L³ is:

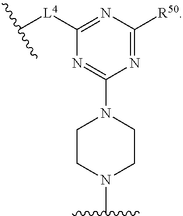

In certain embodiments, L³ is:

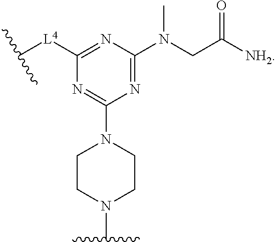

In certain embodiments, L³ is:

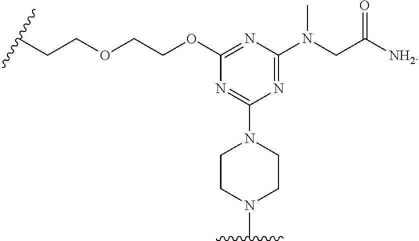 [SMILES: *CCOCCOc1nc(N(C)CC(N)=O)nc(N2CCN(*)CC2)n1]

In certain embodiments, L³ is substituted or unsubstituted alkylene. In certain embodiments, L³ is substituted or unsubstituted C₁₋₂₀ alkylene. In certain embodiments, L³ is substituted or unsubstituted C₁₋₁₅ alkylene. In certain embodiments, L³ is substituted or unsubstituted C₁₋₁₀ alkylene. In certain embodiments, L³ is substituted or unsubstituted C₁₋₆ alkylene. In certain embodiments, L³ is substituted or unsubstituted C₆ alkylene.

In certain embodiments, L³ is unsubstituted alkylene. In certain embodiments, L³ is unsubstituted C₁₋₂₀ alkylene. In certain embodiments, L³ is unsubstituted C₁₋₁₅ alkylene. In certain embodiments, L³ is unsubstituted C₁₋₁₀ alkylene. In certain embodiments, L³ is unsubstituted C₃₋₈ alkylene. In certain embodiments, L³ is unsubstituted C₁₋₆ alkylene. In certain embodiments, L³ is unsubstituted C₄₋₇ alkylene. In certain embodiments, L³ is unsubstituted C₄₋₆ alkylene. In certain embodiments, L³ is unsubstituted C₅₋₆ alkylene.

In certain embodiments, L³ is:

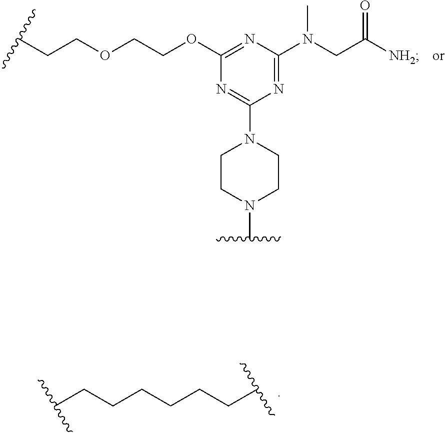 [SMILES: *CCOCCOc1nc(N(C)CC(N)=O)nc(N2CCN(*)CC2)n1 ; *CCCCCC*]

### Group X¹

As described herein, X¹ is a bond or a peptide. In certain embodiments, X¹ is a bond. In certain embodiments, X¹ is a peptide.

### Group Q

As described herein, Q is a targeting agent. In certain embodiments, Q is a protein. In certain embodiments, Q is a polypeptide. In certain embodiments, Q is a cell-targeting agent, e.g., muscle-targeting protein, e.g., for delivering an oligonucleotide to a muscle cell. In some embodiments, such cell-targeting proteins are capable of binding to a specific cell, e.g., via specifically binding to an antigen on said cell, and delivering an associated oligonucleotide to the cell. In some embodiments, the oligonucleotide is internalized into said cell upon binding of the cell-targeting agent to an antigen on the cell, e.g., via endocytosis.

a. Muscle-Targeting Agents

In certain embodiments, Q is a muscle targeting agent, e.g., for delivering a molecular payload to a muscle cell. In some embodiments, such muscle-targeting agents are capable of binding to a muscle cell, e.g., via specifically binding to an antigen on the muscle cell, and delivering an associated molecular payload to the muscle cell. In some embodiments, the molecular payload is bound (e.g., covalently bound) to the muscle targeting agent and is internalized into the muscle cell upon binding of the muscle targeting agent to an antigen on the muscle cell, e.g., via endocytosis. Exemplary muscle-targeting agents are described in further detail herein, however, it should be appreciated that the exemplary muscle-targeting agents provided herein are not meant to be limiting.

In certain embodiments, the muscle-targeting agents specifically bind to an antigen on muscle, such as skeletal muscle, smooth muscle, or cardiac muscle. In some embodiments, any of the muscle-targeting agents provided herein bind to (e.g., specifically bind to) an antigen on a skeletal muscle cell, a smooth muscle cell, and/or a cardiac muscle cell.

By interacting with muscle-specific cell surface recognition elements (e.g., cell membrane proteins), both tissue localization and selective uptake into muscle cells can be achieved. In some embodiments, molecules that are substrates for muscle uptake transporters are useful for delivering a molecular payload (e.g., oligonucleotide) into muscle tissue. Binding to muscle surface recognition elements followed by endocytosis can allow even large molecules such as antibodies to enter muscle cells. As another example oligonucleotides conjugated to transferrin or anti-transferrin receptor antibodies can be taken up by muscle cells via binding to transferrin receptor, which may then be endocytosed, e.g., via clathrin-mediated endocytosis.

The use of muscle-targeting agents may be useful for concentrating a molecular payload (e.g., oligonucleotide) in muscle while reducing toxicity associated with effects in other tissues. In some embodiments, the muscle-targeting agent concentrates a bound molecular payload in muscle cells as compared to another cell type within a subject. In some embodiments, the muscle-targeting agent concentrates a bound molecular payload in muscle cells (e.g., skeletal, smooth, or cardiac muscle cells) in an amount that is at least 1, 2, 3, 4, 5, 6, 7, 8, 9, 10, 15, 20, 30, 40, 50, 60, 70, 80, 90, or 100 times greater than an amount in non-muscle cells (e.g., liver, neuronal, blood, or fat cells). In some embodiments, a toxicity of the molecular payload in a subject is reduced by at least 1%, 2%, 3%, 4%, 5%, 10%, 15%, 20%, 25%, 30%, 35%, 40%, 45%, 50%, 55%, 60%, 65%, 70%, 75%, 80%, 90%, or 95% when it is delivered to the subject when bound to the muscle-targeting agent.

In some embodiments, to achieve muscle selectivity, a muscle recognition element (e.g., a muscle cell antigen) may be required. As one example, a muscle-targeting agent may be a small molecule that is a substrate for a muscle-specific uptake transporter. As another example, a muscle-targeting agent may be an antibody that enters a muscle cell via transporter-mediated endocytosis. As another example, a muscle targeting agent may be a ligand that binds to cell surface receptor on a muscle cell. It should be appreciated that while transporter-based approaches provide a direct path for cellular entry, receptor-based targeting may involve stimulated endocytosis to reach the desired site of action.

Muscle cells encompassed by the present disclosure include, but are not limited to, skeletal muscle cells, smooth muscle cells, cardiac muscle cells, myoblasts and myocytes.

b. Muscle-Targeting Antibodies

In some embodiments, the muscle-targeting agent is an antibody. Generally, the high specificity of antibodies for their target antigen provides the potential for selectively targeting muscle cells (e.g., skeletal, smooth, and/or cardiac muscle cells). This specificity may also limit off-target toxicity. Examples of antibodies that are capable of targeting a surface antigen of muscle cells have been reported and are within the scope of the disclosure. For example, antibodies that target the surface of muscle cells are described in Arahata K., et al. “Immunostaining of skeletal and cardiac muscle surface membrane with antibody against Duchenne muscular dystrophy peptide” Nature 1988; 333: 861-3; Song K. S., et al. “Expression of caveolin-3 in skeletal, cardiac, and smooth muscle cells. Caveolin-3 is a component of the sarcolemma and co-fractionates with dystrophin and dystrophin-associated glycoproteins” J Biol Chem 1996; 271: 15160-5; and Weisbart R. H. et al., “Cell type specific targeted intracellular delivery into muscle of a monoclonal antibody that binds myosin IIb” Mol Immunol. 2003 March, 39(13):78309; the entire contents of each of which are incorporated herein by reference.

c. Anti-Transferrin Receptor Antibodies

Some aspects of the disclosure provide transferrin receptor binding proteins, which are capable of binding to transferrin receptor. Accordingly, aspects of the disclosure provide binding proteins (e.g., antibodies) that bind to transferrin receptor. In some embodiments, binding proteins that bind to transferrin receptor are internalized, along with any bound molecular payload (e.g., oligonucleotide), into a muscle cell. Antibodies that bind, e.g. specifically bind, to a transferrin receptor may be internalized into the cell, e.g. through receptor-mediated endocytosis, upon binding to a transferrin receptor.

Any appropriate anti-transferrin receptor antibodies may be used in the complexes disclosed herein. Examples of anti-transferrin receptor antibodies, including associated references and binding epitopes, are listed in Table 1. In some embodiments, the anti-transferrin receptor antibody comprises the complementarity determining regions (CDR-H1, CDR-H2, CDR-H3, CDR-L1, CDR-L2, and CDR-L3) of any of the anti-transferrin receptor antibodies provided herein, e.g., anti-transferrin receptor antibodies listed in Table 1.

TABLE 1
List of anti-transferrin receptor antibody clones, including
associated references and binding epitope information.

| Antibody | | |
|---|---|---|
| Clone Name | Reference(s) | Epitope/Notes |
| OKT9 | U.S. Pat. No. 4,364,934, filed Dec. 4, 1979, | Apical domain of TfR |
| | entitled “MONOCLONAL ANTIBODY | (residues 305-366 of |
| | TO A HUMAN EARLY THYMOCYTE | human TfR sequence |
| | ANTIGEN AND METHODS FOR | XM_052730.3, |
| | PREPARING SAME” | available in GenBank) |
| | Schneider C. et al. “Structural features of | |
| | the cell surface receptor for transferrin that | |
| | is recognized by the monoclonal antibody | |
| | OKT9.” J Biol Chem. 1982, 257: 14, 8516- | |
| | 8522. | |
| | WO 2015/098989, filed | |
| (From JCR) | WO 2015/098989, filed | Apical domain |
| Clone M11 | Dec. 24, 2014, “Novel anti-Transferrin | (residues 230-244 and |
| Clone M23 | receptor antibody that passes through | 326-347 of TfR) and |
| Clone M27 | blood-brain barrier” | protease-like domain |
| Clone B84 | U.S. Pat. No. 9,994,641, filed | (residues 461-473) |
| | Dec. 24, 2014, “Novel anti-Transferrin | |
| | receptor antibody that passes through | |
| | blood-brain barrier” | |
| (From | WO 2016/081643, filed May 26, 2016, | Apical domain and |
| Genentech) | entitled “ANTI-TRANSFERRIN | non-apical regions |
| 7A4, 8A2, | RECEPTOR ANTIBODIES AND | |
| 15D2, 10D11, | METHODS OF USE” | |
| 7B10, 15G11, | U.S. Pat. No. 9,708,406, filed | |
| 16G5, 13C3, | May 20, 2014, “Anti-transferrin receptor | |
| 16G4, 16F6, | antibodies and methods of use” | |
| 7G7, 4C2, | | |
| 1B12, and | | |
| 13D4 | | |
| (From | Lee et al. “Targeting Rat Anti- | |
| Armagen) | Mouse Transferrin Receptor Monoclonal | |
| 8D3 | Antibodies through Blood-Brain Barrier in | |
| | Mouse” 2000, J Pharmacol. Exp. Ther., | |
| | 292: 1048-1052. | |
| | US Patent App. 2010/077498, filed | |
| | Sep. 11, 2008, entitled “COMPOSITIONS | |
| | AND METHODS FOR BLOOD-BRAIN | |
| | BARRIER DELIVERY IN THE MOUSE” | |
| OX26 | Haobam, B. et al. 2014. Rab17- | |
| | mediated recycling endosomes contribute | |
| | to autophagosome formation in response to | |
| | Group A Streptococcus invasion. Cellular | |
| | microbiology. 16: 1806-21. | |
| DF1513 | Ortiz-Zapater E et al. Trafficking | |
| | of the human transferrin receptor in plant | |
| | cells: effects of tyrphostin A23 and | |
| | brefeldin A. Plant J 48: 757-70 (2006). | |
| 1A1B2, | Commercially available anti- | Novus Biologicals |
| 66IG10, | transferrin receptor antibodies. | 8100 Southpark Way, |
| MEM-189, | | A-8 Littleton CO |
| JF0956, 29806, | | 80120 |
| 1A1B2, | | |
| TFRC/1818, | | |
| 1E6, 66Ig10, | | |
| TFRC/1059, | | |
| Q1/71, 23D10, | | |
| 13E4, | | |
| TFRC/1149, | | |
| ER-MP21, | | |
| YTA74.4, | | |
| BU54, 2B6, | | |
| RI7 217 | | |
| (From | US Patent App. 2011/0311544A1, | Does not compete |
| INSERM) | filed Jun. 26, 2005, entitled “ANTI-CD71 | with OKT9 |
| BA120g | MONOCLONAL ANTIBODIES AND | |
| | USES THEREOF FOR TREATING | |
| | MALIGNANT TUMOR CELLS” | |
| LUCA31 | U.S. Pat. No. 7,572,895, filed | “LUCA31 epitope” |
| | Jun. 7, 2004, entitled “TRANSFERRIN | |
| | RECEPTOR ANTIBODIES” | |
| (Salk Institute) | Trowbridge, I. S. et al. “Anti-transferrin | |
| B3/25 | receptor monoclonal antibody and | |
| T58/30 | toxin-antibody conjugates affect | |
| | growth of human tumour cells.” | |
| | Nature, 1981, volume 294, pages | |
| | 171-173 | |
| R17 217.1.3, | Commercially available anti- | BioXcell |
| 5E9C11, | transferrin receptor antibodies. | 10 Technology Dr., |
| OKT9 | | Suite 2B |
| (BE0023 | | West Lebanon, NH |
| clone) | | 03784-1671 USA |
| BK19.9, | Gatter, K. C. et al. “Transferrin | |
| B3/25, T56/14 | receptors in human tissues: their | |
| and T58/1 | distribution and possible clinical | |
| | relevance.” J Clin Pathol. 1983 | |
| | May; 36(5): 539-45. | |

In some embodiments, transferrin receptor antibodies of the present disclosure include one or more of the CDR-H (e.g., CDR-H1, CDR-H2, and CDR-H3) amino acid sequences from any one of the anti-transferrin receptor antibodies selected from Table 1. In some embodiments, transferrin receptor antibodies include the CDR-H1, CDR-H2, and CDR-H3 as provided for any one of the anti-transferrin receptor antibodies selected from Table 1. In some embodiments, anti-transferrin receptor antibodies include the CDR-L1, CDR-L2, and CDR-L3 as provided for any one of the anti-transferrin receptor antibodies selected from Table 1. In some embodiments, anti-transferrin antibodies include the CDR-H1, CDR-H2, CDR-H3, CDR-L1, CDR-L2, and CDR-L3 as provided for any one of the anti-transferrin receptor antibodies selected from Table 1. The disclosure also includes any nucleic acid sequence that encodes a molecule comprising a CDR-H1, CDR-H2, CDR-H3, CDR-L1, CDR-L2, or CDR-L3 as provided for any one of the anti-transferrin receptor antibodies selected from Table 1. In some embodiments, antibody heavy and light chain CDR3 domains may play a particularly important role in the binding specificity/affinity of an antibody for an antigen. Accordingly, anti-transferrin receptor antibodies of the disclosure may include at least the heavy and/or light chain CDR3s of any one of the anti-transferrin receptor antibodies selected from Table 1.

In some embodiments, the muscle-targeting agent is a transferrin receptor antibody (e.g., the antibody and variants thereof as described in International Application Publication WO 2016/081643, incorporated herein by reference). In certain embodiments, the muscle-targeting agent is any transferrin receptor antibody disclosed in International Application Publications WO 2020/028861, WO 2020/028864, WO 2020/028844, WO 2020/028841, WO 2020/028831, WO 2020/028840, WO 2020/028857, WO 2020/028836, WO 2020/028832, and WO 2020/028842, each of which is incorporated herein by reference.

d. Other Muscle-Targeting Antibodies

In some embodiments, the muscle-targeting antibody is an antibody that specifically binds hemojuvelin, caveolin-3, Duchenne muscular dystrophy peptide, myosin IIB or CD63. In some embodiments, the muscle-targeting antibody is an antibody that specifically binds a myogenic precursor protein. Exemplary myogenic precursor proteins include, without limitation, ABCG2, M-Cadherin/Cadherin-15, Caveolin-1, CD34, FoxK1, Integrin alpha 7, Integrin alpha 7 beta 1, MYF-5, MyoD, Myogenin, NCAM-1/CD56, Pax3, Pax7, and Pax9. In some embodiments, the muscle-targeting antibody is an antibody that specifically binds a skeletal muscle protein. Exemplary skeletal muscle proteins include, without limitation, alpha-Sarcoglycan, beta-Sarcoglycan, Calpain Inhibitors, Creatine Kinase MM/CKMM, eIF5A, Enolase 2/Neuron-specific Enolase, epsilon-Sarcoglycan, FABP3/H-FABP, GDF-8/Myostatin, GDF-11/GDF-8, Integrin alpha 7, Integrin alpha 7 beta 1, Integrin beta 1/CD29, MCAM/CD146, MyoD, Myogenin, Myosin Light Chain Kinase Inhibitors, NCAM-1/CD56, and Troponin I. In some embodiments, the muscle-targeting antibody is an antibody that specifically binds a smooth muscle protein. Exemplary smooth muscle proteins include, without limitation, alpha-Smooth Muscle Actin, VE-Cadherin, Caldesmon/CALD1, Calponin 1, Desmin, Histamine H2 R, Motilin R/GPR38, Transgelin/TAGLN, and Vimentin. However, it should be appreciated that antibodies to additional targets are within the scope of this disclosure and the exemplary lists of targets provided herein are not meant to be limiting.

e. Muscle-Targeting Peptides

Some aspects of the disclosure provide muscle-targeting peptides as muscle-targeting agents. Short peptide sequences (e.g., peptide sequences of 5-20 amino acids in length) that bind to specific cell types have been described. For example, cell-targeting peptides have been described in Vines e., et al., A. “Cell-penetrating and cell-targeting peptides in drug delivery” Biochim Biophys Acta 2008, 1786: 126-38; Jarver P., et al., “In vivo biodistribution and efficacy of peptide mediated delivery” Trends Pharmacol Sci 2010; 31: 528-35; Samoylova T. I., et al., “Elucidation of muscle-binding peptides by phage display screening” Muscle Nerve 1999; 22: 460-6; U.S. Pat. No. 6,329,501, issued on Dec. 11, 2001, entitled “METHODS AND COMPOSITIONS FOR TARGETING COMPOUNDS TO MUSCLE”; and Samoylov A. M., et al., “Recognition of cell-specific binding of phage display derived peptides using an acoustic wave sensor.” Biomol Eng 2002; 18: 269-72; the entire contents of each of which are incorporated herein by reference. By designing peptides to interact with specific cell surface antigens (e.g., receptors), selectivity for a desired tissue, e.g., muscle, can be achieved. Skeletal muscle-targeting has been investigated and a range of molecular payloads are able to be delivered. These approaches may have high selectivity for muscle tissue without many of the practical disadvantages of a large antibody or viral particle. Accordingly, in some embodiments, the muscle-targeting agent is a muscle-targeting peptide that is from 4 to 50 amino acids in length. In some embodiments, the muscle-targeting peptide is 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. Muscle-targeting peptides can be generated using any of several methods, such as phage display.

In some embodiments, a muscle-targeting peptide may bind to an internalizing cell surface receptor that is overexpressed or relatively highly expressed in muscle cells, e.g., a transferrin receptor, compared with certain other cells. In some embodiments, a muscle-targeting peptide may target, e.g., bind to, a transferrin receptor. In some embodiments, a peptide that targets a transferrin receptor may comprise a segment of a naturally occurring ligand, e.g., transferrin. In some embodiments, a peptide that targets a transferrin receptor is as described in U.S. Pat. No. 6,743,893, filed Nov. 30, 2000, “RECEPTOR-MEDIATED UPTAKE OF PEPTIDES THAT BIND THE HUMAN TRANSFERRIN RECEPTOR”. In some embodiments, a peptide that targets a transferrin receptor is as described in Kawamoto, M. et al, “A novel transferrin receptor-targeted hybrid peptide disintegrates cancer cell membrane to induce rapid killing of cancer cells.” BMC Cancer. 2011 Aug. 18; 11:359. In some embodiments, a peptide that targets a transferrin receptor is as described in U.S. Pat. No. 8,399,653, filed May 20, 2011, “TRANSFERRIN/TRANSFERRIN RECEPTOR-MEDIATED SIRNA DELIVERY”.

As discussed above, examples of muscle targeting peptides have been reported. For example, muscle-specific peptides were identified using phage display library presenting surface heptapeptides. As one example a peptide having the amino acid sequence ASSLNIA (SEQ ID NO: 1) bound to C2C12 murine myotubes in vitro, and bound to mouse muscle tissue in vivo. Accordingly, in some embodiments, the muscle-targeting agent comprises the amino acid sequence ASSLNIA (SEQ ID NO: 1). This peptide displayed improved specificity for binding to heart and skeletal muscle tissue after intravenous injection in mice with reduced binding to liver, kidney, and brain. Additional muscle-specific peptides have been identified using phage display. For example, a 12 amino acid peptide was identified by phage display library for muscle targeting in the context of treatment for DMD. See, Yoshida D., et al., “Targeting of salicylate to skin and muscle following topical injections in rats.” Int J Pharm 2002; 231: 177-84; the entire contents of which are hereby incorporated by reference. Here, a 12 amino acid peptide having the sequence SKTFNTHPQSTP (SEQ ID NO: 2) was identified and this muscle-targeting peptide showed improved binding to C2C12 cells relative to the ASSLNIA (SEQ ID NO: 1) peptide.

An additional method for identifying peptides selective for muscle (e.g., skeletal muscle) over other cell types includes in vitro selection, which has been described in Ghosh D., et al., “Selection of muscle-binding peptides from context-specific peptide-presenting phage libraries for adenoviral vector targeting” J Virol 2005; 79: 13667-72; the entire contents of which are incorporated herein by reference. By pre-incubating a random 12-mer peptide phage display library with a mixture of non-muscle cell types, non-specific cell binders were selected out. Following rounds of selection, the 12 amino acid peptide TARGEHKEEELI (SEQ ID NO: 3) appeared most frequently. Accordingly, in some embodiments, the muscle-targeting agent comprises the amino acid sequence TARGEHKEEELI (SEQ ID NO: 3).

A muscle-targeting agent may be an amino acid-containing molecule or peptide. A muscle-targeting peptide may correspond to a sequence of a protein that preferentially binds to a protein receptor found in muscle cells. In some embodiments, a muscle-targeting peptide contains a high propensity of hydrophobic amino acids, e.g. valine, such that the peptide preferentially targets muscle cells. In some embodiments, a muscle-targeting peptide has not been previously characterized or disclosed. These peptides may be conceived of, produced, synthesized, and/or derivatized using any of several methodologies, e.g. phage displayed peptide libraries, one-bead one-compound peptide libraries, or positional scanning synthetic peptide combinatorial libraries. Exemplary methodologies have been characterized in the art and are incorporated by reference (Gray, B. P. and Brown, K. C. “Combinatorial Peptide Libraries: Mining for Cell-Binding Peptides” Chem Rev. 2014, 114:2, 1020-1081; Samoylova, T. I. and Smith, B. F. “Elucidation of muscle-binding peptides by phage display screening.” Muscle Nerve, 1999, 22:4. 460-6). In some embodiments, a muscle-targeting peptide has been previously disclosed (see, e.g. Writer M. J. et al. “Targeted gene delivery to human airway epithelial cells with synthetic vectors incorporating novel targeting peptides selected by phage display.” J. Drug Targeting. 2004; 12:185; Cai, D. “BDNF-mediated enhancement of inflammation and injury in the aging heart.” Physiol Genomics. 2006, 24:3, 191-7; Zhang, L. “Molecular profiling of heart endothelial cells.” Circulation, 2005, 112:11, 1601-11; McGuire, M. J. et al. “In vitro selection of a peptide with high selectivity for cardiomyocytes in vivo.” J Mol Biol. 2004, 342:1, 171-82). Exemplary muscle-targeting peptides comprise an amino acid sequence of the following group: CQAQGQLVC (SEQ ID NO: 4), CSERSMNFC (SEQ ID NO: 5), CPKTRRVPC (SEQ ID NO: 6), WLSEAGPVVTVRALRGTGSW (SEQ ID NO: 7), ASSLNIA (SEQ ID NO: 1), CMQHSMRVC (SEQ ID NO: 8), and DDTRHWG (SEQ ID NO: 9). In some embodiments, a muscle-targeting peptide may comprise about 2-25 amino acids, about 2-20 amino acids, about 2-15 amino acids, about 2-10 amino acids, or about 2-5 amino acids. Muscle-targeting peptides may comprise naturally-occurring amino acids, e.g. cysteine, alanine, or non-naturally-occurring or modified amino acids. Non-naturally occurring amino acids include (3-amino acids, homo-amino acids, proline derivatives, 3-substituted alanine derivatives, linear core amino acids, N-methyl amino acids, and others known in the art. In some embodiments, a muscle-targeting peptide may be linear; in other embodiments, a muscle-targeting peptide may be cyclic, e.g. bicyclic (see, e.g. Silvana, M. G. et al. Mol. Therapy, 2018, 26:1, 132-147). A muscle-targeting agent may be an aptamer, e.g. a peptide aptamer, which preferentially targets muscle cells relative to other cell types.

f. Muscle-Targeting Receptor Ligands

A muscle-targeting agent may be a ligand, e.g. a ligand that binds to a receptor protein. A muscle-targeting ligand may be a protein, e.g. transferrin, which binds to an internalizing cell surface receptor expressed by a muscle cell. Accordingly, in some embodiments, the muscle-targeting agent is transferrin, or a derivative thereof that binds to a transferrin receptor. A muscle-targeting ligand may alternatively be a small molecule, e.g. a lipophilic small molecule that preferentially targets muscle cells relative to other cell types. Exemplary lipophilic small molecules that may target muscle cells include compounds comprising cholesterol, cholesteryl, stearic acid, palmitic acid, oleic acid, oleyl, linolene, linoleic acid, myristic acid, sterols, dihydrotestosterone, testosterone derivatives, glycerine, alkyl chains, trityl groups, and alkoxy acids.

g. Other Muscle-Targeting Agents

One strategy for targeting a muscle cell (e.g., a skeletal muscle cell) is to use a substrate of a muscle transporter protein, such as a transporter protein expressed on the sarcolemma. In some embodiments, the muscle-targeting agent is a substrate of an influx transporter that is specific to muscle tissue. In some embodiments, the influx transporter is specific to skeletal muscle tissue. Two main classes of transporters are expressed on the skeletal muscle sarcolemma, (1) the adenosine triphosphate (ATP) binding cassette (ABC) superfamily, which facilitate efflux from skeletal muscle tissue and (2) the solute carrier (SLC) superfamily, which can facilitate the influx of substrates into skeletal muscle. In some embodiments, the muscle-targeting agent is a substrate that binds to an ABC superfamily or an SLC superfamily of transporters. In some embodiments, the substrate that binds to the ABC or SLC superfamily of transporters is a naturally-occurring substrate. In some embodiments, the substrate that binds to the ABC or SLC superfamily of transporters is a non-naturally occurring substrate, for example, a synthetic derivative thereof that binds to the ABC or SLC superfamily of transporters.

In some embodiments, the muscle-targeting agent is a substrate of an SLC superfamily of transporters. SLC transporters are either equilibrative or use proton or sodium ion gradients created across the membrane to drive transport of substrates. Exemplary SLC transporters that have high skeletal muscle expression include, without limitation, the SATT transporter (ASCT1; SLC1A4), GLUT4 transporter (SLC2A4), GLUT7 transporter (GLUT7; SLC2A7), ATRC2 transporter (CAT-2; SLC7A2), LAT3 transporter (KIAA0245; SLC7A6), PHT1 transporter (PTR4; SLC15A4), OATP-J transporter (OATP5A1; SLC21A15), OCT3 transporter (EMT; SLC22A3), OCTN2 transporter (FLJ46769; SLC22A5), ENT transporters (ENT1; SLC29A1 and ENT2; SLC29A2), PAT2 transporter (SLC36A2), and SAT2 transporter (KIAA1382; SLC38A2). These transporters can facilitate the influx of substrates into skeletal muscle, providing opportunities for muscle targeting.

In some embodiments, the muscle-targeting agent is a substrate of an equilibrative nucleoside transporter 2 (ENT2) transporter. Relative to other transporters, ENT2 has one of the highest mRNA expressions in skeletal muscle. While human ENT2 (hENT2) is expressed in most body organs such as brain, heart, placenta, thymus, pancreas, prostate, and kidney, it is especially abundant in skeletal muscle. Human ENT2 facilitates the uptake of its substrates depending on their concentration gradient. ENT2 plays a role in maintaining nucleoside homeostasis by transporting a wide range of purine and pyrimidine nucleobases. The hENT2 transporter has a low affinity for all nucleosides (adenosine, guanosine, uridine, thymidine, and cytidine) except for inosine. Accordingly, in some embodiments, the muscle-targeting agent is an ENT2 substrate. Exemplary ENT2 substrates include, without limitation, inosine, 2′,3′-dideoxyinosine, and calofarabine. In some embodiments, any of the muscle-targeting agents provided herein are associated with a molecular payload (e.g., oligonucleotide payload). In some embodiments, the muscle-targeting agent is covalently linked to the molecular payload. In some embodiments, the muscle-targeting agent is non-covalently linked to the molecular payload.

In some embodiments, the muscle-targeting agent is a substrate of an organic cation/carnitine transporter (OCTN2), which is a sodium ion-dependent, high affinity carnitine transporter. In some embodiments, the muscle-targeting agent is carnitine, mildronate, acetylcarnitine, or any derivative thereof that binds to OCTN2. In some embodiments, the carnitine, mildronate, acetylcarnitine, or derivative thereof is covalently linked to the molecular payload (e.g., oligonucleotide payload).

A muscle-targeting agent may be a protein that is protein that exists in at least one soluble form that targets muscle cells. In some embodiments, a muscle-targeting protein may be hemojuvelin (also known as repulsive guidance molecule C or hemochromatosis type 2 protein), a protein involved in iron overload and homeostasis. In some embodiments, hemojuvelin may be full length or a fragment, or a mutant with at least 75%, at least 80%, at least 85%, at least 90%, at least 95%, at least 98% or at least 99% sequence identity to a functional hemojuvelin protein. In some embodiments, a hemojuvelin mutant may be a soluble fragment, may lack a N-terminal signaling, and/or lack a C-terminal anchoring domain. In some embodiments, hemojuvelin may be annotated under GenBank RefSeq Accession Numbers NM_001316767.1, NM_145277.4, NM_202004.3, NM_213652.3, or NM_213653.3. It should be appreciated that a hemojuvelin may be of human, non-human primate, or rodent origin.

In certain embodiments, Q is any of the targeting agents described above (e.g., muscle-targeting agent, antibody, protein, or ligand). In some embodiments, the antibody is a full-length IgG, a Fab fragment, a Fab′ fragment, a F(ab′)2 fragment, a scFv, or a Fv fragment. In some embodiments, the antibody is an anti-transferrin receptor (anti-TfR) antibody.

### Group R

As described herein, R is a molecular payload, e.g., for modulating a biological outcome, e.g., the transcription of a DNA sequence, the expression of a protein, or the activity of a protein. For example, the molecular payload may comprise, or consist of, an oligonucleotide (e.g., antisense oligonucleotide), a peptide (e.g., a peptide that binds a nucleic acid or protein associated with disease in a muscle cell), a protein (e.g., a protein that binds a nucleic acid or protein associated with disease in a muscle cell), or a small molecule (e.g., a small molecule that modulates the function of a nucleic acid or protein associated with disease in a muscle cell). In some embodiments, such molecular payloads are capable of targeting to a muscle cell, e.g., via specifically binding to a nucleic acid or protein in the muscle cell following delivery to the muscle cell by an associated muscle-targeting agent. In some embodiments, the molecular payload is an oligonucleotide that comprises a strand having a region of complementarity to a gene provided in Table 3. Exemplary molecular payloads are described in further detail herein, however, it should be appreciated that the exemplary molecular payloads provided herein are not meant to be limiting.

a. Oligonucleotides

Any suitable oligonucleotide may be used as a molecular payload, as described herein. In some embodiments, the oligonucleotide may be designed to cause degradation of an mRNA (e.g., the oligonucleotide may be a gapmer, an siRNA, a ribozyme or an aptamer that causes degradation). In some embodiments, the oligonucleotide may be designed to block translation of an mRNA (e.g., the oligonucleotide may be a mixmer, an siRNA or an aptamer that blocks translation). In some embodiments, an oligonucleotide may be designed to caused degradation and block translation of an mRNA. In some embodiments, an oligonucleotide may be a guide nucleic acid (e.g., guide RNA) for directing activity of an enzyme (e.g., a gene editing enzyme). Other examples of oligonucleotides are provided herein. It should be appreciated that, in some embodiments, oligonucleotides in one format (e.g., antisense oligonucleotides) may be suitably adapted to another format (e.g., siRNA oligonucleotides) by incorporating functional sequences (e.g., antisense strand sequences) from one format to the other format.

In some embodiments, an oligonucleotide may comprise a region of complementarity to a target gene provided in Table 2. In certain embodiments, the molecular payload is any oligonucleotide disclosed in International Application Publications WO 2020/028861, WO 2020/028864, WO 2020/028844, WO 2020/028841, WO 2020/028831, WO 2020/028840, WO 2020/028857, WO 2020/028836, WO 2020/028832, and WO 2020/028842, each of which is incorporated herein by reference.

b. Oligonucleotide Size/Sequence

Oligonucleotides may be of a variety of different lengths, e.g., depending on the format. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the oligonucleotide is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 21 to 23 nucleotides in lengths, etc.

In some embodiments, a complementary nucleic acid sequence of an oligonucleotide for purposes of the present disclosure is specifically hybridizable or specific for the target nucleic acid when binding of the sequence to the target molecule (e.g., mRNA) interferes with the normal function of the target (e.g., mRNA) to cause a loss of activity (e.g., inhibiting translation) or expression (e.g., degrading a target mRNA) and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency. Thus, in some embodiments, an oligonucleotide may be at least 80%, at least 85%, at least 90%, at least 91%, at least 92%, at least 93%, at least 94%, at least 95%, at least 96%, at least 97%, at least 98%, at least 99% or 100% complementary to the consecutive nucleotides of a target nucleic acid. In some embodiments a complementary nucleotide sequence need not be 100% complementary to that of its target to be specifically hybridizable or specific for a target nucleic acid.

In some embodiments, an oligonucleotide comprises a region of complementarity to a target nucleic acid that is in the range of 8 to 15, 8 to 30, 8 to 40, or 10 to 50, or 5 to 50, or 5 to 40 nucleotides in length. In some embodiments, a region of complementarity of an oligonucleotide to a target nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 8 consecutive nucleotides of a target nucleic acid. In some embodiments, an oligonucleotide may contain 1, 2 or 3 base mismatches compared to the portion of the consecutive nucleotides of target nucleic acid. In some embodiments the oligonucleotide may have up to 3 mismatches over 15 bases, or up to 2 mismatches over 10 bases.

In some embodiments, the oligonucleotide is complementary (e.g., at least 85% at least 90%, at least 95%, or 100%) to a target sequence of any one of the oligonucleotides provided herein. In some embodiments, such target sequence is 100% complementary to the oligonucleotide described herein.

In some embodiments, any one or more of the thymine bases (T's) in any one of the oligonucleotides provided herein may optionally be uracil bases (U's), and/or any one or more of the U's may optionally be T's.

c. Oligonucleotide Modifications

The oligonucleotides described herein may be modified, e.g., comprise a modified sugar moiety, a modified internucleoside linkage, a modified nucleotide and/or combinations thereof. In addition, in some embodiments, oligonucleotides may exhibit one or more of the following properties: do not mediate alternative splicing; are not immune stimulatory; are nuclease resistant; have improved cell uptake compared to unmodified oligonucleotides; are not toxic to cells or mammals; have improved endosomal exit internally in a cell; minimizes TLR stimulation; or avoid pattern recognition receptors. Any of the modified chemistries or formats of oligonucleotides described herein can be combined with each other. For example, one, two, three, four, five, or more different types of modifications can be included within the same oligonucleotide.

In some embodiments, certain nucleotide modifications may be used that make an oligonucleotide into which they are incorporated more resistant to nuclease digestion than the native oligodeoxynucleotide or oligoribonucleotide molecules; these modified oligonucleotides survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. Accordingly, oligonucleotides of the disclosure can be stabilized against nucleolytic degradation such as by the incorporation of a modification, e.g., a nucleotide modification.

In some embodiments, an oligonucleotide may be of up to 50 or up to 100 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are modified nucleotides. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are modified nucleotides. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are modified nucleotides. Optionally, the oligonucleotides may have every nucleotide except 1, 2, 3, 4, 5, 6, 7, 8, 9, or 10 nucleotides modified. Oligonucleotide modifications are described further herein.

In some embodiments, an oligonucleotide includes a 2′-modified nucleotide, e.g., a 2′-deoxy, 2′-fluoro, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA).

In some embodiments, an oligonucleotide can include at least one 2′-O-methyl-modified nucleotide, and in some embodiments, all of the nucleotides include a 2′-O-methyl modification. In some embodiments, an oligonucleotide comprises modified nucleotides in which the ribose ring comprises a bridge moiety connecting two atoms in the ring, e.g., connecting the 2′-O atom to the 4′-C atom. In some embodiments, the oligonucleotides are “locked,” e.g., comprise modified nucleotides in which the ribose ring is “locked” by a methylene bridge connecting the 2′-O atom and the 4′-C atom. Examples of LNAs are described in International Patent Application Publication WO/2008/043753, published on Apr. 17, 2008, and entitled “RNA Antagonist Compounds For The Modulation Of PCSK9”, the contents of which are incorporated herein by reference in its entirety.

Other modifications that may be used in the oligonucleotides disclosed herein include ethylene-bridged nucleic acids (ENAs). ENAs include, but are not limited to, 2′-O,4′-C-ethylene-bridged nucleic acids. Examples of ENAs are provided in International Patent Publication No. WO 2005/042777, published on May 12, 2005, and entitled “APP/ENA Antisense”; Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties.

In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. In some embodiments, the oligonucleotide comprises a modified nucleotide disclosed in one of the following United States Patent or Patent Application Publications: U.S. Pat. No. 7,399,845, issued on Jul. 15, 2008, and entitled “6-Modified Bicyclic Nucleic Acid Analogs”; U.S. Pat. No. 7,741,457, issued on Jun. 22, 2010, and entitled “6-Modified Bicyclic Nucleic Acid Analogs”; U.S. Pat. No. 8,022,193, issued on Sep. 20, 2011, and entitled “6-Modified Bicyclic Nucleic Acid Analogs”; U.S. Pat. No. 7,569,686, issued on Aug. 4, 2009, and entitled “Compounds And Methods For Synthesis Of Bicyclic Nucleic Acid Analogs”; U.S. Pat. No. 7,335,765, issued on Feb. 26, 2008, and entitled “Novel Nucleoside And Oligonucleotide Analogues”; U.S. Pat. No. 7,314,923, issued on Jan. 1, 2008, and entitled “Novel Nucleoside And Oligonucleotide Analogues”; U.S. Pat. No. 7,816,333, issued on Oct. 19, 2010, and entitled “Oligonucleotide Analogues And Methods Utilizing The Same” and US Publication Number 2011/0009471 now U.S. Pat. No. 8,957,201, issued on Feb. 17, 2015, and entitled “Oligonucleotide Analogues And Methods Utilizing The Same”, the entire contents of each of which are incorporated herein by reference for all purposes.

In some embodiments, the oligonucleotide comprises at least one nucleotide modified at the 2′ position of the sugar, preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA.

In some embodiments, the oligonucleotide may have at least one modified nucleotide that results in an increase in Tm of the oligonucleotide in a range of 1° C., 2° C., 3° C., 4° C., or 5° C. compared with an oligonucleotide that does not have the at least one modified nucleotide. The oligonucleotide may have a plurality of modified nucleotides that result in a total increase in Tm of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the modified nucleotide.

The oligonucleotide may comprise alternating nucleotides of different kinds. For example, an oligonucleotide may comprise alternating deoxyribonucleotides or ribonucleotides and 2′-fluoro-deoxyribonucleotides. An oligonucleotide may comprise alternating deoxyribonucleotides or ribonucleotides and 2′-O-methyl nucleotides. An oligonucleotide may comprise alternating 2′-fluoro nucleotides and 2′-O-methyl nucleotides. An oligonucleotide may comprise alternating bridged nucleotides and 2′-fluoro or 2′-O-methyl nucleotides.

d. Internucleotide Linkages/Backbones

In some embodiments, the oligonucleotide may contain a phosphorothioate or other modified internucleotide linkage. In some embodiments, the oligonucleotide comprises phosphorothioate internucleoside linkages. In some embodiments, the oligonucleotide comprises phosphorothioate internucleoside linkages between at least two nucleotides. In some embodiments, the oligonucleotide comprises phosphorothioate internucleoside linkages between all nucleotides. For example, in some embodiments, oligonucleotides comprise modified internucleotide linkages at the first, second, and/or third internucleoside linkage at the 5′ or 3′ end of the nucleotide sequence.

Phosphorous-containing linkages that may be used include, but are not limited to, phosphorothioates, chiral phosphorothioates, phosphorodithioates, phosphotriesters, aminoalkylphosphotriesters, methyl and other alkyl phosphonates comprising 3′-alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

In some embodiments, oligonucleotides may have heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497).

e. Stereospecific Oligonucleotides

In some embodiments, internucleotidic phosphorus atoms of oligonucleotides are chiral, and the properties of the oligonucleotides are adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43.) In some embodiments, phosphorothioate containing oligonucleotides are provided that comprise nucleoside units that are joined together by either substantially all Sp or substantially all Rp phosphorothioate intersugar linkages. In some embodiments, such phosphorothioate oligonucleotides having substantially chirally pure intersugar linkages are prepared by enzymatic or chemical synthesis, as described, for example, in U.S. Pat. No. 5,587,261, issued on Dec. 12, 1996, the contents of which are incorporated herein by reference in their entirety. In some embodiments, chirally controlled oligonucleotides provide selective cleavage patterns of a target nucleic acid. For example, in some embodiments, a chirally controlled oligonucleotide provides single site cleavage within a complementary sequence of a nucleic acid, as described, for example, in US Patent Application Publication 20170037399 A1, published on Feb. 2, 2017, entitled “CHIRAL DESIGN”, the contents of which are incorporated herein by reference in their entirety.

f. Morpholinos

In some embodiments, the oligonucleotide may be a morpholino-based compound. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

g. Peptide Nucleic Acids (PNAs)

In some embodiments, both a sugar and an internucleoside linkage (the backbone) of the nucleotide units of an oligonucleotide are replaced with novel groups. In some embodiments, the base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative publication that report the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

h. Gapmers

In some embodiments, the oligonucleotide is a gapmer. A gapmer oligonucleotide generally has the formula 5′-X-Y-Z-3′, with X and Z as flanking regions around a gap region Y. In some embodiments, the Y region is a contiguous stretch of nucleotides, e.g., a region of at least 6 DNA nucleotides, which are capable of recruiting an RNAse, such as RNAse H. In some embodiments, the gapmer binds to the target nucleic acid, at which point an RNAse is recruited and can then cleave the target nucleic acid. In some embodiments, the Y region is flanked both 5′ and 3′ by regions X and Z comprising high-affinity modified nucleotides, e.g., one to six modified nucleotides. Examples of modified nucleotides include, but are not limited to, 2′ MOE or 2′OMe or Locked Nucleic Acid bases (LNA). The flanking sequences X and Z may be of one to twenty nucleotides, one to eight nucleotides or one to five nucleotides in length, in some embodiments. The flanking sequences X and Z may be of similar length or of dissimilar lengths. The gap-segment Y may be a nucleotide sequence of five to twenty nucleotides, size to twelve nucleotides or six to ten nucleotides in length, in some embodiments.

In some embodiments, the gap region of the gapmer oligonucleotides may contain modified nucleotides known to be acceptable for efficient RNase H action in addition to DNA nucleotides, such as C4′-substituted nucleotides, acyclic nucleotides, and arabino-configured nucleotides. In some embodiments, the gap region comprises one or more unmodified internucleosides. In some embodiments, one or both flanking regions each independently comprise one or more phosphorothioate internucleoside linkages (e.g., phosphorothioate internucleoside linkages or other linkages) between at least two, at least three, at least four, at least five or more nucleotides. In some embodiments, the gap region and two flanking regions each independently comprise modified internucleoside linkages (e.g., phosphorothioate internucleoside linkages or other linkages) between at least two, at least three, at least four, at least five or more nucleotides.

A gapmer may be produced using appropriate methods. Representative U.S. patents, U.S. patent publications, and PCT publications that teach the preparation of gapmers include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; 5,898,031; 7,432,250; and 7,683,036; U.S. patent publication Nos. US20090286969, US20100197762, and US20110112170; and PCT publication Nos. WO2008049085 and WO2009090182, each of which is herein incorporated by reference in its entirety.

i. Mixmers

In some embodiments, an oligonucleotide described herein may be a mixmer or comprise a mixmer sequence pattern. In general, mixmers are oligonucleotides that comprise both naturally and non-naturally occurring nucleotides or comprise two different types of non-naturally occurring nucleotides typically in an alternating pattern. Mixmers generally have higher binding affinity than unmodified oligonucleotides and may be used to specifically bind a target molecule, e.g., to block a binding site on the target molecule. Generally, mixmers do not recruit an RNAse to the target molecule and thus do not promote cleavage of the target molecule. Such oligonucleotides that are incapable of recruiting RNAse H have been described, for example, see WO2007/112754 or WO2007/112753.

In some embodiments, the mixmer comprises or consists of a repeating pattern of nucleotide analogues and naturally occurring nucleotides, or one type of nucleotide analogue and a second type of nucleotide analogue. However, a mixmer need not comprise a repeating pattern and may instead comprise any arrangement of modified nucleotides and naturally occurring nucleotides or any arrangement of one type of modified nucleotide and a second type of modified nucleotide. The repeating pattern, may, for instance be every second or every third nucleotide is a modified nucleotide, such as LNA, and the remaining nucleotides are naturally occurring nucleotides, such as DNA, or are a 2′ substituted nucleotide analogue such as 2′MOE or 2′ fluoro analogues, or any other modified nucleotide described herein. It is recognized that the repeating pattern of modified nucleotide, such as LNA units, may be combined with modified nucleotide at fixed positions—e.g. at the 5′ or 3′ termini.

In some embodiments, a mixmer does not comprise a region of more than 5, more than 4, more than 3, or more than 2 consecutive naturally occurring nucleotides, such as DNA nucleotides. In some embodiments, the mixmer comprises at least a region consisting of at least two consecutive modified nucleotides, such as at least two consecutive LNAs. In some embodiments, the mixmer comprises at least a region consisting of at least three consecutive modified nucleotide units, such as at least three consecutive LNAs.

In some embodiments, the mixmer does not comprise a region of more than 7, more than 6, more than 5, more than 4, more than 3, or more than 2 consecutive nucleotide analogues, such as LNAs. In some embodiments, LNA units may be replaced with other nucleotide analogues, such as those referred to herein.

Mixmers may be designed to comprise a mixture of affinity enhancing modified nucleotides, such as in non-limiting example LNA nucleotides and 2′-O-methyl nucleotides. In some embodiments, a mixmer comprises modified internucleoside linkages (e.g., phosphorothioate internucleoside linkages or other linkages) between at least two, at least three, at least four, at least five or more nucleotides.

A mixmer may be produced using any suitable method. Representative U.S. patents, U.S. patent publications, and PCT publications that teach the preparation of mixmers include U.S. patent publication Nos. US20060128646, US20090209748, US20090298916, US20110077288, and US20120322851, and U.S. Pat. No. 7,687,617.

In some embodiments, a mixmer comprises one or more morpholino nucleotides. For example, in some embodiments, a mixmer may comprise morpholino nucleotides mixed (e.g., in an alternating manner) with one or more other nucleotides (e.g., DNA, RNA nucleotides) or modified nucleotides (e.g., LNA, 2′-O-Methyl nucleotides).

In some embodiments, mixmers are useful for splice correcting or exon skipping, for example, as reported in Touznik A., et al., LNA/DNA mixmer-based antisense oligonucleotides correct alternative splicing of the SMN2 gene and restore SMN protein expression in type 1 SMA fibroblasts Scientific Reports, volume 7, Article number: 3672 (2017), Chen S. et al., Synthesis of a Morpholino Nucleic Acid (MNA)-Uridine Phosphoramidite, and Exon Skipping Using MNA/2′-O-Methyl Mixmer Antisense Oligonucleotide, Molecules 2016, 21, 1582, the contents of each which are incorporated herein by reference.

j. RNA Interference (RNAi)

In some embodiments, oligonucleotides provided herein may be in the form of small interfering RNAs (siRNA), also known as short interfering RNA or silencing RNA. SiRNA, is a class of RNA molecules, typically about 20-25 base pairs in length that target nucleic acids (e.g., mRNAs) for degradation via the RNA interference (RNAi) pathway in cells. Specificity of siRNA molecules may be determined by the binding of the antisense strand of the molecule to its target RNA. Effective siRNA molecules are generally less than 30 to 35 base pairs in length to prevent the triggering of non-specific RNA interference pathways in the cell via the interferon response, although longer siRNA can also be effective.

Following selection of an appropriate target RNA sequence, siRNA molecules that comprise a nucleotide sequence complementary to all or a portion of the target sequence, i.e. an antisense sequence, can be designed and prepared using appropriate methods (see, e.g., PCT Publication Number WO 2004/016735; and U.S. Patent Publication Nos. 2004/0077574 and 2008/0081791).

The siRNA molecule can be double stranded (i.e. a dsRNA molecule comprising an antisense strand and a complementary sense strand) or single-stranded (i.e. a ssRNA molecule comprising just an antisense strand). The siRNA molecules can comprise a duplex, asymmetric duplex, hairpin or asymmetric hairpin secondary structure, having self-complementary sense and antisense strands.

Double-stranded siRNA may comprise RNA strands that are the same length or different lengths. Double-stranded siRNA molecules can also be assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating RNAi. Small hairpin RNA (shRNA) molecules thus are also contemplated herein. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in addition or removal of one, two, three or more nucleotides from the 3′ end and/or the 5′ end of either or both strands). A spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end and/or the 5′ end of either or both strands). A spacer sequence may be an unrelated nucleotide sequence that is situated between two complementary nucleotide sequence regions which, when annealed into a double-stranded nucleic acid, comprise a shRNA.

The overall length of the siRNA molecules can vary from about 14 to about 100 nucleotides depending on the type of siRNA molecule being designed. Generally, between about 14 and about 50 of these nucleotides are complementary to the RNA target sequence, i.e. constitute the specific antisense sequence of the siRNA molecule. For example, when the siRNA is a double- or single-stranded siRNA, the length can vary from about 14 to about 50 nucleotides, whereas when the siRNA is a shRNA or circular molecule, the length can vary from about 40 nucleotides to about 100 nucleotides.

An siRNA molecule may comprise a 3′ overhang at one end of the molecule. The other end may be blunt-ended or have also an overhang (5′ or 3′). When the siRNA molecule comprises an overhang at both ends of the molecule, the length of the overhangs may be the same or different. In one embodiment, the siRNA molecule of the present disclosure comprises 3′ overhangs of about 1 to about 3 nucleotides on both ends of the molecule.

k. microRNA (miRNAs)

In some embodiments, an oligonucleotide may be a microRNA (miRNA). MicroRNAs (referred to as “miRNAs”) are small non-coding RNAs, belonging to a class of regulatory molecules that control gene expression by binding to complementary sites on a target RNA transcript. Typically, miRNAs are generated from large RNA precursors (termed pri-miRNAs) that are processed in the nucleus into approximately 70 nucleotide pre-miRNAs, which fold into imperfect stem-loop structures. These pre-miRNAs typically undergo an additional processing step within the cytoplasm where mature miRNAs of 18-25 nucleotides in length are excised from one side of the pre-miRNA hairpin by an RNase III enzyme, Dicer.

As used herein, miRNAs including pri-miRNA, pre-miRNA, mature miRNA or fragments of variants thereof that retain the biological activity of mature miRNA. In one embodiment, the size range of the miRNA can be from 21 nucleotides to 170 nucleotides. In one embodiment the size range of the miRNA is from 70 to 170 nucleotides in length. In another embodiment, mature miRNAs of from 21 to 25 nucleotides in length can be used.

l. Aptamers

In some embodiments, oligonucleotides provided herein may be in the form of aptamers. Generally, in the context of molecular payloads, aptamer is any nucleic acid that binds specifically to a target, such as a small molecule, protein, nucleic acid in a cell. In some embodiments, the aptamer is a DNA aptamer or an RNA aptamer. In some embodiments, a nucleic acid aptamer is a single-stranded DNA or RNA (ssDNA or ssRNA). It is to be understood that a single-stranded nucleic acid aptamer may form helices and/or loop structures. The nucleic acid that forms the nucleic acid aptamer may comprise naturally occurring nucleotides, modified nucleotides, naturally occurring nucleotides with hydrocarbon linkers (e.g., an alkylene) or a polyether linker (e.g., a PEG linker) inserted between one or more nucleotides, modified nucleotides with hydrocarbon or PEG linkers inserted between one or more nucleotides, or a combination of thereof. Exemplary publications and patents describing aptamers and method of producing aptamers include, e.g., Lorsch and Szostak, 1996; Jayasena, 1999; U.S. Pat. Nos. 5,270,163; 5,567,588; 5,650,275; 5,670,637; 5,683,867; 5,696,249; 5,789,157; 5,843,653; 5,864,026; 5,989,823; 6,569,630; 8,318,438 and PCT application WO 99/31275, each incorporated herein by reference.

m. Ribozymes

In some embodiments, oligonucleotides provided herein may be in the form of a ribozyme. A ribozyme (ribonucleic acid enzyme) is a molecule, typically an RNA molecule, that is capable of performing specific biochemical reactions, similar to the action of protein enzymes. Ribozymes are molecules with catalytic activities including the ability to cleave at specific phosphodiester linkages in RNA molecules to which they have hybridized, such as mRNAs, RNA-containing substrates, lncRNAs, and ribozymes, themselves.

Ribozymes may assume one of several physical structures, one of which is called a “hammerhead.” A hammerhead ribozyme is composed of a catalytic core containing nine conserved bases, a double-stranded stem and loop structure (stem-loop II), and two regions complementary to the target RNA flanking regions the catalytic core. The flanking regions enable the ribozyme to bind to the target RNA specifically by forming double-stranded stems I and III. Cleavage occurs in cis (i.e., cleavage of the same RNA molecule that contains the hammerhead motif) or in trans (cleavage of an RNA substrate other than that containing the ribozyme) next to a specific ribonucleotide triplet by a transesterification reaction from a 3′,5′-phosphate diester to a 2′,3′-cyclic phosphate diester. Without wishing to be bound by theory, it is believed that this catalytic activity requires the presence of specific, highly conserved sequences in the catalytic region of the ribozyme.

Modifications in ribozyme structure have also included the substitution or replacement of various non-core portions of the molecule with non-nucleotidic molecules. For example, Benseler et al. (J. Am. Chem. Soc. (1993) 115:8483-8484) disclosed hammerhead-like molecules in which two of the base pairs of stem II, and all four of the nucleotides of loop II were replaced with non-nucleoside linkers based on hexaethylene glycol, propanediol, bis(triethylene glycol) phosphate, tris(propanediol)bisphosphate, or bis(propanediol) phosphate. Ma et al. (Biochem. (1993) 32:1751-1758; Nucleic Acids Res. (1993) 21:2585-2589) replaced the six nucleotide loop of the TAR ribozyme hairpin with non-nucleotidic, ethylene glycol-related linkers. Thomson et al. (Nucleic Acids Res. (1993) 21:5600-5603) replaced loop II with linear, non-nucleotidic linkers of 13, 17, and 19 atoms in length.

Ribozyme oligonucleotides can be prepared using well known methods (see, e.g., PCT Publications WO9118624; WO9413688; WO9201806; and WO 92/07065; and U.S. Pat. Nos. 5,436,143 and 5,650,502) or can be purchased from commercial sources (e.g., US Biochemicals) and, if desired, can incorporate nucleotide analogs to increase the resistance of the oligonucleotide to degradation by nucleases in a cell. The ribozyme may be synthesized in any known manner, e.g., by use of a commercially available synthesizer produced, e.g., by Applied Biosystems, Inc. or Milligen. The ribozyme may also be produced in recombinant vectors by conventional means. See, Molecular Cloning: A Laboratory Manual, Cold Spring Harbor Laboratory (Current edition). The ribozyme RNA sequences maybe synthesized conventionally, for example, by using RNA polymerases such as T7 or SP6.

n. Guide Nucleic Acids

In some embodiments, oligonucleotides are guide nucleic acid, e.g., guide RNA (gRNA) molecules. Generally, a guide RNA is a short synthetic RNA composed of (1) a scaffold sequence that binds to a nucleic acid programmable DNA binding protein (napDNAbp), such as Cas9, and (2) a nucleotide spacer portion that defines the DNA target sequence (e.g., genomic DNA target) to which the gRNA binds in order to bring the nucleic acid programmable DNA binding protein in proximity to the DNA target sequence. In some embodiments, the napDNAbp is a nucleic acid-programmable protein that forms a complex with (e.g., binds or associates with) one or more RNA(s) that targets the nucleic acid-programmable protein to a target DNA sequence (e.g., a target genomic DNA sequence). In some embodiments, a nucleic acid-programmable nuclease, when in a complex with an RNA, may be referred to as a nuclease:RNA complex. Guide RNAs can exist as a complex of two or more RNAs, or as a single RNA molecule.

Guide RNAs (gRNAs) that exist as a single RNA molecule may be referred to as single-guide RNAs (sgRNAs), though gRNA is also used to refer to guide RNAs that exist as either single molecules or as a complex of two or more molecules. Typically, gRNAs that exist as a single RNA species comprise two domains: (1) a domain that shares homology to a target nucleic acid (i.e., directs binding of a Cas9 complex to the target); and (2) a domain that binds a Cas9 protein. In some embodiments, domain (2) corresponds to a sequence known as a tracrRNA and comprises a stem-loop structure. In some embodiments, domain (2) is identical or homologous to a tracrRNA as provided in Jinek et al., Science 337:816-821 (2012), the entire contents of which is incorporated herein by reference.

In some embodiments, a gRNA comprises two or more of domains (1) and (2), and may be referred to as an extended gRNA. For example, an extended gRNA will bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example, Cas9 (Csn1) from Streptococcus pyogenes (see, e.g., “Complete genome sequence of an M1 strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference.

o. Splice Altering Oligonucleotides

In some embodiments, an oligonucleotide (e.g., an antisense oligonucleotide including a morpholino) of the present disclosure target splicing. In some embodiments, the oligonucleotide targets splicing by inducing exon skipping and restoring the reading frame within a gene. As a non-limiting example, the oligonucleotide may induce skipping of an exon encoding a frameshift mutation and/or an exon that encodes a premature stop codon. In some embodiments, an oligonucleotide may induce exon skipping by blocking spliceosome recognition of a splice site. In some embodiments, exon skipping results in a truncated but functional protein compared to the reference protein (e.g., truncated but functional DMD protein as described below). In some embodiments, the oligonucleotide promotes inclusion of a particular exon (e.g., exon 7 of the SMN2 gene described below). In some embodiments, an oligonucleotide may induce inclusion of an exon by targeting a splice site inhibitory sequence. RNA splicing has been implicated in muscle diseases, including Duchenne muscular dystrophy (DMD) and spinal muscular atrophy (SMA).

Alterations (e.g., deletions, point mutations, and duplications) in the gene encoding dystrophin (DMD) cause DMD. These alterations can lead to frameshift mutations and/or nonsense mutations. In some embodiments, an oligonucleotide of the present disclosure promotes skipping of one or more DMD exons (e.g., exon 8, exon 43, exon 44, exon 45, exon 50, exon 51, exon 52, exon 53, and/or exon 55) and results in a functional truncated protein. See, e.g., U.S. Pat. No. 8,486,907 published on Jul. 16, 2013 and U.S. 20140275212 published on Sep. 18, 2014.

In SMA, there is loss of functional SMN1. Although the SMN2 gene is a paralog to SMN1, alternative splicing of the SMN2 gene predominantly leads to skipping of exon 7 and subsequent production of a truncated SMN protein that cannot compensate for SMN1 loss. In some embodiments, an oligonucleotide of the present disclosure promotes inclusion of SMN2 exon 7. In some embodiments, an oligonucleotide is an antisense oligonucleotide that targets SMN2 splice site inhibitory sequences (see, e.g., U.S. Pat. No. 7,838,657, which was published on Nov. 23, 2010).

p. Multimers

In some embodiments, molecular payloads may comprise multimers (e.g., concatemers) of 2 or more oligonucleotides connected by a linker. In this way, in some embodiments, the oligonucleotide loading of a complex can be increased beyond the available linking sites on a targeting agent (e.g., available thiol sites on an antibody) or otherwise tuned to achieve a particular payload loading content. Oligonucleotides in a multimer can be the same or different (e.g., targeting different genes or different sites on the same gene or products thereof).

In some embodiments, multimers comprise 2 or more oligonucleotides linked together by a cleavable linker. However, in some embodiments, multimers comprise 2 or more oligonucleotides linked together by a non-cleavable linker. In some embodiments, a multimer comprises 2, 3, 4, 5, 6, 7, 8, 9, 10 or more oligonucleotides linked together. In some embodiments, a multimer comprises 2 to 5, 2 to 10 or 4 to 20 oligonucleotides linked together.

In some embodiments, a multimer comprises 2 or more oligonucleotides linked end-to-end (in a linear arrangement). In some embodiments, a multimer comprises 2 or more oligonucleotides linked end-to-end via an oligonucleotide-based linker (e.g., poly-dT linker, an abasic linker). In some embodiments, a multimer comprises a 5′ end of one oligonucleotide linked to a 3′ end of another oligonucleotide. In some embodiments, a multimer comprises a 3′ end of one oligonucleotide linked to a 3′ end of another oligonucleotide. In some embodiments, a multimer comprises a 5′ end of one oligonucleotide linked to a 5′ end of another oligonucleotide. Still, in some embodiments, multimers can comprise a branched structure comprising multiple oligonucleotides linked together by a branching linker.

Further examples of multimers that may be used in the complexes provided herein are disclosed, for example, in US Patent Application Number 2015/0315588 A1, entitled Methods of delivering multiple targeting oligonucleotides to a cell using cleavable linkers, which was published on Nov. 5, 2015; US Patent Application Number 2015/0247141 A1, entitled Multimeric Oligonucleotide Compounds, which was published on Sep. 3, 2015, US Patent Application Number US 2011/0158937 A1, entitled Immunostimulatory Oligonucleotide Multimers, which was published on Jun. 30, 2011; and U.S. Pat. No. 5,693,773, entitled Triplex-Forming Antisense Oligonucleotides Having Abasic Linkers Targeting Nucleic Acids Comprising Mixed Sequences Of Purines And Pyrimidines, which issued on Dec. 2, 1997, the contents of each of which are incorporated herein by reference in their entireties.

In certain embodiments, R is a therapeutic moiety. In certain embodiments, R is a molecular payload. In some embodiments, the molecular payload is a small molecule. In some embodiments, the molecular payload is an oligonucleotide. In some embodiments, the molecular payload is a charge-neutral oligonucleotide. In some embodiments, the oligonucleotide is a single stranded oligonucleotide (e.g., a charge-neutral single stranded oligonucleotide, a charged-single stranded oligonucleotide). In some embodiments, the charge-neutral stranded oligonucleotide is an antisense oligonucleotide. In some embodiments, the charge-neutral oligo nucleotide is a phosphorodiamidate morpholino oligomer (PMO). In some embodiments, the charge-neutral oligonucleotide is a peptide nucleic acid (PNA). In some embodiments, the molecular payload is a charged oligonucleotide. In some embodiments, the charged oligonucleotide comprises a phosphodiester backbone that has a net negative charge at physiological pH (e.g., pH 7.35-pH 7.45). In some embodiments, the charged oligonucleotide comprises a phosphothioate backbone that has a net negative charge at physiological pH (e.g., pH 7.35-pH 7.45). In some embodiments, when R is an oligonucleotide, L³ is covalently linked to the 5′ end of the oligonucleotide (e.g., PMO). In some embodiments, when R is an oligonucleotide, L³ is covalently linked to the 3′ end of the oligonucleotide (e.g., PMO).

In some embodiments, the charge-neutral oligonucleotide (e.g., PMO) is 10-50 (e.g., 10-50, 10-40, 10-30, 10-20, 20-50, 20-40, 20-30, 30-50, 30-40, or 40-50 nucleotides in length). In some embodiments, the charge-neutral oligonucleotide (e.g., PMO) is 15-30 (e.g., 15-30, 15-25, 15-20, 20-30, 20-25, or 25-30) nucleotides in length. In some embodiments, the charge-neutral oligonucleotide (e.g., PMO) is 15, 16, 17, 18, 19, 20, 21, 22, 23, 24, 25, 26, 27, 28, 29, or 30 nucleotides in length. In some embodiments, the charge-neutral oligonucleotide (e.g., PMO) is 30 nucleotides in length.

In some embodiments, the charged oligonucleotide (e.g., an oligonucleotide comprising a phosphothioate backbone, an oligonucleotide comprising a phosphodiester backbone) is 10-50 (e.g., 10-50, 10-40, 10-30, 10-20, 20-50, 20-40, 20-30, 30-50, 30-40, or 40-50 nucleotides in length). In some embodiments, the charged oligonucleotide (e.g., an oligonucleotide comprising a phosphothioate backbone, an oligonucleotide comprising a phosphodiester backbone) is 15-30 (e.g., 15-30, 15-25, 15-20, 20-30, 20-25, or 25-30) nucleotides in length. In some embodiments, the charged oligonucleotide (e.g., an oligonucleotide comprising a phosphothioate backbone, an oligonucleotide comprising a phosphodiester backbone) is 15, 16, 17, 18, 19, 20, 21, 22, 23, 24, 25, 26, 27, 28, 29, or 30 nucleotides in length. In some embodiments, the charged neutral oligonucleotide (e.g., an oligonucleotide comprising a phosphothioate backbone, an oligonucleotide comprising a phosphodiester backbone) is 30 nucleotides in length.

### II. Further Embodiments of Formula (I)

In certain embodiments, the compound of Formula (I) is a compound of Formula (I-a):

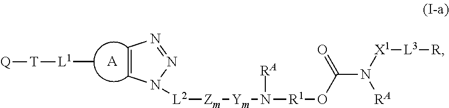

or a pharmaceutically acceptable salt, tautomer, stereoisomer, or isotopically enriched derivative thereof, wherein Q, T, L¹, A, L², Z, Y, R^{A}, R¹, L³, and R are as defined herein.

In certain embodiments, the compound of Formula (I) is a compound of Formula (I-b):

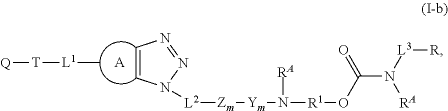

or a pharmaceutically acceptable salt, tautomer, stereoisomer, or isotopically enriched derivative thereof, wherein Q, T, L¹, A, L², Z, Y, R^{A}, R¹, L³, and R are as defined herein.

In certain embodiments, the compound of Formula (I) is a compound of Formula (I-c):

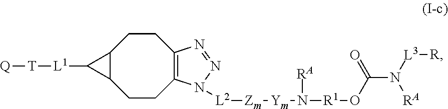

or a pharmaceutically acceptable salt, tautomer, stereoisomer, or isotopically enriched derivative thereof, wherein Q, T, L¹, L², Z, Y, m, R^{A}, R¹, L³, and R are as defined herein.

In certain embodiments, the compound of Formula (I) is a compound of Formula (I-d):

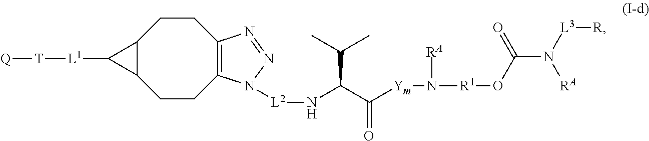

or a pharmaceutically acceptable salt, tautomer, stereoisomer, or isotopically enriched derivative thereof, wherein Q, T, L¹, L², Y, m, R^{A}, R¹, L³, and R are as defined herein.

In certain embodiments, the compound of Formula (I) is a compound of Formula (I-e):

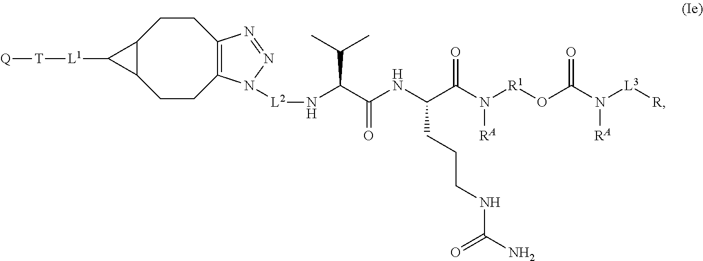

or a pharmaceutically acceptable salt, tautomer, stereoisomer, or isotopically enriched derivative thereof, wherein Q, T, L¹, L², R^{A}, R¹, L³, and R are as defined herein.

In certain embodiments, the compound of Formula (I) is a compound of Formula (I-f):

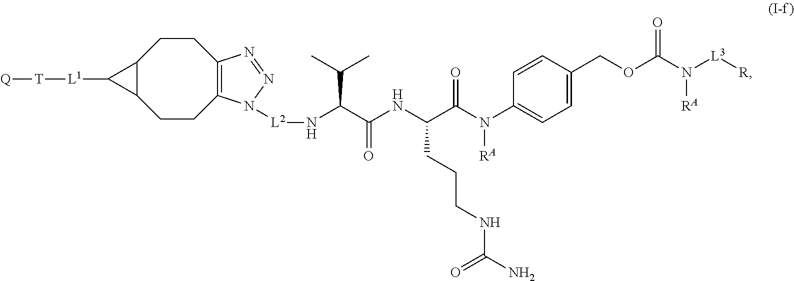

or a pharmaceutically acceptable salt, tautomer, stereoisomer, or isotopically enriched derivative thereof, wherein Q, T, L¹, L², R^{A}, L³, and R are as defined herein.

In certain embodiments, the compound of Formula (I) is a compound of Formula (I-g):

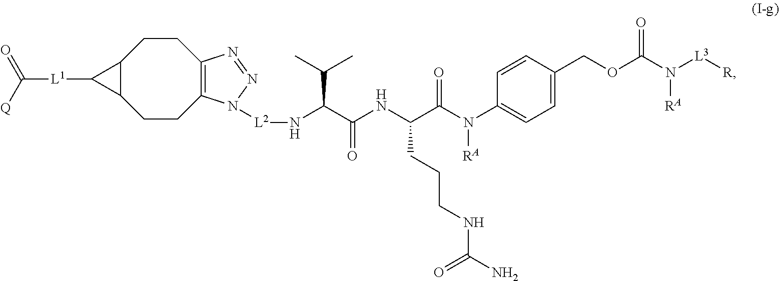

or a pharmaceutically acceptable salt, tautomer, stereoisomer, or isotopically enriched derivative thereof, wherein Q, T, L¹, L², R^{A}, L³, and R are as defined herein.

In certain embodiments, the compound of Formula (I) is a compound of Formula (I-h):

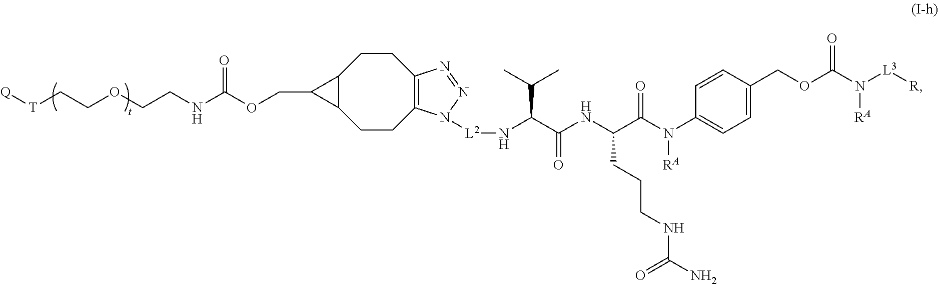

or a pharmaceutically acceptable salt, tautomer, stereoisomer, or isotopically enriched derivative thereof, wherein Q, T, t, L², R^{A}, L³, and R are as defined herein.

In certain embodiments, the compound of Formula (I) is a compound of Formula (I-i):

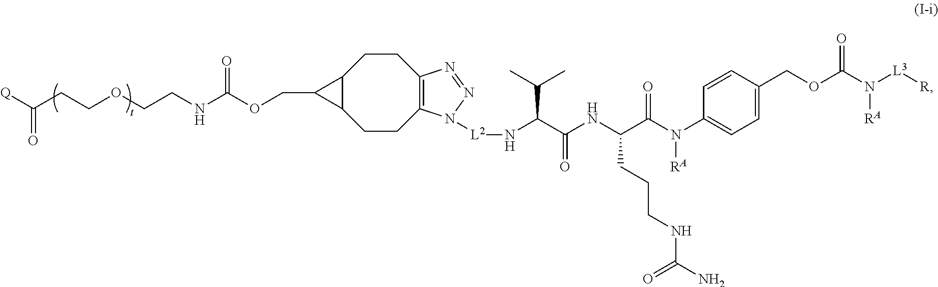

or a pharmaceutically acceptable salt, tautomer, stereoisomer, or isotopically enriched derivative thereof, wherein Q, t, L², R^{A}, L³, and R are as defined herein.

In certain embodiments, the compound of Formula (I) is a compound of Formula (I-j):

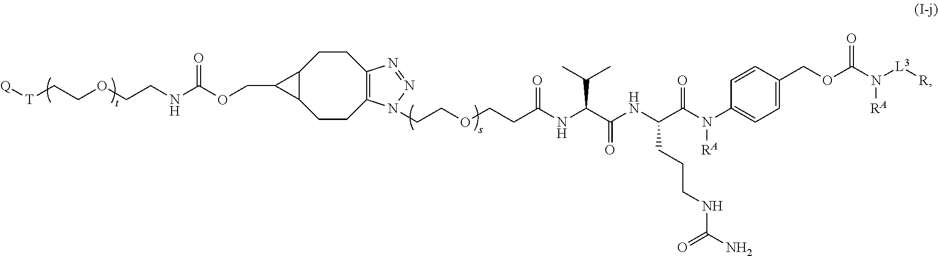

or a pharmaceutically acceptable salt, tautomer, stereoisomer, or isotopically enriched derivative thereof, wherein Q, T, R^{A}, L³, R, t, and s are as defined herein.

In certain embodiments, the compound of Formula (I) is a compound of Formula (I-j-1):

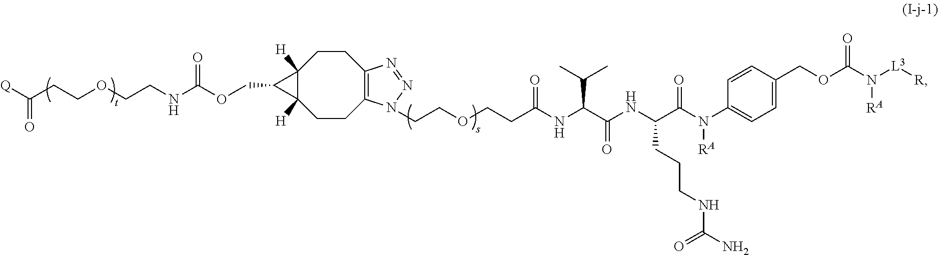

or a pharmaceutically acceptable salt, tautomer, stereoisomer, or isotopically enriched derivative thereof, wherein Q, R^{A}, L³, R, t, and s are as defined herein.

In certain embodiments, the compound of Formula (I) is a compound of Formula (I-j-2):

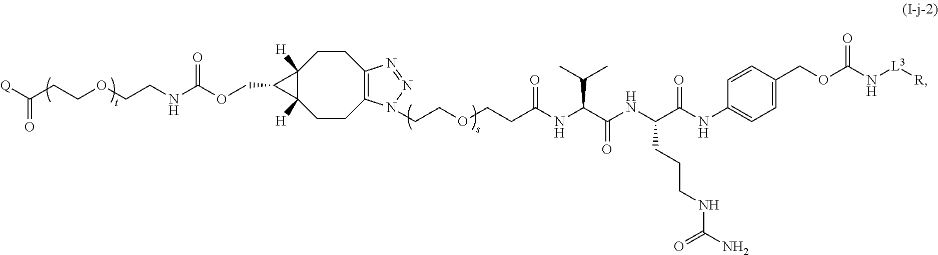

or a pharmaceutically acceptable salt, tautomer, stereoisomer, or isotopically enriched derivative thereof, wherein Q, L³, R, t, and s are as defined herein.

In certain embodiments, the compound of Formula (I) is a compound of Formula (I-j-3):

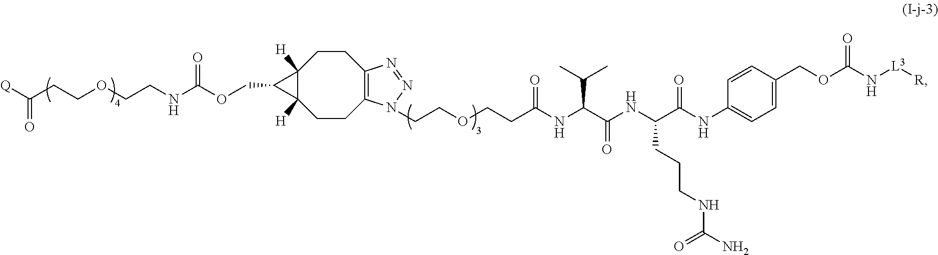

or a pharmaceutically acceptable salt, tautomer, stereoisomer, or isotopically enriched derivative thereof, wherein Q, L³, R, t, and s are as defined herein.

In certain embodiments, the compound of Formula (I) is a compound of Formula (I-k):

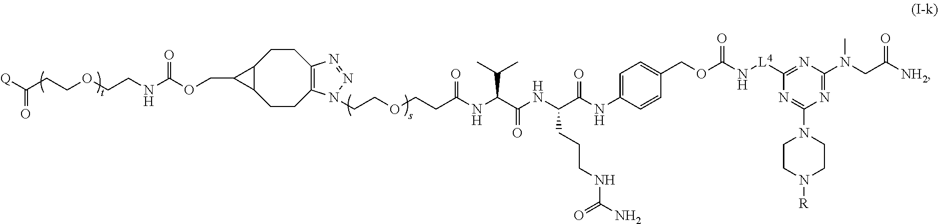

or a pharmaceutically acceptable salt, tautomer, stereoisomer, or isotopically enriched derivative thereof, wherein Q, L⁴, R, t, and s are as defined herein.

In certain embodiments, the compound of Formula (I) is a compound of Formula

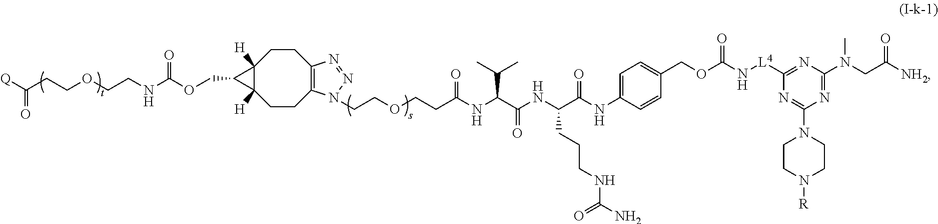

or a pharmaceutically acceptable salt, tautomer, stereoisomer, or isotopically enriched derivative thereof, wherein Q, L⁴, R, t, and s are as defined herein.

In certain embodiments, the compound of Formula (I) is a compound of Formula

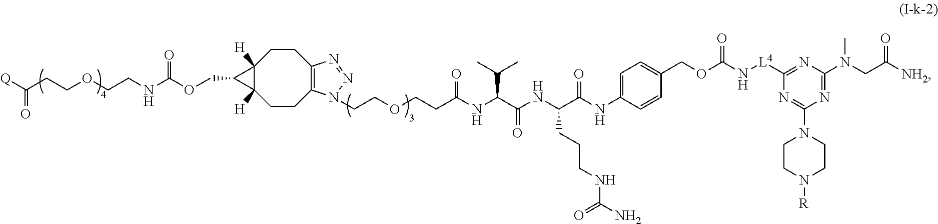

or a pharmaceutically acceptable salt, tautomer, stereoisomer, or isotopically enriched derivative thereof, wherein Q, L⁴, and R are as defined herein.

In certain embodiments, the compound of Formula (I) is a compound of Formula (I-m):

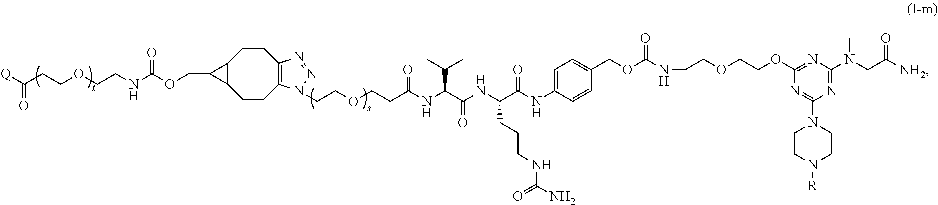

or a pharmaceutically acceptable salt, tautomer, stereoisomer, or isotopically enriched derivative thereof, wherein Q, R, t, and s are as defined herein.

In certain embodiments, the compound of Formula (I) is a compound of Formula (I-m-1):

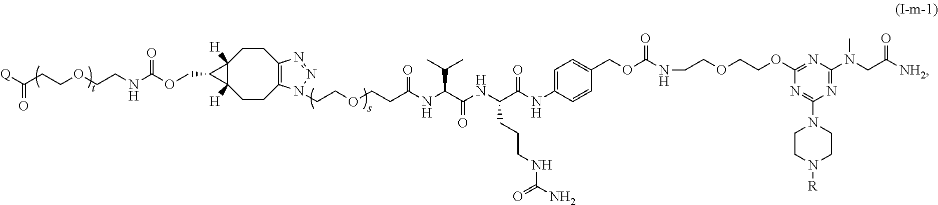

or a pharmaceutically acceptable salt, tautomer, stereoisomer, or isotopically enriched derivative thereof, wherein Q, R, t, and s are as defined herein.

In certain embodiments, the compound of Formula (I) is a compound of Formula (I-m-2):

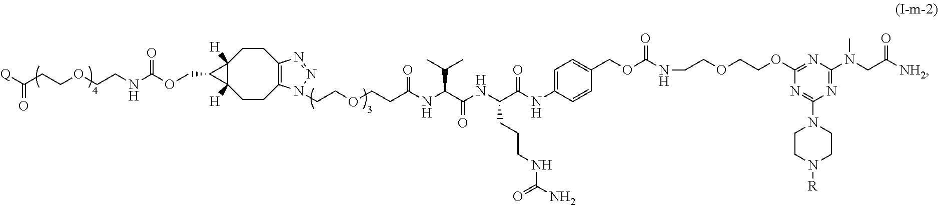

or a pharmaceutically acceptable salt, tautomer, stereoisomer, or isotopically enriched derivative thereof, wherein Q and R are as defined herein.

### III. Methods of Preparing the Compounds

Also disclosed herein are methods of preparing compounds of Formula (I).

a. Method 1

In one aspect, disclosed is a method of preparing a compound of Formula (I):

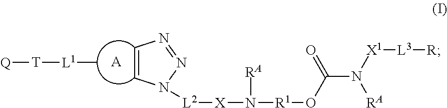

or a salt thereof, the method comprising coupling a targeting agent (Q) with a compound of Formula (II):

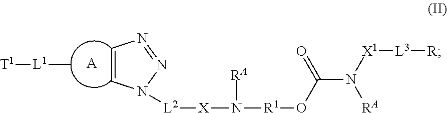

or a salt thereof, to provide a compound of Formula (I), wherein T, L¹, A, L², X, R^{A}, R¹, X¹, L³, and R are as defined herein;
T₁ is

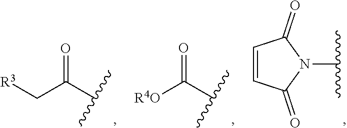

S═C═N—, or

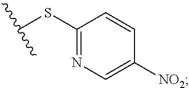 [SMILES: *Sc1ccc([N+](=O)[O-])cn1]

R³ is a leaving group; and
R⁴ is hydrogen; substituted or unsubstituted alkyl; substituted or unsubstituted heterocyclyl; substituted or unsubstituted aryl; substituted or unsubstituted heteroaryl; or an oxygen protecting group.

In certain embodiments, the coupling of a targeting agent (Q) with a compound of Formula (II) is performed using 1, 2, 3, 4, 5, 6, 7, 8, 9, or 10 molar equivalents of Formula (II) relative to the targeting agent. In certain embodiments, the coupling of a targeting agent (Q) with a compound of Formula (II) is performed using 1 molar equivalent of Formula (II) relative to the targeting agent. In certain embodiments, the coupling of a targeting agent (Q) with a compound of Formula (II) is performed using 2 molar equivalent of Formula (II) relative to the targeting agent. In certain embodiments, the coupling of a targeting agent (Q) with a compound of Formula (II) is performed using 3 molar equivalent of Formula (II) relative to the targeting agent. In certain embodiments, the coupling of a targeting agent (Q) with a compound of Formula (II) is performed using 4 molar equivalent of Formula (II) relative to the targeting agent. In certain embodiments, the coupling of a targeting agent (Q) with a compound of Formula (II) is performed using 5 molar equivalent of Formula (II) relative to the targeting agent. In certain embodiments, the coupling of a targeting agent (Q) with a compound of Formula (II) is performed using 6 molar equivalent of Formula (II) relative to the targeting agent. In certain embodiments, the coupling of a targeting agent (Q) with a compound of Formula (II) is performed using 7 molar equivalent of Formula (II) relative to the targeting agent. In certain embodiments, the coupling of a targeting agent (Q) with a compound of Formula (II) is performed using 8 molar equivalent of Formula (II) relative to the targeting agent. In certain embodiments, the coupling of a targeting agent (Q) with a compound of Formula (II) is performed using 9 molar equivalent of Formula (II) relative to the targeting agent. In certain embodiments, the coupling of a targeting agent (Q) with a compound of Formula (II) is performed using 10 molar equivalent of Formula (II) relative to the targeting agent.

In certain embodiments, the coupling of a targeting agent (Q) with a compound of Formula (II) results in formation of the compound of Formula (I) having an average ratio of molecular payload (R) to targeting agent (Q) of about 1 to about 4, about 1 to about 3, about 1 to about 2, about 2 to about 4, about 2 to about 3, or about 3 to about 4. In certain embodiments, the coupling of a targeting agent (Q) with a compound of Formula (II) results in formation of the compound of Formula (I) having an average ratio of molecular payload (R) to targeting agent (Q) of about 1, about 2, about 3, or about 4.

In certain embodiments, the coupling of a targeting agent (Q) with a compound of Formula (II) takes place in solvent comprising dimethyl acetamide or isopropyl alcohol. In certain embodiments, the solvent is or comprises isopropyl alcohol. In certain embodiments, the solvent is or comprises dimethyl acetamide. In certain embodiments, the coupling of a targeting agent (Q) with a compound of Formula (II) is performed at a pH of about 7.0 to about 8.5, about 7.0 to about 8.0, about 7.5 to about 8.5, or about 7.5 to about 8.0. In certain embodiments, the coupling of a targeting agent (Q) with a compound of Formula (II) is performed at a pH of about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, or about 8.5. In certain embodiments, the coupling of a targeting agent (Q) with a compound of Formula (II) further comprises adding a buffer having a pKa of about 7.0 to about 8.5, about 7.0 to about 8.0, about 7.5 to about 8.5, or about 7.5 to about 8.0. In certain embodiments, the coupling of a targeting agent (Q) with a compound of Formula (II) further comprises adding a buffer having a pKa of about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, or about 8.5. In certain embodiments, the buffer is HEPES (N-(2-Hydroxyethyl)piperazine-N′-(2-ethanesulfonic acid)). In certain embodiments, the buffer is EPPS (N-(2-Hydroxyethyl)piperazine-N′-(3-propanesulfonic acid)).

In certain embodiments, the method further comprises reacting a compound of Formula (III):

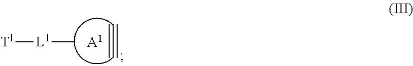

or a salt thereof, with a compound of Formula (IV):

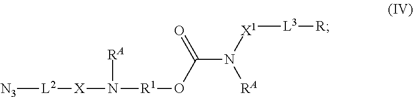

or a salt thereof, to provide a compound of Formula (II), or a salt thereof, wherein:
A¹ is substituted or unsubstituted carbocycle or substituted or unsubstituted heterocycle, or A¹ is absent and L¹ is bonded directly to one terminus of the alkyne.

In certain embodiments, the reacting of a compound of Formula (III) with a compound of Formula (IV) takes place in solvent comprising dimethyl acetamide or isopropyl alcohol. In certain embodiments, the reacting of a compound of Formula (III) with a compound of Formula (IV) takes place in solvent comprising dimethyl sulfoxide, acetonitrile, water, dimethyl acetamide, or isopropyl alcohol. In certain embodiments, the solvent is or comprises isopropyl alcohol. In certain embodiments, the solvent is or comprises dimethyl acetamide. In certain embodiments, the solvent is or comprises dimethyl sulfoxide. In certain embodiments, the solvent is or comprises acetonitrile. In certain embodiments, the solvent is or comprises water. In certain embodiments, the solvent is or comprises dimethyl acetamide and dimethyl sulfoxide. In certain embodiments, the solvent is or comprises dimethyl acetamide and water. In certain embodiments, the solvent is or comprises dimethyl acetamide, water, and acetonitrile.

In certain embodiments, the method further comprises purifying the compound of Formula (II). In certain embodiments, purifying the compound of Formula (II) comprises adding, to the reaction mixture comprising the completed reaction of the compound of Formula (III) with the compound of Formula (IV), a volume of a solvent that is at least 2, 3, 4, 5, 6, 7, 8, 9, or 10 times the amount of the total volume of the reaction mixture of the compound of Formula (III) with the compound of Formula (IV); and isolating the compound of Formula (II) from the reaction mixture. In certain embodiments, purifying the compound of Formula (II) comprises adding, to the reaction mixture comprising the completed reaction of the compound of Formula (III) with the compound of Formula (IV), a volume of a solvent that is at least 8 times the amount of the total volume of the reaction mixture of the compound of Formula (III) with the compound of Formula (IV); and isolating the compound of Formula (II) from the reaction mixture. In certain embodiments, purifying the compound of Formula (II) comprises adding, to the reaction mixture comprising the completed reaction of the compound of Formula (III) with the compound of Formula (IV), a volume of a solvent that is at least 3 times the amount of the total volume of the reaction mixture of the compound of Formula (III) with the compound of Formula (IV); and isolating the compound of Formula (II) from the reaction mixture. In certain embodiments, purifying the compound of Formula (II) comprises adding, to the completed reaction mixture of the compound of Formula (III) with the compound of Formula (IV), a volume of a solvent that is about 2, 3, 4, 5, 6, 7, 8, 9, or 10 times the amount of the total volume of the reaction mixture of the compound of Formula (III) with the compound of Formula (IV); and isolating the compound of Formula (II) from the reaction mixture. In certain embodiments, purifying the compound of Formula (II) comprises adding, to the reaction mixture comprising the completed reaction of the compound of Formula (III) with the compound of Formula (IV), a volume of a solvent that is about 8 times the amount of the total volume of the reaction mixture of the compound of Formula (III) with the compound of Formula (IV); and isolating the compound of Formula (II) from the reaction mixture. In certain embodiments, purifying the compound of Formula (II) comprises adding, to the reaction mixture comprising the completed reaction of the compound of Formula (III) with the compound of Formula (IV), a volume of a solvent that is about 3 times the amount of the total volume of the reaction mixture of the compound of Formula (III) with the compound of Formula (IV); and isolating the compound of Formula (II) from the reaction mixture.

In certain embodiments, the compound of Formula (II) is isolated as a solid.

In certain embodiments, the solvent added in the purification is acetone or isopropyl alcohol. In certain embodiments, the solvent added in the purification is acetone. In certain embodiments, the solvent added in the purification is isopropyl alcohol. In certain embodiments, the solvent added in the purification is cooled to a temperature that is below room temperature prior to adding the solvent. In certain embodiments, the solvent added in the purification is at a temperature that is equal to or below 30° C., 25° C., 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C. In certain embodiments, the solvent added in the purification is at a temperature of about 30° C., 25° C., 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C. In certain embodiments, the solvent added in the purification is at a temperature that is equal to or below room temperature. In certain embodiments, the solvent added in the purification is at room temperature. In certain embodiments, the solvent added in the purification is at a temperature that is equal to or below 0° C. In certain embodiments, the solvent added in the purification is at a temperature of about 0° C. In certain embodiments, the solvent added in the purification is at a temperature that is equal to or below −80° C. In certain embodiments, the solvent added in the purification is at a temperature of about −80° C.

In certain embodiments, purifying the compound of Formula (II) further comprises, after adding the purification solvent, cooling the reaction mixture to a temperature that is equal to or below room temperature. In certain embodiments, purifying the compound of Formula (II) further comprises, after adding the purification solvent, cooling the reaction mixture to a temperature that is equal to or below 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C. In certain embodiments, purifying the compound of Formula (II) further comprises, after adding the purification solvent, cooling the reaction mixture to a temperature of about 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C.

In certain embodiments, purifying the compound of Formula (II) further comprises adding, to the reaction mixture comprising the completed reaction of the compound of Formula (III) with the compound of Formula (IV), a volume of an aqueous solution of a salt that is less than 1, 0.9, 0.8, 0.7, 0.6, 0.5, 0.4, 0.3, 0.2, or 0.1 times the amount of the total volume of the reaction mixture of the compound of Formula (III) with the compound of Formula (IV). In certain embodiments, purifying the compound of Formula (II) further comprises adding, to the reaction mixture comprising the completed reaction of the compound of Formula (III) with the compound of Formula (IV), a volume of an aqueous solution of a salt that is about 1, 0.9, 0.8, 0.7, 0.6, 0.5, 0.4, 0.3, 0.2, or 0.1 times the amount of the total volume of the reaction mixture of the compound of Formula (III) with the compound of Formula (IV). In certain embodiments, purifying the compound of Formula (II) further comprises adding, to the reaction mixture comprising the completed reaction of the compound of Formula (III) with the compound of Formula (IV), a volume of an aqueous solution of a salt that is about 0.1 times the amount of the total volume of the reaction mixture of the compound of Formula (III) with the compound of Formula (IV). In certain embodiments, purifying the compound of Formula (II) further comprises adding, to the reaction mixture comprising the completed reaction of the compound of Formula (III) with the compound of Formula (IV), a volume of a 3M aqueous solution of a salt that is about 0.1 times the amount of the total volume of the reaction mixture of the compound of Formula (III) with the compound of Formula (IV). In certain embodiments, the salt is an alkali halide. In certain embodiments, the salt is LiCl, KCl, or NaCl. In certain embodiments, the salt is NaCl.

In certain embodiments wherein R is a charge-neutral oligonucleotide (e.g., phosphorodiamidate morpholino oligomer (PMO)), purifying the compound of Formula (II) comprises adding, to the reaction mixture comprising the completed reaction of the compound of Formula (III) with the compound of Formula (IV), a volume of a solvent that is about 8 times the amount of the total volume of the reaction mixture of the compound of Formula (III) with the compound of Formula (IV); and isolating the compound of Formula (II) from the reaction mixture. In certain embodiments wherein R is a charge-neutral oligonucleotide (e.g., phosphorodiamidate morpholino oligomer (PMO)), purifying the compound of Formula (II) comprises adding, to the reaction mixture comprising the completed reaction of the compound of Formula (III) with the compound of Formula (IV), a volume of acetone or isopropyl alcohol that is about 8 times the amount of the total volume of the reaction mixture of the compound of Formula (III) with the compound of Formula (IV); and isolating the compound of Formula (II) from the reaction mixture. In certain embodiments wherein R is a charge-neutral oligonucleotide (e.g., phosphorodiamidate morpholino oligomer (PMO)), purifying the compound of Formula (II) comprises adding, to the reaction mixture comprising the completed reaction of the compound of Formula (III) with the compound of Formula (IV), a volume of acetone or isopropyl alcohol that is about 8 times the amount of the total volume of the reaction mixture of the compound of Formula (III) with the compound of Formula (IV); and isolating the compound of Formula (II) from the reaction mixture; wherein the acetone or isopropyl alcohol is cooled to a temperature that is equal to or below 0° C. prior to adding the solvent. In certain embodiments wherein R is a charge-neutral oligonucleotide (e.g., phosphorodiamidate morpholino oligomer (PMO)), purifying the compound of Formula (II) comprises adding, to the reaction mixture comprising the completed reaction of the compound of Formula (III) with the compound of Formula (IV), a volume of acetone that is about 8 times the amount of the total volume of the reaction mixture of the compound of Formula (III) with the compound of Formula (IV); and isolating the compound of Formula (II) from the reaction mixture; wherein the acetone is cooled to a temperature that is equal to or below 0° C. prior to adding the solvent. In certain embodiments wherein R is a charge-neutral oligonucleotide (e.g., phosphorodiamidate morpholino oligomer (PMO)), purifying the compound of Formula (II) comprises adding, to the reaction mixture comprising the completed reaction of the compound of Formula (III) with the compound of Formula (IV), a volume of isopropyl alcohol that is about 8 times the amount of the total volume of the reaction mixture of the compound of Formula (III) with the compound of Formula (IV); and isolating the compound of Formula (II) from the reaction mixture; wherein the isopropyl alcohol is cooled to a temperature that is equal to or below 0° C. prior to adding the solvent.

In certain embodiments wherein R is a charged oligonucleotide, purifying the compound of Formula (II) comprises adding, to the reaction mixture comprising the completed reaction of the compound of Formula (III) with the compound of Formula (IV), a volume of a solvent that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of an aqueous solution of a salt that is about 0.1 times the amount of the total volume of the reaction mixture of the compound of Formula (III) with the compound of Formula (IV); and isolating the compound of Formula (II) from the reaction mixture. In certain embodiments wherein R is a charged oligonucleotide, purifying the compound of Formula (II) comprises adding, to the reaction mixture comprising the completed reaction of the compound of Formula (III) with the compound of Formula (IV), a volume of acetone or isopropyl alcohol that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of a 3M aqueous solution of a salt that is about 0.1 times the amount of the total volume of the reaction mixture of the compound of Formula (III) with the compound of Formula (IV); and isolating the compound of Formula (II) from the reaction mixture. In certain embodiments wherein R is a charged oligonucleotide, purifying the compound of Formula (II) comprises adding, to the reaction mixture comprising the completed reaction of the compound of Formula (III) with the compound of Formula (IV), a volume of acetone or isopropyl alcohol that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of a 3M aqueous solution of a salt that is about 0.1 times the amount of the total volume of the reaction mixture of the compound of Formula (III) with the compound of Formula (IV); and isolating the compound of Formula (II) from the reaction mixture; wherein the acetone or isopropyl alcohol is cooled to a temperature that is equal to or below 0° C. prior to adding the solvent. In certain embodiments wherein R is a charged oligonucleotide, purifying the compound of Formula (II) comprises adding, to the reaction mixture comprising the completed reaction of the compound of Formula (III) with the compound of Formula (IV), a volume of acetone that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of a 3M aqueous solution of NaCl that is about 0.1 times the amount of the total volume of the reaction mixture of the compound of Formula (III) with the compound of Formula (IV); and isolating the compound of Formula (II) from the reaction mixture; wherein the acetone is cooled to a temperature that is equal to or below 0° C. prior to adding the solvent. In certain embodiments wherein R is a charged oligonucleotide, purifying the compound of Formula (II) comprises adding, to the reaction mixture comprising the completed reaction of the compound of Formula (III) with the compound of Formula (IV), a volume of isopropyl alcohol that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of a 3M aqueous solution of NaCl that is about 0.1 times the amount of the total volume of the reaction mixture of the compound of Formula (III) with the compound of Formula (IV); and isolating the compound of Formula (II) from the reaction mixture; wherein the isopropyl alcohol is cooled to a temperature that is equal to or below 0° C. prior to adding the solvent.

In certain embodiments wherein R is a charged oligonucleotide, purifying the compound of Formula (II) comprises adding, to the reaction mixture comprising the completed reaction of the compound of Formula (III) with the compound of Formula (IV), a volume of acetone or isopropyl alcohol that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of a 3M aqueous solution of a salt that is about 0.1 times the amount of the total volume of the reaction mixture of the compound of Formula (III) with the compound of Formula (IV); cooling the reaction mixture to a temperature of about −80° C.; and isolating the compound of Formula (II) from the reaction mixture; wherein the acetone or isopropyl alcohol is cooled to a temperature that is equal to or below 0° C. prior to adding the solvent. In certain embodiments wherein R is a charged oligonucleotide, purifying the compound of Formula (II) comprises adding, to the reaction mixture comprising the completed reaction of the compound of Formula (III) with the compound of Formula (IV), a volume of acetone that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of a 3M aqueous solution of NaCl that is about 0.1 times the amount of the total volume of the reaction mixture of the compound of Formula (III) with the compound of Formula (IV); cooling the reaction mixture to a temperature of about −80° C.; and isolating the compound of Formula (II) from the reaction mixture; wherein the acetone is cooled to a temperature that is equal to or below 0° C. prior to adding the solvent. In certain embodiments wherein R is a charged oligonucleotide, purifying the compound of Formula (II) comprises adding, to the reaction mixture comprising the completed reaction of the compound of Formula (III) with the compound of Formula (IV), a volume of isopropyl alcohol that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of a 3M aqueous solution of NaCl that is about 0.1 times the amount of the total volume of the reaction mixture of the compound of Formula (III) with the compound of Formula (IV); cooling the reaction mixture to a temperature of about −80° C.; and isolating the compound of Formula (II) from the reaction mixture; wherein the isopropyl alcohol is cooled to a temperature that is equal to or below 0° C. prior to adding the solvent.

In certain embodiments, the method further comprises reacting a compound of Formula (V):

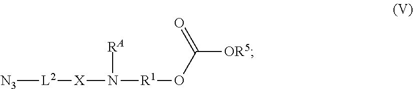

or a salt thereof, with a compound of Formula (VI):

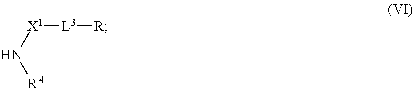

or a salt thereof, to provide a compound of Formula (IV), or a salt thereof, wherein:
R⁵ is substituted or unsubstituted aryl, or substituted or unsubstituted heteroaryl

In certain embodiments, the method further comprises conjugating group R (molecular payload) with a precursor of the compound of Formula (VI) to provide the compound of Formula (VI). In certain embodiments wherein R is a single-stranded oligonucleotide (e.g., PMO), the conjugating of R occurs by a suitable coupling reaction to provide the compound of Formula (VI). In certain embodiments, the oligonucleotide is coupled at its 5′ end. In certain embodiments wherein R is a double-stranded oligonucleotide (e.g., siRNA), the conjugating occurs by a suitable coupling reaction of one strand followed by an annealing of the second strand to provide the compound of Formula (VI). In certain embodiments, the oligonucleotide is coupled at its 5′ end.

In certain embodiments, the method further comprises reacting a compound of Formula (VII):

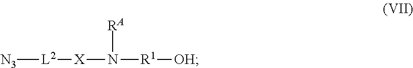

or a salt thereof, with a compound of Formula (VIII):

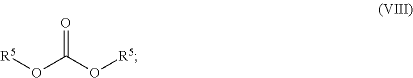

or a salt thereof, to provide a compound of Formula (V), or a salt thereof, wherein:
R⁵ is substituted or unsubstituted aryl, or substituted or unsubstituted heteroaryl.

In certain embodiments, the method further comprises reacting a compound of Formula (IX):

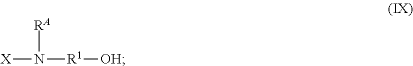

or a salt thereof, with a compound of Formula (X):

N₃-L²-LG (X);

or a salt thereof, to provide a compound of Formula (VII), or a salt thereof, wherein:
LG is a leaving group.

b. Method 2

In another aspect, disclosed is a method of preparing a compound of Formula (I):

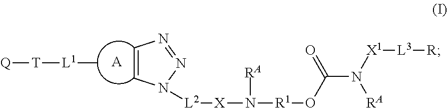

or a salt thereof, the method comprising reacting a compound of Formula (IV):

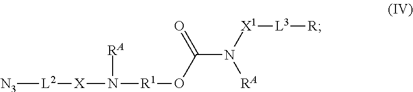

or a salt thereof, with a compound of Formula (B):

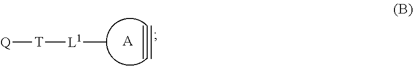

or a salt thereof, to provide a compound of Formula (I), wherein T, L¹, A, L², X, R^{A}, R¹, X¹, L³, and R are as defined herein; and
A¹ is substituted or unsubstituted carbocycle or substituted or unsubstituted heterocycle, or A¹ is absent and L¹ is bonded directly to one terminus of the alkyne.

In certain embodiments, the reacting of compound of Formula (IV) with a compound of Formula (B) is performed using 1, 2, 3, 4, 5, 6, 7, 8, 9, or 10 molar equivalents of Formula (IV) relative to the compound of Formula (B). In certain embodiments, the reacting of compound of Formula (IV) with a compound of Formula (B) is performed using 1 molar equivalent of Formula (II) relative to the compound of Formula (B). In certain embodiments, the reacting of compound of Formula (IV) with a compound of Formula (B) is performed using 2 molar equivalent of Formula (II) relative to the compound of Formula (B). In certain embodiments, the reacting of compound of Formula (IV) with a compound of Formula (B) is performed using 3 molar equivalent of Formula (II) relative to the compound of Formula (B). In certain embodiments, the reacting of compound of Formula (IV) with a compound of Formula (B) is performed using 4 molar equivalent of Formula (II) relative to the compound of Formula (B). In certain embodiments, the reacting of compound of Formula (IV) with a compound of Formula (B) is performed using 5 molar equivalent of Formula (II) relative to the compound of Formula (B). In certain embodiments, the reacting of compound of Formula (IV) with a compound of Formula (B) is performed using 6 molar equivalent of Formula (II) relative to the compound of Formula (B). In certain embodiments, the reacting of compound of Formula (IV) with a compound of Formula (B) is performed using 7 molar equivalent of Formula (II) relative to the compound of Formula (B). In certain embodiments, the reacting of compound of Formula (IV) with a compound of Formula (B) is performed using 8 molar equivalent of Formula (II) relative to the compound of Formula (B). In certain embodiments, the reacting of compound of Formula (IV) with a compound of Formula (B) is performed using 9 molar equivalent of Formula (II) relative to the compound of Formula (B). In certain embodiments, the reacting of compound of Formula (IV) with a compound of Formula (B) is performed using 10 molar equivalent of Formula (II) relative to the compound of Formula (B).

In certain embodiments, the reacting of compound of Formula (IV) with a compound of Formula (B) results in formation of the compound of Formula (I) having an average ratio of molecular payload (R) to targeting agent (Q) of about 1 to about 4, about 1 to about 3, about 1 to about 2, about 2 to about 4, about 2 to about 3, or about 3 to about 4. In certain embodiments, the coupling of a targeting agent (Q) with a compound of Formula (II) results in formation of the compound of Formula (I) having an average ratio of molecular payload (R) to targeting agent (Q) of about 1, about 2, about 3, or about 4.

In certain embodiments, the reacting of a compound of Formula (IV) with a compound of Formula (B) takes place in solvent comprising dimethyl acetamide or isopropyl alcohol.

In certain embodiments, the method further comprises coupling a targeting agent (Q) with a compound of Formula (III):

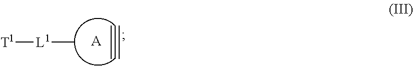

or a salt thereof, to provide a compound of Formula (B), wherein:
T¹ is

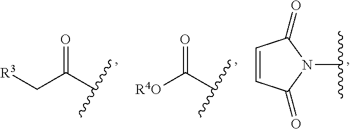

S═C═N—, or

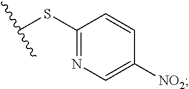 [SMILES: *Sc1ccc([N+](=O)[O-])cn1]

R³ is a leaving group; and
R⁴ is hydrogen; substituted or unsubstituted alkyl; substituted or unsubstituted heterocyclyl; substituted or unsubstituted aryl; substituted or unsubstituted heteroaryl; or an oxygen protecting group.

In certain embodiments, the method further comprises reacting a compound of Formula (V):

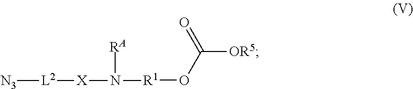

or a salt thereof, with a compound of Formula (VI):

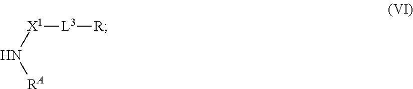

or a salt thereof, to provide a compound of Formula (IV), wherein R⁵ is substituted or unsubstituted aryl, or substituted or unsubstituted heteroaryl.

In certain embodiments, the method further comprises reacting a compound of Formula (VII):

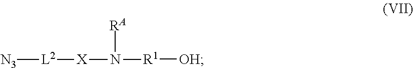

or a salt thereof, with a compound of Formula (VIII):

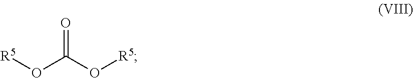

or a salt thereof, to provide a compound of Formula (V), wherein R⁵ is substituted or unsubstituted aryl, or substituted or unsubstituted heteroaryl

In certain embodiments, the method further comprises reacting a compound of Formula (IX):

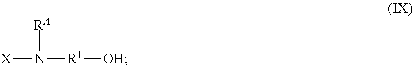

or a salt thereof, with a compound of Formula (X):

N₃-L²-LG (X);

or a salt thereof, to provide a compound of Formula (VII), wherein LG is a leaving group.

c. Method 3

In another aspect, disclosed is a method of preparing a compound of Formula (I):

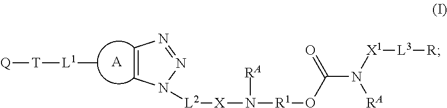

or a salt thereof, the method comprising
reacting a targeting agent (Q); a compound of Formula (IV):

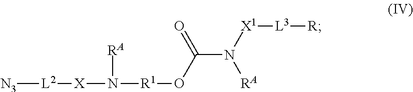

or a salt thereof; and a compound of Formula (III):

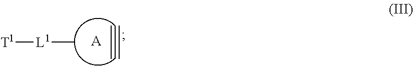

or a salt thereof; to provide a compound of Formula (I), or a salt thereof, wherein: T, L¹, A, L², X, R^{A}, R¹, X¹, L³, and R are as defined herein;
T¹ is

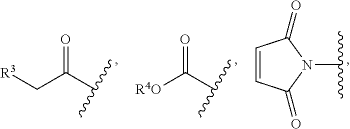

S═C═N—, or

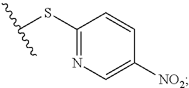 [SMILES: *Sc1ccc([N+](=O)[O-])cn1]

R³ is a leaving group;
R⁴ is hydrogen; substituted or unsubstituted alkyl; substituted or unsubstituted heterocyclyl; substituted or unsubstituted aryl; substituted or unsubstituted heteroaryl; or an oxygen protecting group; and
A¹ is substituted or unsubstituted carbocycle or substituted or unsubstituted heterocycle, or A¹ is absent and L¹ is bonded directly to one terminus of the alkyne.

In certain embodiments, the method further comprises reacting a compound of Formula (V):

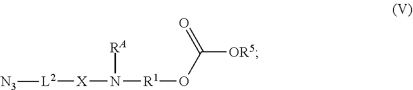

or a salt thereof, with a compound of Formula (VI):

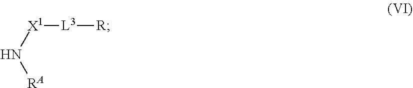

or a salt thereof, to provide a compound of Formula (IV), wherein:
R⁵ is substituted or unsubstituted aryl, or substituted or unsubstituted heteroaryl

In certain embodiments, the method further comprises reacting a compound of Formula (VII):

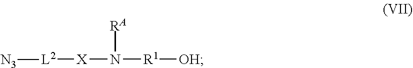

or a salt thereof, with a compound of Formula (VI):

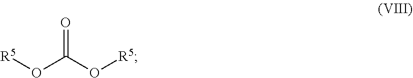

or a salt thereof, to provide a compound of Formula (V), wherein:
R⁵ is substituted or unsubstituted aryl, or substituted or unsubstituted heteroaryl.

In certain embodiments, the method further comprises comprising reacting a compound of Formula (IX):

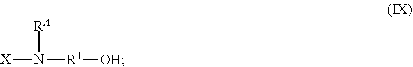

or a salt thereof, with a compound of Formula (X):

N₃-L²-LG (X);

or a salt thereof, to provide a compound of Formula (VII), wherein LG is a leaving group.

### Group T¹

As described herein, T¹ is:

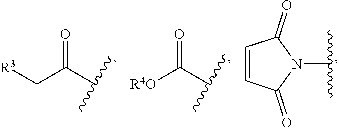

### S═C═N—, or

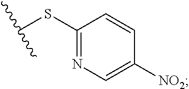

wherein:
R³ is a leaving group; and
R⁴ is hydrogen; substituted or unsubstituted alkyl; substituted or unsubstituted heterocyclyl; substituted or unsubstituted aryl; substituted or unsubstituted heteroaryl; or an oxygen protecting group.

In certain embodiments, R³ is halogen, tosylate, mesylate, or triflate.

In certain embodiments, R⁴ is substituted or unsubstituted alkyl; substituted or unsubstituted heterocyclyl; substituted or unsubstituted aryl; substituted or unsubstituted heteroaryl; or an oxygen protecting group. In certain embodiments, R⁴ is substituted or unsubstituted heterocyclyl; substituted or unsubstituted aryl; substituted or unsubstituted heteroaryl; or an oxygen protecting group. In certain embodiments, R⁴ is substituted or unsubstituted heterocyclyl; substituted or unsubstituted aryl; or substituted or unsubstituted heteroaryl. In certain embodiments, R⁴ is substituted or unsubstituted heterocyclyl; or substituted or unsubstituted aryl. In certain embodiments, R⁴ is substituted or unsubstituted heterocyclyl. In certain embodiments, R⁴ is substituted heterocyclyl. In certain embodiments, R⁴ is succinimide. In certain embodiments, R⁴ is sulfosuccinimide. In certain embodiments, R⁴ is 3-sulfosuccinimide. In certain embodiments, R⁴ is substituted or unsubstituted aryl. In certain embodiments, R⁴ is substituted aryl. In certain embodiments, R⁴ is substituted phenyl. In certain embodiments, R⁴ is pentafluorophenyl. In certain embodiments, R⁴ is tetrafluorophenyl. In certain embodiments, R⁴ is 4-nitrophenyl.

In certain embodiments, T¹ is:

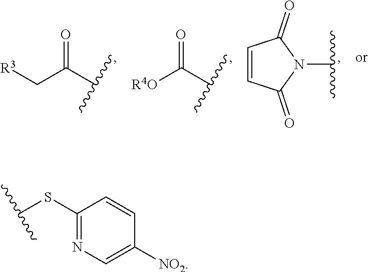

In certain embodiments, T¹ is:

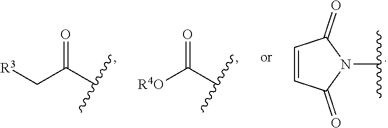

In certain embodiments, T¹ is:

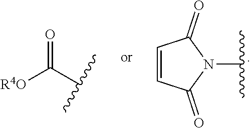

In certain embodiments, T¹ is:

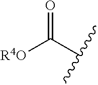

In certain embodiments, T¹ is:

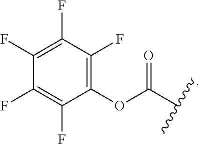 [SMILES: *C(=O)Oc1c(F)c(F)c(F)c(F)c1F]

In certain embodiments, T¹ is:

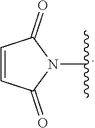 [SMILES: *N1C(=O)C=CC1=O]

### Group A¹

As described herein, A¹ is substituted or unsubstituted carbocycle or substituted or unsubstituted heterocycle, or A¹ is absent and L¹ is bonded directly to one terminus of the alkyne (i.e., L¹-A¹ is

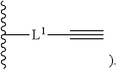

In certain embodiments, A¹ is substituted or unsubstituted carbocycle, or substituted or unsubstituted heterocycle. In certain embodiments, A¹ is absent and L¹ is bonded directly one terminus of the alkyne (i.e., L¹-A¹ is

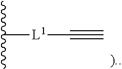

In certain embodiments, A¹ is substituted or unsubstituted heterocycle. In certain embodiments, A¹ is substituted or unsubstituted 5-8 membered heterocycle. In certain embodiments, A¹ is substituted or unsubstituted 6-8 membered heterocycle. In certain embodiments, A¹ is substituted or unsubstituted 7-8 membered heterocycle. In certain embodiments, A¹ is a substituted or unsubstituted 8-membered heterocycle. In certain embodiments, A¹ is a substituted 8-membered heterocycle. In certain embodiments, A¹ is a substituted or unsubstituted hexahydroazocine. In certain embodiments, A¹ is a substituted or unsubstituted tetrahydroazocine. In certain embodiments, A¹ is a substituted or unsubstituted dihydroazocine.

In certain embodiments, A¹ is:

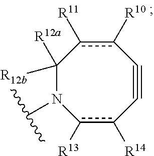

wherein:
[symbol] is a single or double bond;
R¹⁰ is hydrogen, halogen, alkoxy, substituted or unsubstituted alkyl, or substituted or unsubstituted aryl;
R¹¹ is hydrogen, halogen, alkoxy, substituted or unsubstituted alkyl, or substituted or unsubstituted aryl; or R¹⁰ and R¹¹ together with the atoms to which they are attached form a substituted or unsubstituted aryl;
R^{12a} and R^{12b} are each hydrogen or together with the carbon to which they are attached form a carbonyl;
R¹³ is hydrogen, halogen, alkoxy, substituted or unsubstituted alkyl, or substituted or unsubstituted aryl; and
R¹⁴ is hydrogen, halogen, alkoxy, substituted or unsubstituted alkyl, or substituted or unsubstituted aryl; or R¹³ and R¹⁴ together with the atoms to which they are attached form a substituted or unsubstituted aryl.

In certain embodiments A¹ is:

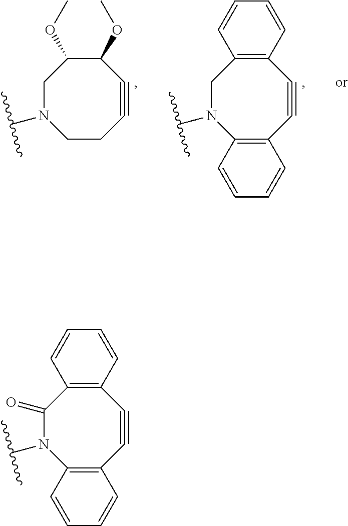 [SMILES: *N1CCC#C[C@H](OC)[C@@H](OC)C1 ; *N1Cc2ccccc2C#Cc2ccccc21 ; *N1C(=O)c2ccccc2C#Cc2ccccc21]

In certain embodiments, A¹ is:

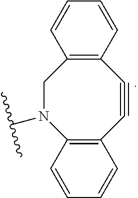 [SMILES: *N1Cc2ccccc2C#Cc2ccccc21]

In certain embodiments, A¹ is substituted or unsubstituted carbocycle. In certain embodiments, A¹ is substituted or unsubstituted C₅₋₁₀ carbocycle. In certain embodiments, A¹ is substituted or unsubstituted C₈₋₁₀ carbocycle. In certain embodiments, A¹ is substituted or unsubstituted C₈₋₉ carbocycle. In certain embodiments, A¹ is a substituted or unsubstituted C₈ carbocycle. In certain embodiments, A¹ is a substituted or unsubstituted cyclooctene.

In certain embodiments, A¹ is:

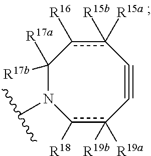

wherein:
[symbol] is a single or double bond;
R^{15a} and R^{15b} are each independently hydrogen, halogen, alkoxy, substituted or unsubstituted alkyl, or substituted or unsubstituted aryl;
R¹⁶ is hydrogen, halogen, alkoxy, substituted or unsubstituted alkyl, or substituted or unsubstituted aryl; or one of R^{15a}/R^{15b} and R¹⁶ together with the atoms to which they are attached form a substituted or unsubstituted aryl;
R^{17a} and R^{17b} are each hydrogen or together with the carbon to which they are attached form a carbonyl;
R¹⁸ is hydrogen, halogen, alkoxy, substituted or unsubstituted alkyl, or substituted or unsubstituted aryl; and
R^{19a} and R^{19b} are each independently hydrogen, halogen, alkoxy, substituted or unsubstituted alkyl, or substituted or unsubstituted aryl.

In certain embodiments, A¹ is:

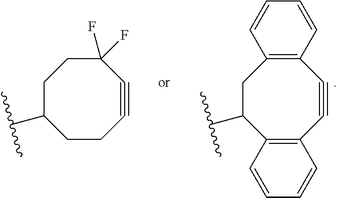 [SMILES: *C1CCC#CC(F)(F)CC1 ; *C1Cc2ccccc2C#Cc2ccccc21]

In certain embodiments, A¹ is:

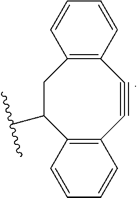 [SMILES: *C1Cc2ccccc2C#Cc2ccccc21]

In certain embodiments, A¹ is:

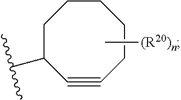

wherein:
each R²⁰ is independently halogen, alkoxy, substituted or unsubstituted alkyl, or substituted or unsubstituted aryl; or two occurrences of R²⁰ together with the atoms to which they are attached form a substituted or unsubstituted aryl or carbocyclic ring; and
n is 0-8.

In certain embodiments, A¹ is:

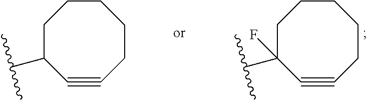 [SMILES: *C1C#CCCCCC1 ; *C1(F)C#CCCCCC1]

In certain embodiments, A¹ is a substituted or unsubstituted C₉ carbocycle. In certain embodiments, A¹ is a substituted or unsubstituted bicyclic fused C₉ carbocycle. In certain embodiments, A¹ is a substituted or unsubstituted bicyclic fused C₉ cycloalkyl or cycloalkenyl. In certain embodiments, A¹ is a substituted or unsubstituted bicyclo[6.1.0]non-4-enyl. In certain embodiments, A¹ is an unsubstituted bicyclo[6.1.0]non-4-enyl.

In certain embodiments, A¹ is:

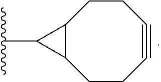 [SMILES: *C1C2CCC#CCCC12]

In certain embodiments, A is:

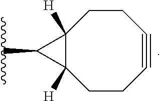 [SMILES: *[C@@H]1[C@H]2CCC#CCC[C@@H]12]

In certain embodiments, A¹ is:

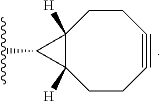 [SMILES: *[C@H]1[C@H]2CCC#CCC[C@@H]12]

### Group R⁵

As described herein, R⁵ is substituted or unsubstituted aryl, or substituted or unsubstituted heteroaryl. In certain embodiments, R⁵ is substituted or unsubstituted aryl. R⁵ is substituted or unsubstituted phenyl. In certain embodiments, R⁵ is substituted phenyl. In certain embodiments, R⁵ is p-nitrophenyl.

In certain embodiments of methods 1, 2, and 3, the compound of Formula (I) is any of Formula (I-a), (I-b), (I-c), (I-d), (I-e), (I-f), (I-g), (I-h), (I-i), (I-j), (I-j-1), (I-j-2), (I-j-3), (I-k), (I-k-1), (I-k-2), (I-m), (I-m-1), and (I-m-2).

In certain embodiments of methods 1, 2, and 3, the compound of Formula (II) is of Formula (II-a):

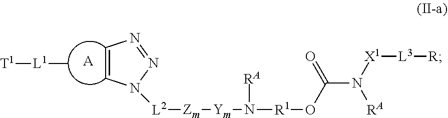

or a salt thereof, wherein T¹, L¹, A, L², Z, Y, m, R^{A}, R¹, X¹, L³, and R are as defined herein.

In certain embodiments, the compound of Formula (II) is a compound of Formula (II-b):

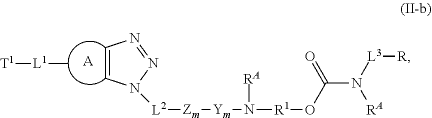

or a pharmaceutically acceptable salt, tautomer, stereoisomer, or isotopically enriched derivative thereof, wherein T¹, L¹, A, L², Z, Y, m, R^{A}, R¹, L³, and R are as defined herein.

In certain embodiments, the compound of Formula (II) is a compound of Formula (II-c):

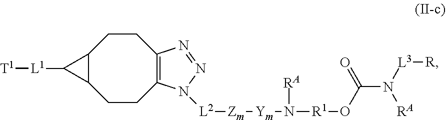

or a pharmaceutically acceptable salt, tautomer, stereoisomer, or isotopically enriched derivative thereof, wherein T¹, L¹, L², Z, Y, m, R^{A}, R¹, L³, and R are as defined herein.

In certain embodiments, the compound of Formula (II) is a compound of Formula (II-d):

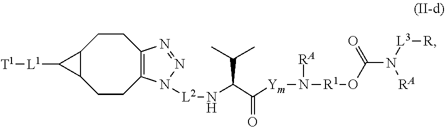

or a pharmaceutically acceptable salt, tautomer, stereoisomer, or isotopically enriched derivative thereof, wherein T¹, L¹, L², Y, m, R^{A}, R¹, L³, and R are as defined herein.

In certain embodiments, the compound of Formula (II) is a compound of Formula (II-e):

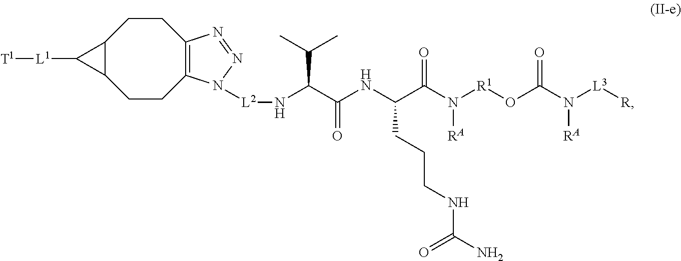

or a pharmaceutically acceptable salt, tautomer, stereoisomer, or isotopically enriched derivative thereof, wherein T¹, L¹, L², R^{A}, R¹, L³, and R are as defined herein.

In certain embodiments, the compound of Formula (II) is a compound of Formula (II-f):

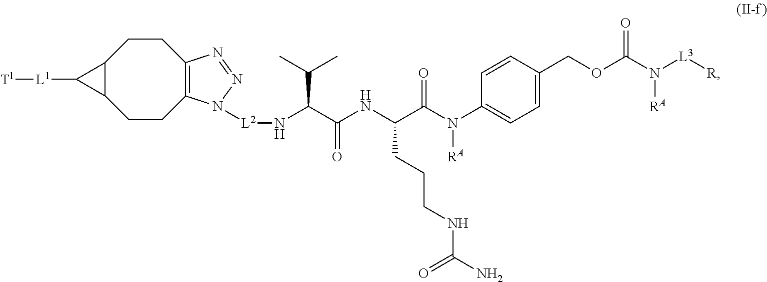

or a pharmaceutically acceptable salt, tautomer, stereoisomer, or isotopically enriched derivative thereof, wherein T¹, L¹, L², R^{A}, L³, and R are as defined herein.

In certain embodiments, the compound of Formula (II) is a compound of Formula (II-g):

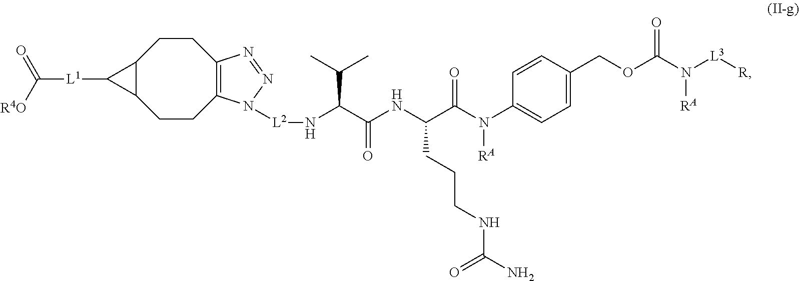

or a pharmaceutically acceptable salt, tautomer, stereoisomer, or isotopically enriched derivative thereof, wherein R⁴, L¹, L², R^{A}, L³, and R are as defined herein.

In certain embodiments, the compound of Formula (II) is a compound of Formula (II-h):

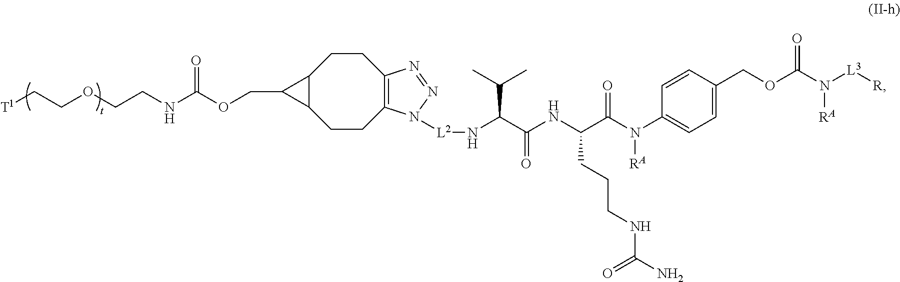

or a pharmaceutically acceptable salt, tautomer, stereoisomer, or isotopically enriched derivative thereof, wherein T¹, t, L², R^{A}, L³, and R are as defined herein.

In certain embodiments, the compound of Formula (II) is a compound of Formula (II-i):

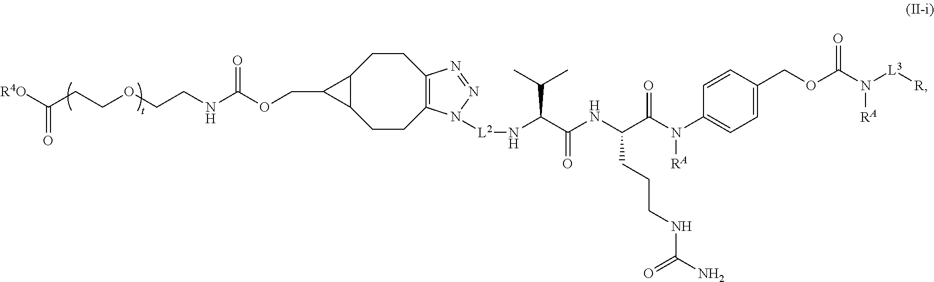

or a pharmaceutically acceptable salt, tautomer, stereoisomer, or isotopically enriched derivative thereof, wherein R⁴, t, L², R^{A}, L³, and R are as defined herein.

In certain embodiments, the compound of Formula (II) is a compound of Formula (II-j):

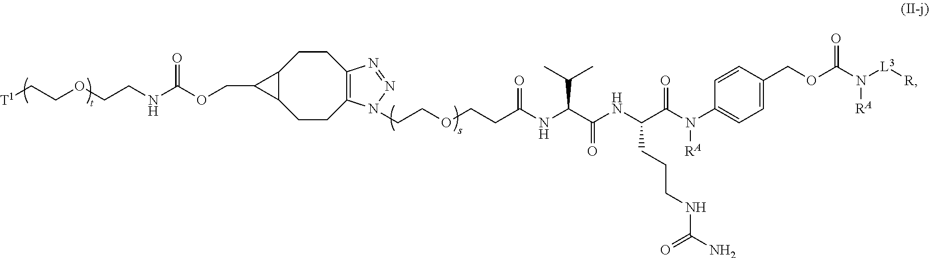

or a pharmaceutically acceptable salt, tautomer, stereoisomer, or isotopically enriched derivative thereof, wherein T¹, t, s, R^{A}, L³, and R are as defined herein.

In certain embodiments, the compound of Formula (II) is a compound of Formula (II-j-1):

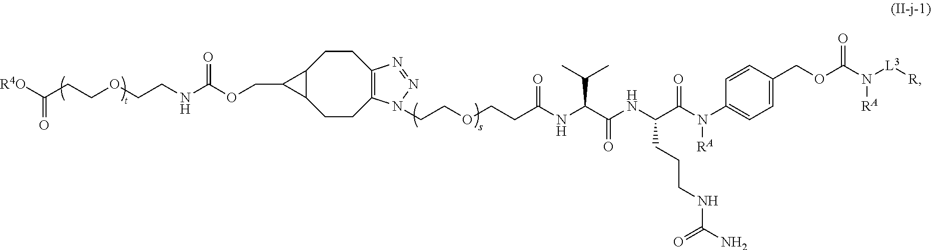

or a pharmaceutically acceptable salt, tautomer, stereoisomer, or isotopically enriched derivative thereof, wherein R⁴, t, s, R^{A}, L³, and R are as defined herein.

In certain embodiments, the compound of Formula (II) is a compound of Formula (II-j-2):

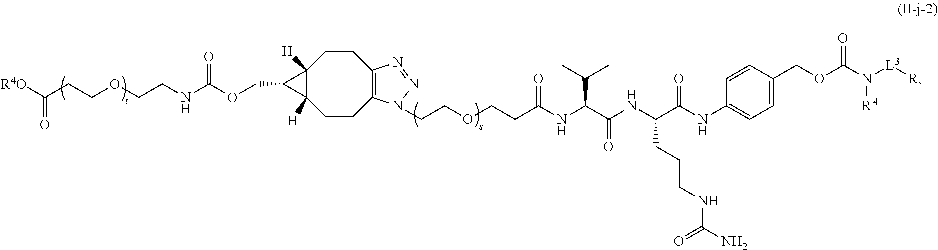

or a pharmaceutically acceptable salt, tautomer, stereoisomer, or isotopically enriched derivative thereof, wherein R⁴, t, s, L³, and R are as defined herein.

In certain embodiments, the compound of Formula (II) is a compound of Formula (II-j-3):

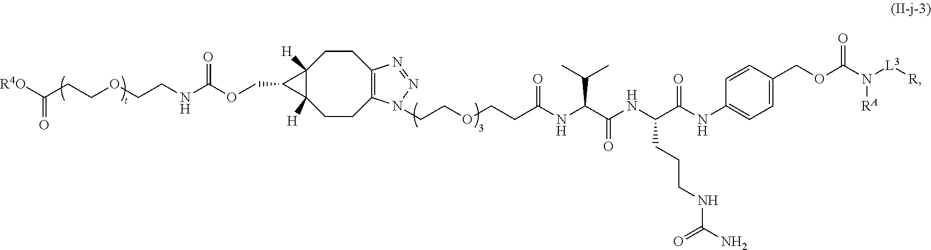

or a pharmaceutically acceptable salt, tautomer, stereoisomer, or isotopically enriched derivative thereof, wherein R⁴, t, s, L³, and R are as defined herein.

In certain embodiments, the compound of Formula (II) is a compound of Formula (II-k):

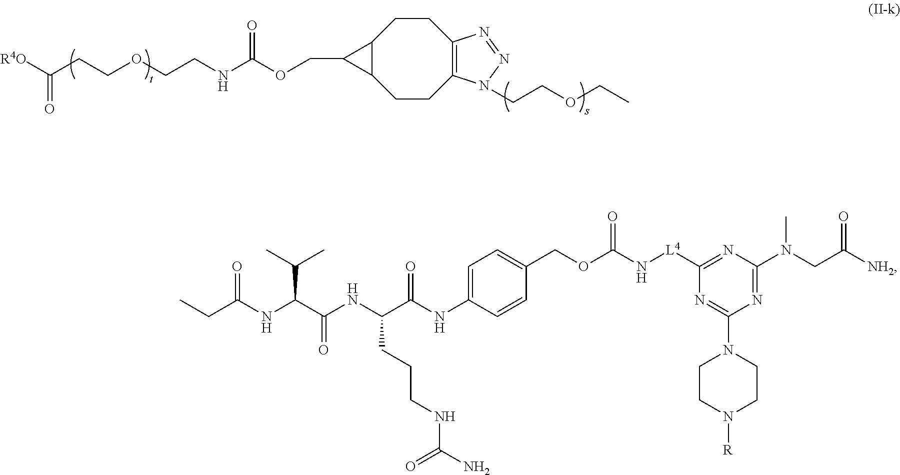

or a pharmaceutically acceptable salt, tautomer, stereoisomer, or isotopically enriched derivative thereof, wherein R⁴, L⁴, R, t, and s are as defined herein.

In certain embodiments, the compound of Formula (II) is a compound of Formula (II-k-1):

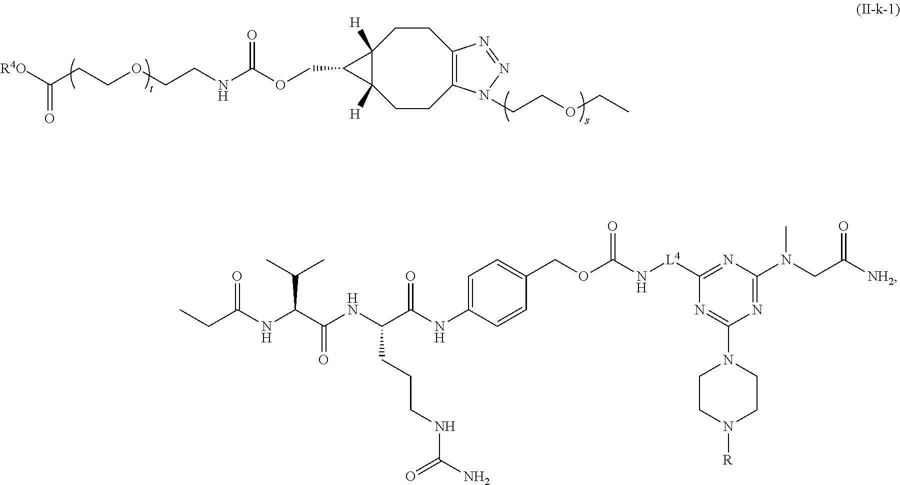

or a pharmaceutically acceptable salt, tautomer, stereoisomer, or isotopically enriched derivative thereof, wherein R⁴, L⁴, R, t, and s are as defined herein.

In certain embodiments, the compound of Formula (II) is a compound of Formula (II-k-2):

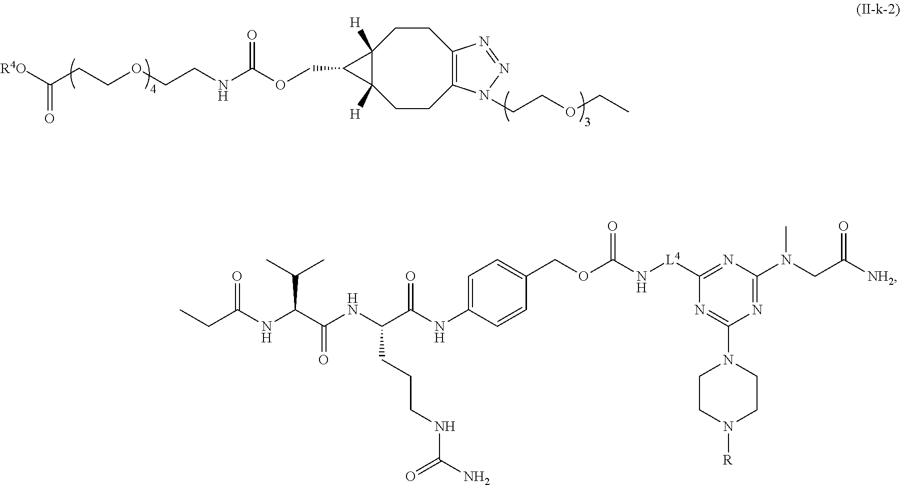

or a pharmaceutically acceptable salt, tautomer, stereoisomer, or isotopically enriched derivative thereof, wherein R⁴, L⁴, and R are as defined herein. In certain embodiments of Formula (II-k-2), R⁴ is pentafluorophenyl.

In certain embodiments, the compound of Formula (II) is a compound of Formula (II-m):

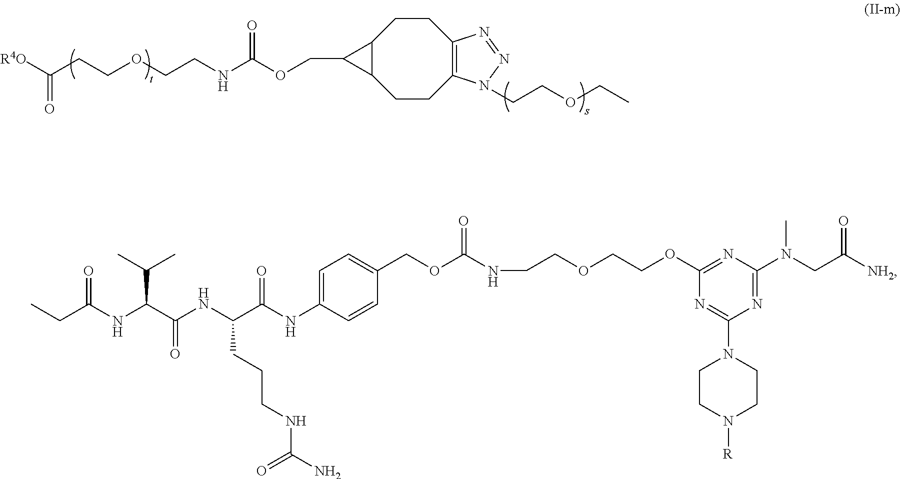

or a pharmaceutically acceptable salt, tautomer, stereoisomer, or isotopically enriched derivative thereof, wherein R⁴, t, s, and R are as defined herein.

In certain embodiments, the compound of Formula (II) is a compound of Formula

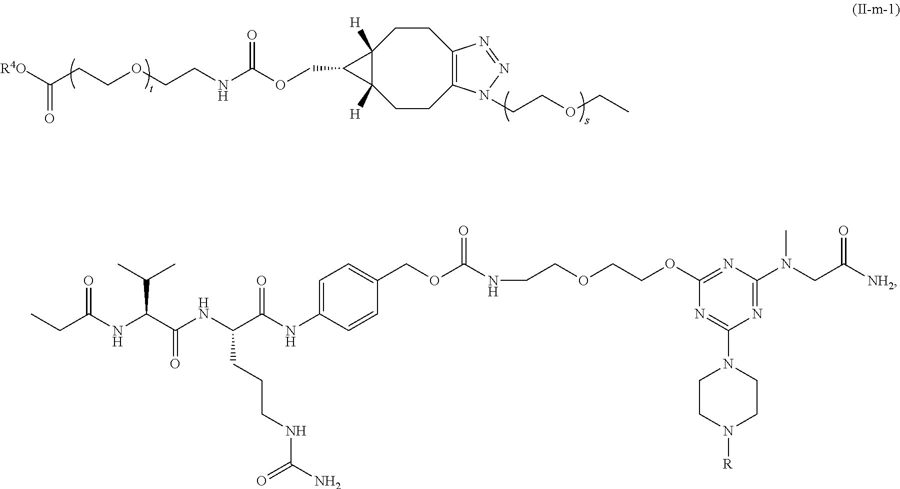

or a pharmaceutically acceptable salt, tautomer, stereoisomer, or isotopically enriched derivative thereof, wherein R⁴, t, s, and R are as defined herein.

In certain embodiments, the compound of Formula (II) is a compound of Formula (II-m-2):

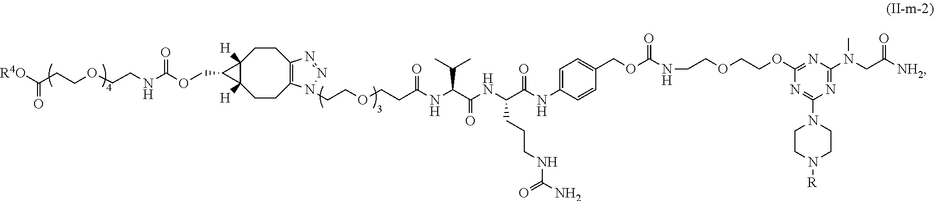

or a pharmaceutically acceptable salt, tautomer, stereoisomer, or isotopically enriched derivative thereof, wherein R⁴ and R are as defined herein. In certain embodiments of Formula (II-m-2), R⁴ is pentafluorophenyl.

In certain embodiments of methods 1, 2, and 3, the compound of Formula (III) is of Formula (III-a):

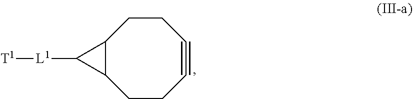

or a pharmaceutically acceptable salt, tautomer, stereoisomer, or isotopically enriched derivative thereof, wherein T¹ and L¹ are as defined herein.

In certain embodiments, the compound of Formula (III) is of Formula (III-b):

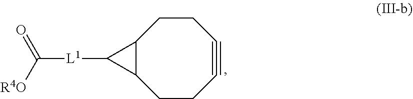

or a pharmaceutically acceptable salt, tautomer, stereoisomer, or isotopically enriched derivative thereof, wherein R⁴ and L¹ are as defined herein.

In certain embodiments, the compound of Formula (III) is of Formula (III-c):

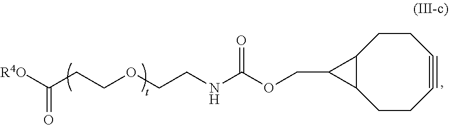

or a pharmaceutically acceptable salt, tautomer, stereoisomer, or isotopically enriched derivative thereof, wherein R⁴ and t are as defined herein.

In certain embodiments, the compound of Formula (III) is of Formula (III-d):

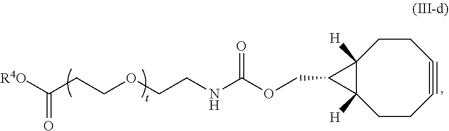

or a pharmaceutically acceptable salt, tautomer, stereoisomer, or isotopically enriched derivative thereof, wherein R⁴ and t are as defined herein.

In certain embodiments, the compound of Formula (III) is of Formula (III-e):

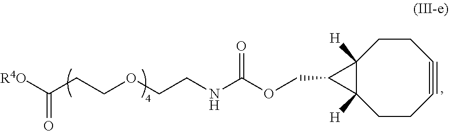

or a pharmaceutically acceptable salt, tautomer, stereoisomer, or isotopically enriched derivative thereof, wherein R⁴ is as defined herein.

In certain embodiments, the compound of Formula (III) is of Formula (III-f):

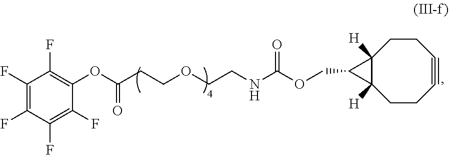

or a pharmaceutically acceptable salt, tautomer, stereoisomer, or isotopically enriched derivative thereof.

In certain embodiments of methods 1, 2, and 3, the compound of Formula (B) is of Formula (B-a):

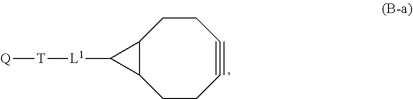

or a pharmaceutically acceptable salt, tautomer, stereoisomer, or isotopically enriched derivative thereof, wherein Q, T and L¹ are as defined herein.

In certain embodiments, the compound of Formula (B) is of Formula (B-b):

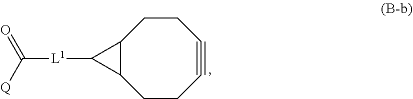

or a pharmaceutically acceptable salt, tautomer, stereoisomer, or isotopically enriched derivative thereof, wherein Q and L¹ are as defined herein.

In certain embodiments, the compound of Formula (B) is of Formula (B-c):

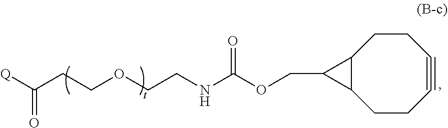

or a pharmaceutically acceptable salt, tautomer, stereoisomer, or isotopically enriched derivative thereof, wherein Q and t are as defined herein.

In certain embodiments, the compound of Formula (B) is of Formula (B-d):

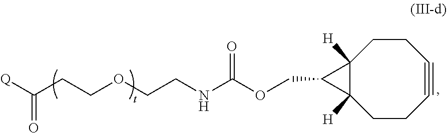

or a pharmaceutically acceptable salt, tautomer, stereoisomer, or isotopically enriched derivative thereof, wherein Q and t are as defined herein.

In certain embodiments, the compound of Formula (B) is of Formula (B-e):

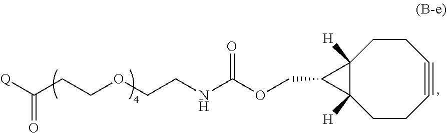

or a pharmaceutically acceptable salt, tautomer, stereoisomer, or isotopically enriched derivative thereof, wherein Q is as defined herein. In certain embodiments of the compound of Formula (B-e), Q is an antibody. In certain embodiments of the compound of Formula (B-e), Q is an anti-TfR antibody.

In certain embodiments of methods 1, 2, and 3, the compound of Formula (IV) is of Formula (IV-a):

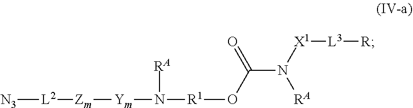

or a salt thereof, wherein L², Z, Y, m, R^{A}, R¹, X¹, L³, and R are as defined herein.

In certain embodiments, the compound of Formula (IV) is a compound of Formula (IV-b):

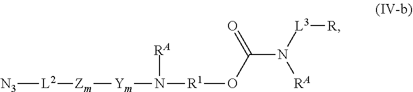

or a pharmaceutically acceptable salt, tautomer, stereoisomer, or isotopically enriched derivative thereof, wherein L², Z, Y, m, R^{A}, R¹, L³, and R are as defined herein.

In certain embodiments, the compound of Formula (IV) is a compound of Formula (IV-c):

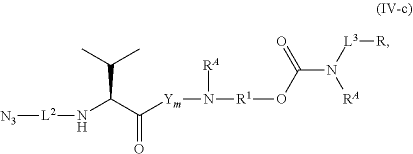

or a pharmaceutically acceptable salt, tautomer, stereoisomer, or isotopically enriched derivative thereof, wherein L², Y, m, R^{A}, R¹, L³, and R are as defined herein.

In certain embodiments, the compound of Formula (IV) is a compound of Formula (IV-d):

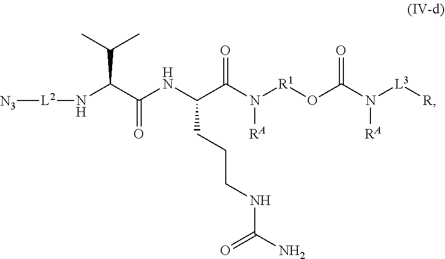

or a pharmaceutically acceptable salt, tautomer, stereoisomer, or isotopically enriched derivative thereof, wherein L², R^{A}, R¹, L³, and R are as defined herein.

In certain embodiments, the compound of Formula (IV) is a compound of Formula (IV-e):

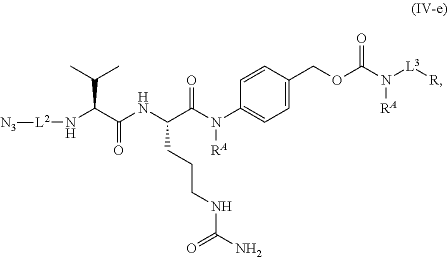

or a pharmaceutically acceptable salt, tautomer, stereoisomer, or isotopically enriched derivative thereof, wherein L², R^{A}, L³, and R are as defined herein.

In certain embodiments, the compound of Formula (IV) is a compound of Formula (IV-f):

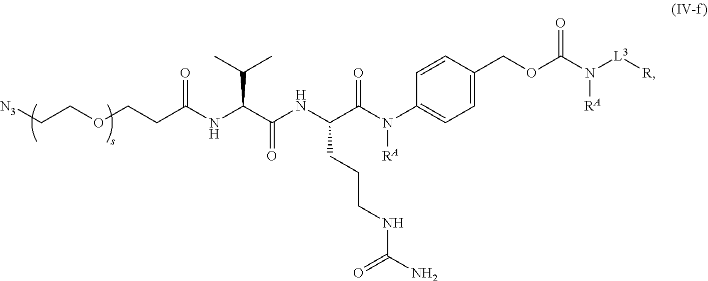

or a pharmaceutically acceptable salt, tautomer, stereoisomer, or isotopically enriched derivative thereof, wherein s, R^{A}, L³, and R are as defined herein.

In certain embodiments, the compound of Formula (IV) is a compound of Formula (IV-g):

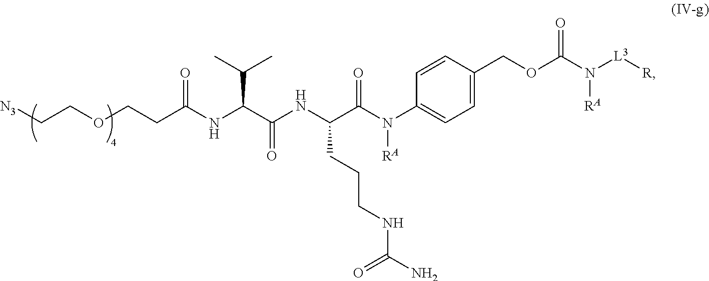

or a pharmaceutically acceptable salt, tautomer, stereoisomer, or isotopically enriched derivative thereof, wherein R^{A}, L³, and R are as defined herein.

In certain embodiments, the compound of Formula (IV) is a compound of Formula (IV-h):

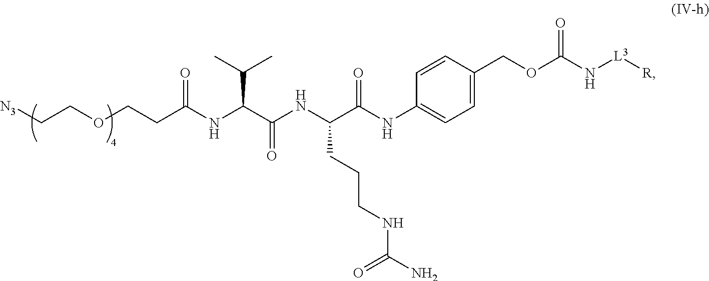

or a pharmaceutically acceptable salt, tautomer, stereoisomer, or isotopically enriched derivative thereof, wherein L³ and R are as defined herein.

In certain embodiments, the compound of Formula (IV) is a compound of Formula (IV-i):

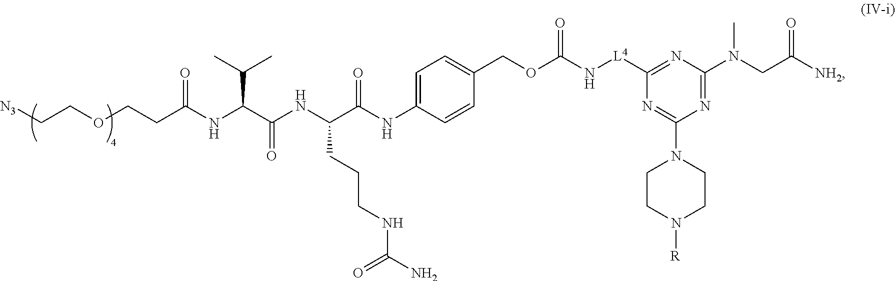

or a pharmaceutically acceptable salt, tautomer, stereoisomer, or isotopically enriched derivative thereof, wherein L⁴ and R are as defined herein.

In certain embodiments, the compound of Formula (IV) is a compound of Formula (IV-k):

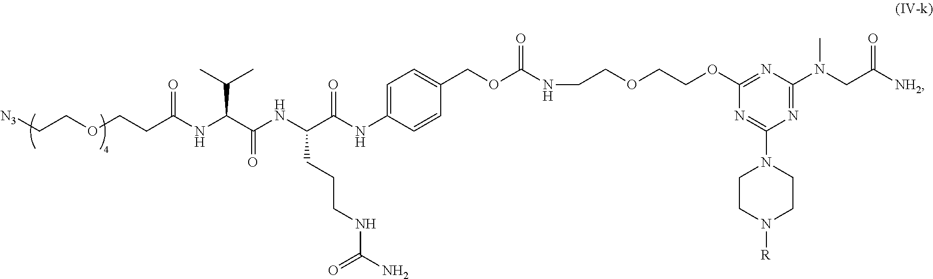

or a pharmaceutically acceptable salt, tautomer, stereoisomer, or isotopically enriched derivative thereof, wherein R is as defined herein.

In certain embodiments of methods 1, 2, and 3, the compound of Formula (V) is of Formula (V-a):

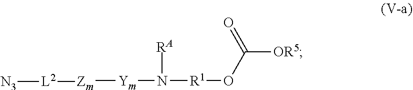

or a salt thereof, wherein L², Z, Y, m, R^{A}, R¹, and R⁵ are as defined herein.

In certain embodiments, the compound of Formula (V) is a compound of Formula (V-b):

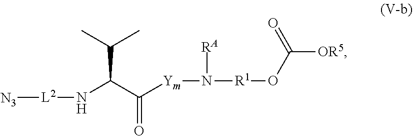

or a pharmaceutically acceptable salt, tautomer, stereoisomer, or isotopically enriched derivative thereof, wherein L², Y, m, R^{A}, R¹, and R⁵ are as defined herein.

In certain embodiments, the compound of Formula (V) is a compound of Formula (V-c):

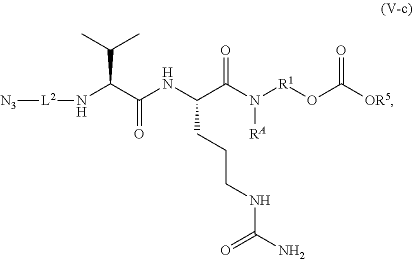

or a pharmaceutically acceptable salt, tautomer, stereoisomer, or isotopically enriched derivative thereof, wherein L², R^{A}, R¹, and R⁵ are as defined herein.

In certain embodiments, the compound of Formula (V) is a compound of Formula (V-d):

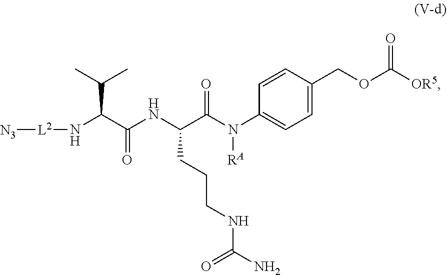

or a pharmaceutically acceptable salt, tautomer, stereoisomer, or isotopically enriched derivative thereof, wherein L², R^{A}, and R⁵ are as defined herein.

In certain embodiments, the compound of Formula (V) is a compound of Formula (V-e):

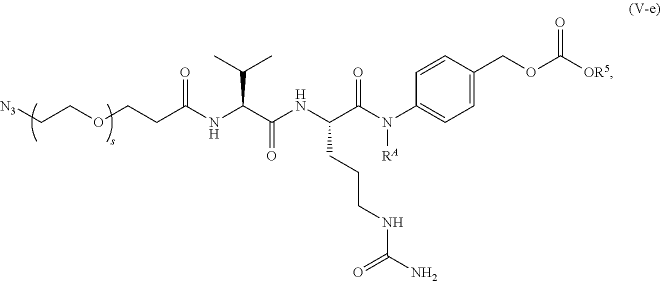

or a pharmaceutically acceptable salt, tautomer, stereoisomer, or isotopically enriched derivative thereof, wherein s, R^{A}, and R⁵ are as defined herein.

In certain embodiments, the compound of Formula (V) is a compound of Formula (V-f):

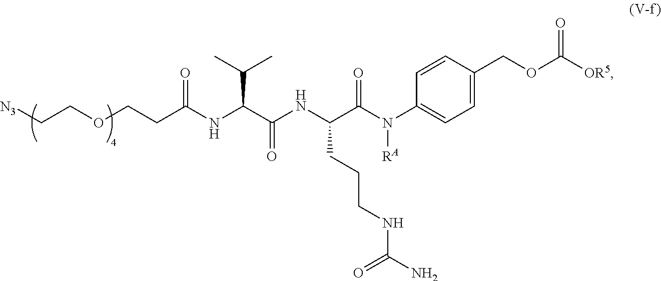

or a pharmaceutically acceptable salt, tautomer, stereoisomer, or isotopically enriched derivative thereof, wherein R^{A} and R⁵ are as defined herein.

In certain embodiments, the compound of Formula (V) is a compound of Formula (V-g):

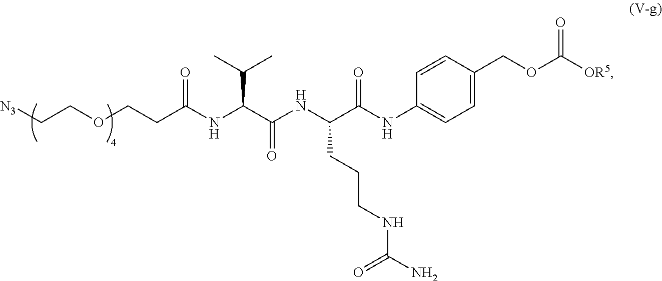

or a pharmaceutically acceptable salt, tautomer, stereoisomer, or isotopically enriched derivative thereof, wherein R⁵ is as defined herein.

In certain embodiments, the compound of Formula (V) is a compound of Formula (V-h):

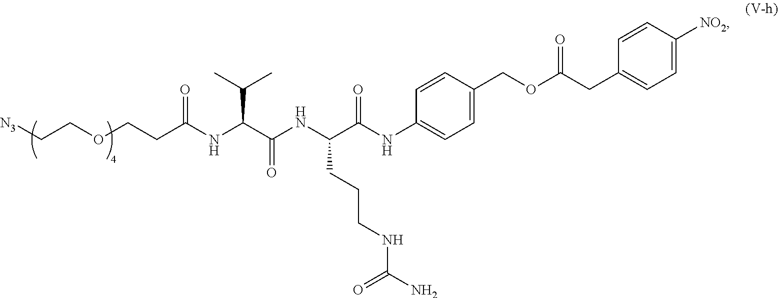

or a pharmaceutically acceptable salt, tautomer, stereoisomer, or isotopically enriched derivative thereof.

In certain embodiments of methods 1, 2, and 3, the compound of Formula (VI) is of Formula (VI-a):

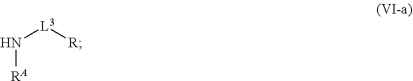

or a salt thereof, wherein R^{A}, L³, and R are as defined herein.

In certain embodiments, the compound of Formula (VI) is a compound of Formula (VI-b):

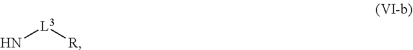

or a pharmaceutically acceptable salt, tautomer, stereoisomer, or isotopically enriched derivative thereof, wherein L³ and R are as defined herein.

In certain embodiments, the compound of Formula (VI) is a compound of Formula (VI-c):

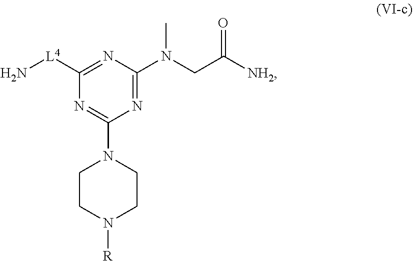

or a pharmaceutically acceptable salt, tautomer, stereoisomer, or isotopically enriched derivative thereof, wherein R is as defined herein.

In certain embodiments, the compound of Formula (VI) is a compound of Formula (VI-e):

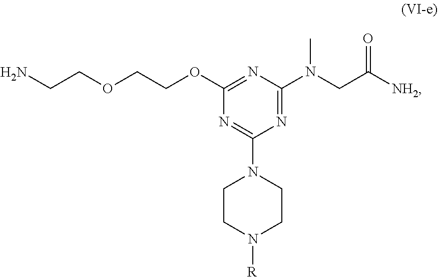

or a pharmaceutically acceptable salt, tautomer, stereoisomer, or isotopically enriched derivative thereof, wherein R is as defined herein.

In certain embodiments of Formula (VI), R is an oligonucleotide. In certain embodiments of Formula (VI), R is an antisense oligonucleotide. In certain embodiments of Formula (VI), R is a phosphorodiamidate morpholino oligomer (PMO). In certain embodiments of Formula (VI), R is a gapmer. In certain embodiments of Formula (VI), R is an siRNA.

In certain embodiments of methods 1, 2, and 3, the compound of Formula (VII) is of Formula (VII-a):

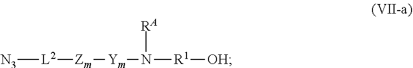

or a salt thereof, wherein L², Z, Y, m, R^{A}, and R¹ are as defined herein.

In certain embodiments, the compound of Formula (VII) is a compound of Formula (VII-b):

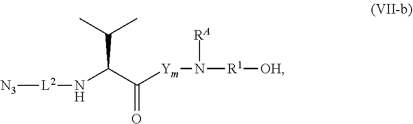

or a pharmaceutically acceptable salt, tautomer, stereoisomer, or isotopically enriched derivative thereof, wherein L², Y, m, R^{A}, and R¹ are as defined herein.

In certain embodiments, the compound of Formula (VII) is a compound of Formula (VII-c):

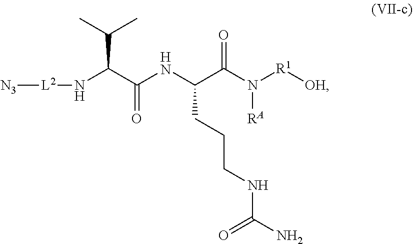

or a pharmaceutically acceptable salt, tautomer, stereoisomer, or isotopically enriched derivative thereof, wherein L², R^{A}, and R¹ are as defined herein.

In certain embodiments, the compound of Formula (VII) is a compound of Formula (VII-d):

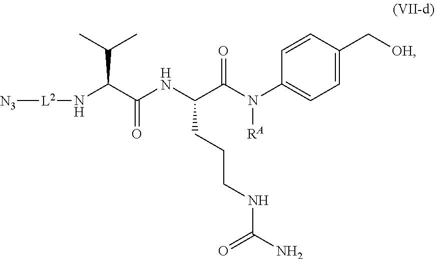

or a pharmaceutically acceptable salt, tautomer, stereoisomer, or isotopically enriched derivative thereof, wherein L² and R^{A} are as defined herein.

In certain embodiments, the compound of Formula (VII) is a compound of Formula (VII-e):

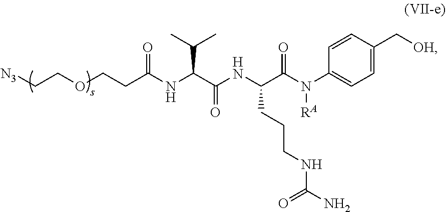

or a pharmaceutically acceptable salt, tautomer, stereoisomer, or isotopically enriched derivative thereof, wherein s and R^{A} are as defined herein.

In certain embodiments, the compound of Formula (VII) is a compound of Formula (VII-f):

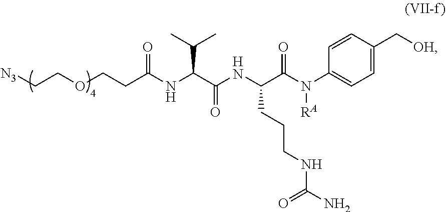

or a pharmaceutically acceptable salt, tautomer, stereoisomer, or isotopically enriched derivative thereof, wherein R^{A} is as defined herein.

In certain embodiments, the compound of Formula (VII) is a compound of Formula (VII-g):

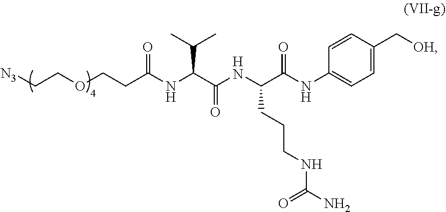

or a pharmaceutically acceptable salt, tautomer, stereoisomer, or isotopically enriched derivative thereof.

In certain embodiments of methods 1, 2, and 3, the compound of Formula (IX) is of Formula (IX-a):

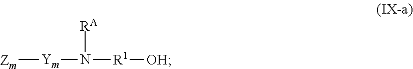

or a salt thereof, wherein Z, Y, m, R^{A}, and R¹ are as defined herein.

In certain embodiments, the compound of Formula (IX) is a compound of Formula (IX-b):

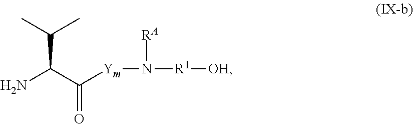

or a pharmaceutically acceptable salt, tautomer, stereoisomer, or isotopically enriched derivative thereof, wherein Y, m, R^{A}, and R¹ are as defined herein.

In certain embodiments, the compound of Formula (IX) is a compound of Formula (IX-c):

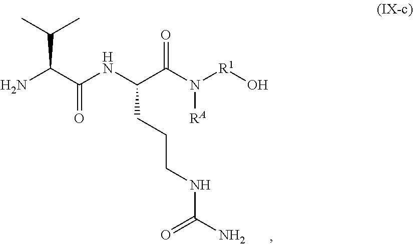

or a pharmaceutically acceptable salt, tautomer, stereoisomer, or isotopically enriched derivative thereof, wherein R^{A} and R¹ are as defined herein.

In certain embodiments, the compound of Formula (IX) is a compound of Formula (IX-d):

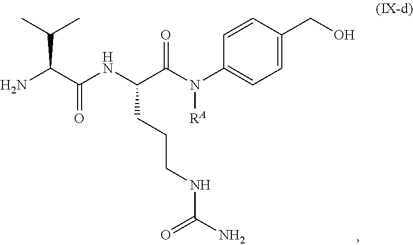

or a pharmaceutically acceptable salt, tautomer, stereoisomer, or isotopically enriched derivative thereof, wherein R^{A} is as defined herein.

In certain embodiments, the compound of Formula (IX) is a compound of Formula (IX-e):

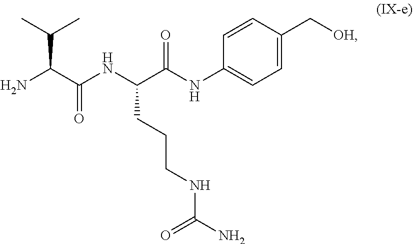 [SMILES: CC(C)[C@H](N)C(=O)N[C@@H](CCCNC(N)=O)C(=O)Nc1ccc(CO)cc1]

or a pharmaceutically acceptable salt, tautomer, stereoisomer, or isotopically enriched derivative thereof.

In certain embodiments of methods 1, 2, and 3, the compound of Formula (X) is of Formula (X-a):

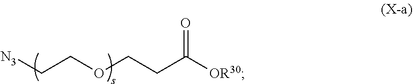

or a salt thereof, wherein s is as defined herein; and R³⁰ is a substituted or unsubstituted heterocycle.

In certain embodiments of methods 1, 2, and 3, the compound of Formula (X) is of Formula (X-b):

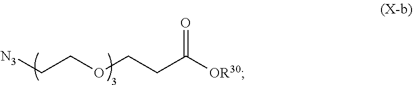

or a pharmaceutically acceptable salt, tautomer, stereoisomer, or isotopically enriched derivative thereof, wherein s is as defined herein; and R³⁰ is a substituted or unsubstituted heterocycle. In certain embodiments, R³⁰ is a substituted heterocycle. R³⁰ is a unsubstituted heterocycle.

In certain embodiments, the compound of Formula (X) is a compound of Formula (X-c):

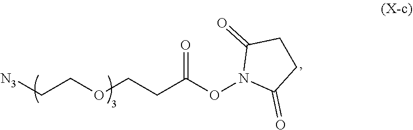

or a pharmaceutically acceptable salt, tautomer, stereoisomer, or isotopically enriched derivative thereof.

### IV. Methods of Purifying Certain Compounds

Also disclosed herein are methods of purifying compounds of Formula (II):

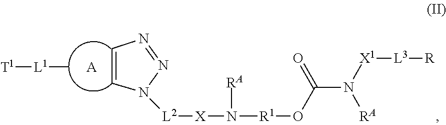

or a pharmaceutically acceptable salt, tautomer, stereoisomer, or isotopically enriched derivative thereof.

In certain embodiments, purifying the compound of Formula (II) comprises adding a volume of a solvent that is at least 2, 3, 4, 5, 6, 7, 8, 9, or 10 times the amount of the total volume of a reaction mixture comprising the compound of Formula (II); and isolating the compound of Formula (II). In certain embodiments, purifying the compound of Formula (II) comprises adding a volume of a solvent that is at least 8 times the amount of the total volume of a reaction mixture comprising the compound of Formula (II); and isolating the compound of Formula (II). In certain embodiments, purifying the compound of Formula (II) comprises adding a volume of a solvent that is at least 3 times the amount of a reaction mixture comprising the compound of Formula (II); and isolating the compound of Formula (II). In certain embodiments, purifying the compound of Formula (II) comprises adding a volume of a solvent that is about 2, 3, 4, 5, 6, 7, 8, 9, or 10 times the amount of the total volume of a reaction mixture comprising the compound of Formula (II); and isolating the compound of Formula (II). In certain embodiments, purifying the compound of Formula (II) comprises adding a volume of a solvent that is about 8 times the amount of the total volume of a reaction mixture comprising the compound of Formula (II); and isolating the compound of Formula (II). In certain embodiments, purifying the compound of Formula (II) comprises adding a volume of a solvent that is about 3 times the amount of the total volume of a reaction mixture comprising the compound of Formula (II); and isolating the compound of Formula (II).

In certain embodiments, the compound of Formula (II) is isolated as a solid.

In certain embodiments, the solvent added in the purification is acetone or isopropyl alcohol. In certain embodiments, the solvent added in the purification is acetone. In certain embodiments, the solvent added in the purification is isopropyl alcohol. In certain embodiments, the solvent added in the purification is cooled to a temperature that is below room temperature prior to adding the solvent. In certain embodiments, the solvent added in the purification is at a temperature that is equal to or below 30° C., 25° C., 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C. In certain embodiments, the solvent added in the purification is at a temperature of about 30° C., 25° C., 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C. In certain embodiments, the solvent added in the purification is at a temperature that is equal to or below room temperature. In certain embodiments, the solvent added in the purification is at room temperature. In certain embodiments, the solvent added in the purification is at a temperature that is equal to or below 0° C. In certain embodiments, the solvent added in the purification is at a temperature of about 0° C. In certain embodiments, the solvent added in the purification is at a temperature that is equal to or below −80° C. In certain embodiments, the solvent added in the purification is at a temperature of about −80° C.

In certain embodiments, purifying the compound of Formula (II) further comprises, after adding the purification solvent, cooling the reaction mixture to a temperature that is equal to or below room temperature. In certain embodiments, purifying the compound of Formula (II) further comprises, after adding the purification solvent, cooling the reaction mixture to a temperature that is equal to or below 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C. In certain embodiments, purifying the compound of Formula (II) further comprises, after adding the purification solvent, cooling the reaction mixture to a temperature of about 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C.

In certain embodiments, purifying the compound of Formula (II) further comprises adding a volume of an aqueous solution of a salt that is less than 1, 0.9, 0.8, 0.7, 0.6, 0.5, 0.4, 0.3, 0.2, or 0.1 times the amount of the total volume of a reaction mixture comprising the compound of Formula (II). In certain embodiments, purifying the compound of Formula (II) further comprises adding a volume of an aqueous solution of a salt that is about 1, 0.9, 0.8, 0.7, 0.6, 0.5, 0.4, 0.3, 0.2, or 0.1 times the amount of the total volume of a reaction mixture comprising the compound of Formula (II). In certain embodiments, purifying the compound of Formula (II) further comprises adding a volume of an aqueous solution of a salt that is about 0.1 times the amount of the total volume of a reaction mixture comprising the compound of Formula (II). In certain embodiments, purifying the compound of Formula (II) further comprises adding a volume of a 3M aqueous solution of a salt that is about 0.1 times the amount of the total volume of a reaction mixture comprising the compound of Formula (II). In certain embodiments, the salt is an alkali halide. In certain embodiments, the salt is LiCl, KCl, or NaCl. In certain embodiments, the salt is NaCl.

In certain embodiments wherein R is a charge-neutral oligonucleotide (e.g., phosphorodiamidate morpholino oligomer (PMO)), purifying the compound of Formula (II) comprises adding a volume of a solvent that is about 8 times the amount of the total volume of a reaction mixture comprising the compound of Formula (II); and isolating the compound of Formula (II). In certain embodiments wherein R is a charge-neutral oligonucleotide (e.g., phosphorodiamidate morpholino oligomer (PMO)), purifying the compound of Formula (II) comprises adding a volume of acetone or isopropyl alcohol that is about 8 times the amount of the total volume of a reaction mixture comprising the compound of Formula (II); and isolating the compound of Formula (II). In certain embodiments wherein R is a charge-neutral oligonucleotide (e.g., phosphorodiamidate morpholino oligomer (PMO)), purifying the compound of Formula (II) comprises adding a volume of acetone or isopropyl alcohol that is about 8 times the amount of the total volume of a reaction mixture comprising the compound of Formula (II); and isolating the compound of Formula (II); wherein the acetone or isopropyl alcohol is cooled to a temperature that is equal to or below 0° C. prior to adding the solvent. In certain embodiments wherein R is a charge-neutral oligonucleotide (e.g., phosphorodiamidate morpholino oligomer (PMO)), purifying the compound of Formula (II) comprises adding a volume of acetone that is about 8 times the amount of the total volume of a reaction mixture comprising the compound of Formula (II); and isolating the compound of Formula (II); wherein the acetone is cooled to a temperature that is equal to or below 0° C. prior to adding the solvent. In certain embodiments wherein R is a charge-neutral oligonucleotide (e.g., phosphorodiamidate morpholino oligomer (PMO)), purifying the compound of Formula (II) comprises adding a volume of isopropyl alcohol that is about 8 times the amount of the total volume of a reaction mixture comprising the compound of Formula (II); and isolating the compound of Formula (II); wherein the isopropyl alcohol is cooled to a temperature that is equal to or below 0° C. prior to adding the solvent.

In certain embodiments wherein R is a charged oligonucleotide, purifying the compound of Formula (II) comprises adding a volume of a solvent that is about 3 times the amount of the total volume of a reaction mixture comprising the compound of Formula (II); adding a volume of an aqueous solution of a salt that is about 0.1 times the amount of the total volume of a reaction mixture comprising the compound of Formula (II); and isolating the compound of Formula (II). In certain embodiments wherein R is a charged oligonucleotide, purifying the compound of Formula (II) comprises adding a volume of acetone or isopropyl alcohol that is about 3 times the amount of the total volume of a reaction mixture comprising the compound of Formula (II); adding a volume of a 3M aqueous solution of a salt that is about 0.1 times the amount of the total volume of a reaction mixture comprising the compound of Formula (II); and isolating the compound of Formula (II). In certain embodiments wherein R is a charged oligonucleotide, purifying the compound of Formula (II) comprises adding a volume of acetone or isopropyl alcohol that is about 3 times the amount of the total volume of a reaction mixture comprising the compound of Formula (II); adding a volume of a 3M aqueous solution of a salt that is about 0.1 times the amount of the total volume of a reaction mixture comprising the compound of Formula (II); and isolating the compound of Formula (II); wherein the acetone or isopropyl alcohol is cooled to a temperature that is equal to or below 0° C. prior to adding the solvent. In certain embodiments wherein R is a charged oligonucleotide, purifying the compound of Formula (II) comprises adding a volume of acetone that is about 3 times the amount of the total volume of a reaction mixture comprising the compound of Formula (II); adding a volume of a 3M aqueous solution of NaCl that is about 0.1 times the amount of the total volume of a reaction mixture comprising the compound of Formula (II); and isolating the compound of Formula (II); wherein the acetone is cooled to a temperature that is equal to or below 0° C. prior to adding the solvent. In certain embodiments wherein R is a charged oligonucleotide, purifying the compound of Formula (II) comprises adding a volume of isopropyl alcohol that is about 3 times the amount of the total volume of a mixture comprising the compound of Formula (II); adding a volume of a 3M aqueous solution of NaCl that is about 0.1 times the amount of the total volume of a mixture comprising the compound of Formula (II); and isolating the compound of Formula (II); wherein the isopropyl alcohol is cooled to a temperature that is equal to or below 0° C. prior to adding the solvent.

In certain embodiments wherein R is a charged oligonucleotide, purifying the compound of Formula (II) comprises adding a volume of acetone or isopropyl alcohol that is about 3 times the amount of the total volume of a reaction mixture comprising the compound of Formula (II); adding a volume of a 3M aqueous solution of NaCl that is about 0.1 times the amount of the total volume of a mixture comprising the compound of Formula (II); cooling to a temperature of about −80° C.; and isolating the compound of Formula (II); wherein the acetone or isopropyl alcohol is cooled to a temperature that is equal to or below 0° C. prior to adding the solvent. In certain embodiments wherein R is a charged oligonucleotide, purifying the compound of Formula (II) comprises adding a volume of acetone that is about 3 times the amount of the total volume of a reaction mixture comprising the compound of Formula (II); adding a volume of a 3M aqueous solution of NaCl that is about 0.1 times the amount of a reaction mixture comprising the compound of Formula (II); cooling the reaction mixture to a temperature of about −80° C.; and isolating the compound of Formula (II); wherein the acetone is cooled to a temperature that is equal to or below 0° C. prior to adding the solvent. In certain embodiments wherein R is a charged oligonucleotide, purifying the compound of Formula (II) comprises adding a volume of isopropyl alcohol that is about 3 times the amount of the total volume of a reaction mixture comprising the compound of Formula (II); adding a volume of a 3M aqueous solution of NaCl that is about 0.1 times the amount of the total volume of a reaction mixture comprising the compound of Formula (II); cooling the reaction mixture to a temperature of about −80° C.; and isolating the compound of Formula (II); wherein the isopropyl alcohol is cooled to a temperature that is equal to or below 0° C. prior to adding the solvent.

In certain embodiments of the purification of the compound of Formula (II), the mixture comprising the compound of Formula (II) is or comprises the completed reaction of the compound of Formula (III) with the compound of Formula (IV). Thus, in certain embodiments, purifying the compound of Formula (II) comprises adding, to the reaction mixture comprising the completed reaction of the compound of Formula (III) with the compound of Formula (IV), a volume of a solvent that is at least 2, 3, 4, 5, 6, 7, 8, 9, or 10 times the amount of the total volume of the reaction mixture of the compound of Formula (III) with the compound of Formula (IV); and isolating the compound of Formula (II) from the reaction mixture. In certain embodiments, purifying the compound of Formula (II) comprises adding, to the reaction mixture comprising the completed reaction of the compound of Formula (III) with the compound of Formula (IV), a volume of a solvent that is at least 8 times the amount of the total volume of the reaction mixture of the compound of Formula (III) with the compound of Formula (IV); and isolating the compound of Formula (II) from the reaction mixture. In certain embodiments, purifying the compound of Formula (II) comprises adding, to the reaction mixture comprising the completed reaction of the compound of Formula (III) with the compound of Formula (IV), a volume of a solvent that is at least 3 times the amount of the total volume of the reaction mixture of the compound of Formula (III) with the compound of Formula (IV); and isolating the compound of Formula (II) from the reaction mixture. In certain embodiments, purifying the compound of Formula (II) comprises adding, to the completed reaction mixture of the compound of Formula (III) with the compound of Formula (IV), a volume of a solvent that is about 2, 3, 4, 5, 6, 7, 8, 9, or 10 times the amount of the total volume of the reaction mixture of the compound of Formula (III) with the compound of Formula (IV); and isolating the compound of Formula (II) from the reaction mixture. In certain embodiments, purifying the compound of Formula (II) comprises adding, to the reaction mixture comprising the completed reaction of the compound of Formula (III) with the compound of Formula (IV), a volume of a solvent that is about 8 times the amount of the total volume of the reaction mixture of the compound of Formula (III) with the compound of Formula (IV); and isolating the compound of Formula (II) from the reaction mixture. In certain embodiments, purifying the compound of Formula (II) comprises adding, to the reaction mixture comprising the completed reaction of the compound of Formula (III) with the compound of Formula (IV), a volume of a solvent that is about 3 times the amount of the total volume of the reaction mixture of the compound of Formula (III) with the compound of Formula (IV); and isolating the compound of Formula (II) from the reaction mixture.

In certain embodiments, the compound of Formula (II) is isolated as a solid.

In certain embodiments, the solvent added in the purification is acetone or isopropyl alcohol. In certain embodiments, the solvent added in the purification is acetone. In certain embodiments, the solvent added in the purification is isopropyl alcohol. In certain embodiments, the solvent added in the purification is cooled to a temperature that is below room temperature prior to adding the solvent. In certain embodiments, the solvent added in the purification is at a temperature that is equal to or below 30° C., 25° C., 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C. In certain embodiments, the solvent added in the purification is at a temperature of about 30° C., 25° C., 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C. In certain embodiments, the solvent added in the purification is at a temperature that is equal to or below room temperature. In certain embodiments, the solvent added in the purification is at room temperature. In certain embodiments, the solvent added in the purification is at a temperature that is equal to or below 0° C. In certain embodiments, the solvent added in the purification is at a temperature of about 0° C. In certain embodiments, the solvent added in the purification is at a temperature that is equal to or below −80° C. In certain embodiments, the solvent added in the purification is at a temperature of about −80° C.

In certain embodiments, purifying the compound of Formula (II) further comprises, after adding the purification solvent, cooling the reaction mixture to a temperature that is equal to or below room temperature. In certain embodiments, purifying the compound of Formula (II) further comprises, after adding the purification solvent, cooling the reaction mixture to a temperature that is equal to or below 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C. In certain embodiments, purifying the compound of Formula (II) further comprises, after adding the purification solvent, cooling the reaction mixture to a temperature of about 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C.

In certain embodiments, purifying the compound of Formula (II) further comprises adding, to the reaction mixture comprising the completed reaction of the compound of Formula (III) with the compound of Formula (IV), a volume of an aqueous solution of a salt that is less than 1, 0.9, 0.8, 0.7, 0.6, 0.5, 0.4, 0.3, 0.2, or 0.1 times the amount of the total volume of the reaction mixture of the compound of Formula (III) with the compound of Formula (IV). In certain embodiments, purifying the compound of Formula (II) further comprises adding, to the reaction mixture comprising the completed reaction of the compound of Formula (III) with the compound of Formula (IV), a volume of an aqueous solution of a salt that is about 1, 0.9, 0.8, 0.7, 0.6, 0.5, 0.4, 0.3, 0.2, or 0.1 times the amount of the total volume of the reaction mixture of the compound of Formula (III) with the compound of Formula (IV). In certain embodiments, purifying the compound of Formula (II) further comprises adding, to the reaction mixture comprising the completed reaction of the compound of Formula (III) with the compound of Formula (IV), a volume of an aqueous solution of a salt that is about 0.1 times the amount of the total volume of the reaction mixture of the compound of Formula (III) with the compound of Formula (IV). In certain embodiments, purifying the compound of Formula (II) further comprises adding, to the reaction mixture comprising the completed reaction of the compound of Formula (III) with the compound of Formula (IV), a volume of a 3M aqueous solution of a salt that is about 0.1 times the amount of the total volume of the reaction mixture of the compound of Formula (III) with the compound of Formula (IV). In certain embodiments, the salt is an alkali halide. In certain embodiments, the salt is LiCl, KCl, or NaCl. In certain embodiments, the salt is NaCl.

In certain embodiments wherein R is a charge-neutral oligonucleotide (e.g., phosphorodiamidate morpholino oligomer (PMO)), purifying the compound of Formula (II) comprises adding, to the reaction mixture comprising the completed reaction of the compound of Formula (III) with the compound of Formula (IV), a volume of a solvent that is about 8 times the amount of the total volume of the reaction mixture of the compound of Formula (III) with the compound of Formula (IV); and isolating the compound of Formula (II) from the reaction mixture. In certain embodiments wherein R is a charge-neutral oligonucleotide (e.g., phosphorodiamidate morpholino oligomer (PMO)), purifying the compound of Formula (II) comprises adding, to the reaction mixture comprising the completed reaction of the compound of Formula (III) with the compound of Formula (IV), a volume of acetone or isopropyl alcohol that is about 8 times the amount of the total volume of the reaction mixture of the compound of Formula (III) with the compound of Formula (IV); and isolating the compound of Formula (II) from the reaction mixture. In certain embodiments wherein R is a charge-neutral oligonucleotide (e.g., phosphorodiamidate morpholino oligomer (PMO)), purifying the compound of Formula (II) comprises adding, to the reaction mixture comprising the completed reaction of the compound of Formula (III) with the compound of Formula (IV), a volume of acetone or isopropyl alcohol that is about 8 times the amount of the total volume of the reaction mixture of the compound of Formula (III) with the compound of Formula (IV); and isolating the compound of Formula (II) from the reaction mixture; wherein the acetone or isopropyl alcohol is cooled to a temperature that is equal to or below 0° C. prior to adding the solvent. In certain embodiments wherein R is a charge-neutral oligonucleotide (e.g., phosphorodiamidate morpholino oligomer (PMO)), purifying the compound of Formula (II) comprises adding, to the reaction mixture comprising the completed reaction of the compound of Formula (III) with the compound of Formula (IV), a volume of acetone that is about 8 times the amount of the total volume of the reaction mixture of the compound of Formula (III) with the compound of Formula (IV); and isolating the compound of Formula (II) from the reaction mixture; wherein the acetone is cooled to a temperature that is equal to or below 0° C. prior to adding the solvent. In certain embodiments wherein R is a charge-neutral oligonucleotide (e.g., phosphorodiamidate morpholino oligomer (PMO)), purifying the compound of Formula (II) comprises adding, to the reaction mixture comprising the completed reaction of the compound of Formula (III) with the compound of Formula (IV), a volume of isopropyl alcohol that is about 8 times the amount of the total volume of the reaction mixture of the compound of Formula (III) with the compound of Formula (IV); and isolating the compound of Formula (II) from the reaction mixture; wherein the isopropyl alcohol is cooled to a temperature that is equal to or below 0° C. prior to adding the solvent.

In certain embodiments wherein R is a charged oligonucleotide, purifying the compound of Formula (II) comprises adding, to the reaction mixture comprising the completed reaction of the compound of Formula (III) with the compound of Formula (IV), a volume of a solvent that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of an aqueous solution of a salt that is about 0.1 times the amount of the total volume of the reaction mixture of the compound of Formula (III) with the compound of Formula (IV); and isolating the compound of Formula (II) from the reaction mixture. In certain embodiments wherein R is a charged oligonucleotide, purifying the compound of Formula (II) comprises adding, to the reaction mixture comprising the completed reaction of the compound of Formula (III) with the compound of Formula (IV), a volume of acetone or isopropyl alcohol that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of a 3M aqueous solution of a salt that is about 0.1 times the amount of the total volume of the reaction mixture of the compound of Formula (III) with the compound of Formula (IV); and isolating the compound of Formula (II) from the reaction mixture. In certain embodiments wherein R is a charged oligonucleotide, purifying the compound of Formula (II) comprises adding, to the reaction mixture comprising the completed reaction of the compound of Formula (III) with the compound of Formula (IV), a volume of acetone or isopropyl alcohol that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of a 3M aqueous solution of a salt that is about 0.1 times the amount of the total volume of the reaction mixture of the compound of Formula (III) with the compound of Formula (IV); and isolating the compound of Formula (II) from the reaction mixture; wherein the acetone or isopropyl alcohol is cooled to a temperature that is equal to or below 0° C. prior to adding the solvent. In certain embodiments wherein R is a charged oligonucleotide, purifying the compound of Formula (II) comprises adding, to the reaction mixture comprising the completed reaction of the compound of Formula (III) with the compound of Formula (IV), a volume of acetone that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of a 3M aqueous solution of NaCl that is about 0.1 times the amount of the total volume of the reaction mixture of the compound of Formula (III) with the compound of Formula (IV); and isolating the compound of Formula (II) from the reaction mixture; wherein the acetone is cooled to a temperature that is equal to or below 0° C. prior to adding the solvent. In certain embodiments wherein R is a charged oligonucleotide, purifying the compound of Formula (II) comprises adding, to the reaction mixture comprising the completed reaction of the compound of Formula (III) with the compound of Formula (IV), a volume of isopropyl alcohol that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of a 3M aqueous solution of NaCl that is about 0.1 times the amount of the total volume of the reaction mixture of the compound of Formula (III) with the compound of Formula (IV); and isolating the compound of Formula (II) from the reaction mixture; wherein the isopropyl alcohol is cooled to a temperature that is equal to or below 0° C. prior to adding the solvent.

In certain embodiments wherein R is a charged oligonucleotide, purifying the compound of Formula (II) comprises adding, to the reaction mixture comprising the completed reaction of the compound of Formula (III) with the compound of Formula (IV), a volume of acetone or isopropyl alcohol that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of a 3M aqueous solution of a salt that is about 0.1 times the amount of the total volume of the reaction mixture of the compound of Formula (III) with the compound of Formula (IV); cooling the reaction mixture to a temperature of about −80° C.; and isolating the compound of Formula (II) from the reaction mixture; wherein the acetone or isopropyl alcohol is cooled to a temperature that is equal to or below 0° C. prior to adding the solvent. In certain embodiments wherein R is a charged oligonucleotide, purifying the compound of Formula (II) comprises adding, to the reaction mixture comprising the completed reaction of the compound of Formula (III) with the compound of Formula (IV), a volume of acetone that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of a 3M aqueous solution of NaCl that is about 0.1 times the amount of the total volume of the reaction mixture of the compound of Formula (III) with the compound of Formula (IV); cooling the reaction mixture to a temperature of about −80° C.; and isolating the compound of Formula (II) from the reaction mixture; wherein the acetone is cooled to a temperature that is equal to or below 0° C. prior to adding the solvent. In certain embodiments wherein R is a charged oligonucleotide, purifying the compound of Formula (II) comprises adding, to the reaction mixture comprising the completed reaction of the compound of Formula (III) with the compound of Formula (IV), a volume of isopropyl alcohol that is about 3 times the amount of the total volume of the reaction mixture of the compound of Formula (III) with the compound of Formula (IV); adding, to the reaction mixture comprising the completed reaction of the compound of Formula (III) with the compound of Formula (IV), a volume of a 3M aqueous solution of NaCl that is about 0.1 times the amount of the total volume of the reaction mixture of the compound of Formula (III) with the compound of Formula (IV); cooling the reaction mixture to a temperature of about −80° C.; and isolating the compound of Formula (II) from the reaction mixture; wherein the isopropyl alcohol is cooled to a temperature that is equal to or below 0° C. prior to adding the solvent.

### V. Pharmaceutical Compositions and Administration

The present disclosure provides pharmaceutical compositions comprising a compound of Formula (I), or a pharmaceutically acceptable salt, tautomer, stereoisomer, or isotopically enriched derivative, and optionally a pharmaceutically acceptable excipient. In certain embodiments, the pharmaceutical composition described herein comprises a compound of Formula (I), or a pharmaceutically acceptable salt thereof, and a pharmaceutically acceptable excipient.

In certain embodiments, the compound of Formula (I) is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a muscle disease in a subject in need thereof.

In some embodiments, the disclosure provides compositions that are useful for delivering payloads to target cells. The present disclosure provides compositions that are useful in methods of treating a disease or condition in a subject in need thereof. For example, the methods may involve administering to a subject a compound of Formula (I), or a pharmaceutically acceptable salt, tautomer, stereoisomer, or isotopically enriched derivative thereof, or a pharmaceutical composition disclosed herein.

In some embodiments, the compound comprises a muscle-targeting agent, e.g. an anti-transferrin receptor antibody, and an antisense oligonucleotide that targets a muscle disease allele.

In some embodiments, the methods are useful for treating a muscle disease, in which a molecular payload affects the activity of the corresponding gene provided in Table 2. For example, depending on the condition, a molecular payload may modulate (e.g., decrease, increase) transcription or expression of the gene, modulate the expression of a protein encoded by the gene, or to modulate the activity of the encoded protein. In some embodiments, the molecular payload is an oligonucleotide that comprises a strand having a region of complementarity to a target gene provided in Table 2. In some embodiments, the methods are useful for treating any muscle disease listed in Table 2.

TABLE 2
List of muscle diseases and corresponding genes.

| | Gene | |
|---|---|---|
| Disease | Symbol | GenBank Accession No. |
| Adult Pompe | GAA | NM_000152; NM_001079803; |
| | | NM_001079804 |
| Adult Pompe | GYS1 | NM_001161587; NM_002103 |
| Centronuclear myopathy (CNM) | DNM2 | NM_001190716; NM_004945; |
| | | NM_001005362; NM_001005360; |
| | | NM_001005361; NM_007871 |
| Cardiac fibrosis, Cardiac | ACVR1B | NM_004302.5, NM_020327.3, |
| hypertrophy | | NM_020328.4 |
| cardiac hypertrophy, cardiac fibrosis, | KLF15 | NM_014079.4 |
| arrhythmia, | | |
| congenital heart disease, congenital | MED1 | NM_004774.4 |
| heart defect | | |
| congenital heart disease, congenital | MED13 | NM_005121.3 |
| heart defect | | |
| Duchenne muscular dystrophy, | DMD | NM_004023; NM_004020; |
| dystrophinopathy | | NM_004018; NM_004012; |
| | | NM_000109.3, NM_004006.2, |
| | | NM_004009.3, NM_004010.3, |
| | | NM_004011.3 |
| Facioscapulohumeral muscular | DUX4 | NM_001306068; NM_001363820; |
| dystrophy (FSHD) | | NM_001205218; NM_001293798 |
| cardiomyopathy, muscle atrophy, | INHBA | NM_002192.4 |
| muscular dystrophy, cardiac cachexia | | |
| Irritable bowel syndrome (IBS); | MLCK1 | NM_001321309.2; NM_053025.4; |
| Inflammatory bowel disease (IBD) | | NM_053026.4; NM_053027.4; |
| | | NM_053028.4; NM_053031.4; |
| | | NM_053032.4 |
| Cardiac muscle wasting, | MSTN | NM_005259.3 |
| Cardiomyopathy, Cardiac cachexia, | | |
| Skeletal muscle atrophy | | |
| Familial hypertrophic | MYBPC3 | NM_000256 |
| cardiomyopathy | | |
| Familial hypertrophic | MYH6 | NM_002471; NM_001164171; |
| cardiomyopathy | | NM_010856 |
| Familial hypertrophic | MYH7 | NM_000257; NM_080728 |
| cardiomyopathy | | |
| Familial hypertrophic | TNNI3 | NM_000363 |
| cardiomyopathy | | |
| Familial hypertrophic | TNNT2 | NM_001001432; NM_001001431; |
| cardiomyopathy | | NM_000364; NM_001001430; |
| | | NM_001276347; NM_001276346; |
| | | NM_001276345 |
| Fibrodysplasia Ossificans | ACVR1 | NM_001105; NM_001347663; |
| Progressiva (FOP), Cardiac | | NM_001347664; NM_001347665; |
| hypertrophy, Muscle atrophy | | NM_001347666; NM_001347667; |
| | | NM_001111067 |
| Friedreich's ataxia (FRDA) | FXN | NM_001161706; NM_181425; |
| | | NM_000144 |
| heart failure | PPP1R3A | NM_002711.4 |
| Inclusion body myopathy 2 | GNE | NM_001190383; NM_001190384; |
| | | NM_001128227; NM_005476; |
| | | NM_001190388 |
| Laing distal myopathy | MYH7 | NM_000257; NM_080728 |
| muscle atrophy | FBXO32 | NM_058229.4, NM_001242463.2, |
| | | NM_148177.2 |
| muscle atrophy | TRIM63 | NM_032588.3 |
| muscle atrophy, myotonic dystrophy, | MEF2D | NM_001271629.2, NM_005920.4 |
| cardiac hypertrophy, | | |
| cardiomyopathy, Parkinson's | | |
| disease, amyotrophic lateral sclerosis | | |
| (ALS) | | |
| Myofibrillar myopathy | BAG3 | NM_004281 |
| Myofibrillar myopathy | CRYAB | NM_001885; NM_001330379; |
| | | NM_001289807; NM_001289808 |
| Myofibrillar myopathy | DES | NM_001927 |
| Myofibrillar myopathy | DNAJB6 | NM_005494; NM_058246 |
| Myofibrillar myopathy | FHL1 | NM_001159701; NM_001159699; |
| | | NM_001159702; NM_001159703; |
| | | NM_001159704; NM_001159700; |
| | | NM_001167819; NM_001330659; |
| | | NM_001449; NM_001077362 |
| Myofibrillar myopathy | FLNC | NM_001458; NM_001127487 |
| Myofibrillar myopathy | LDB3 | NM_007078; NM_001171611; |
| | | NM_001171610; NM_001080114; |
| | | NM_001080115; NM_001080116 |
| Myofibrillar myopathy | MYOT | NM_001300911; NM_006790; |
| | | NM_001135940 |
| Myofibrillar myopathy | PLEC | NM_201378; NM_201379; |
| | | NM_201380; NM_201381; |
| | | NM_201382; NM_201383; |
| | | NM_201384; NM_000445 |
| Myofibrillar myopathy | TTN | NM_133432; NM_133379; |
| | | NM_133437; NM_003319; |
| | | NM_001256850; NM_001267550; |
| | | NM_133378 |
| Myotonia congenita (autosomal | CLCN1 | NM_000083; NM_013491 |
| dominant form, Thomsen Disease) | | |
| Myotonic dystrophy type I | DMPK | NM_001081563; NM_004409; |
| | | NM_001081560; NM_001081562; |
| | | NM_001288764; NM_001288765; |
| | | NM_001288766 |
| Myotonic dystrophy type II | CNBP | NM_001127192; NM_001127193; |
| | | NM_001127194; NM_001127195; |
| | | NM_001127196; NM_003418 |
| Myotubular myopathy | MTM1 | NM_000252 |
| Oculopharyngeal muscular dystrophy | PABPN1 | NM_004643 |
| Paramyotonia congenita | SCN4A | NM_000334 |

### Examples

In order that the invention described herein may be more fully understood, the following examples are set forth. The examples described in this application are offered to illustrate the compounds, pharmaceutical compositions, and methods provided herein and are not to be construed in any way as limiting their scope.

### Preparation of Compound A

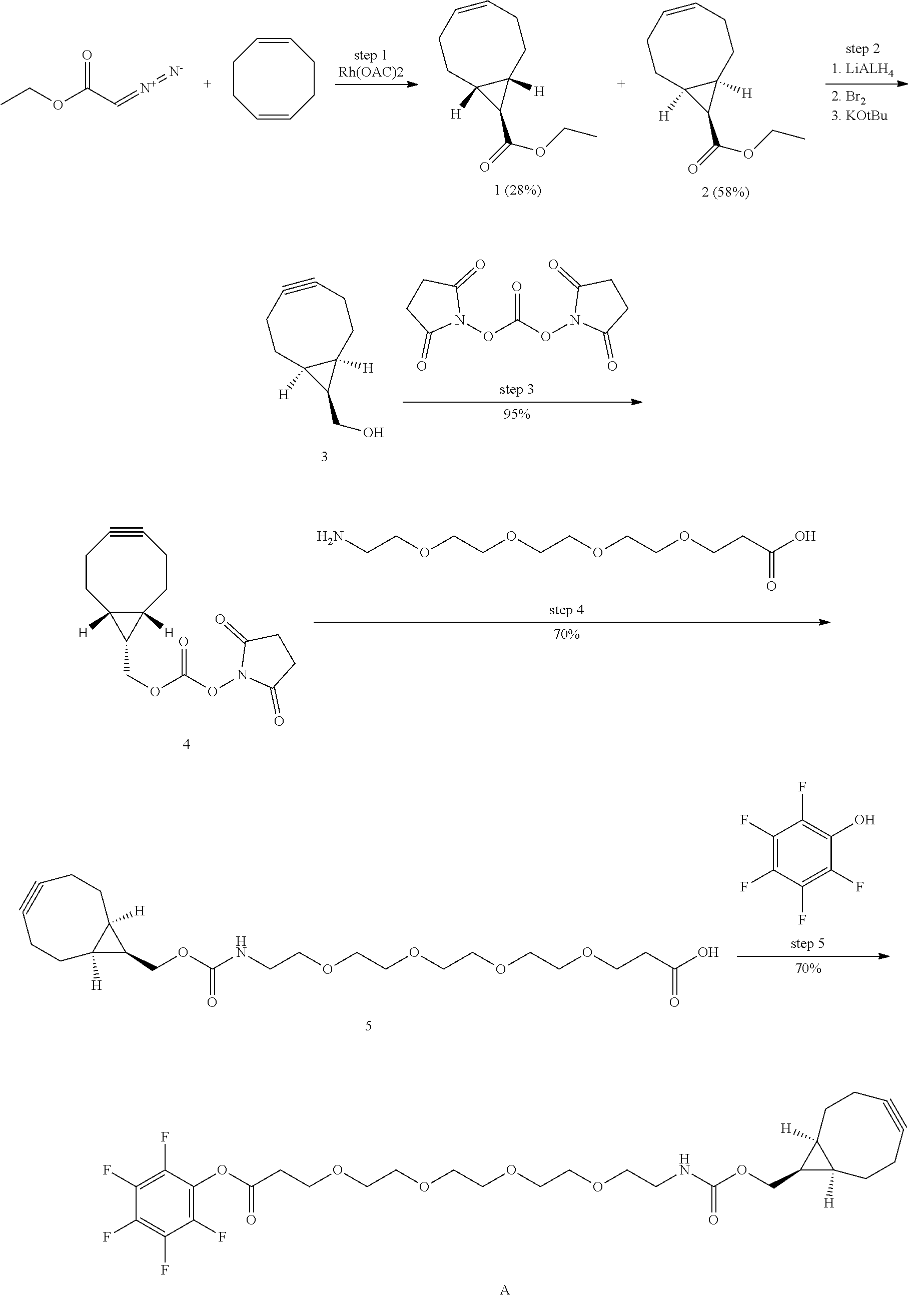

Step 1: Cyclooctadiene (8 eq.) and Rh₂(OAc)₄ (0.08 eq.) were charged into jacket reactor and heated to 60° C. Ethyl diazoacetate (1 eq.) in DCM (29 vol) was charged dropwise to keep the temperature at 60° C. After charging, the reaction mixture was cooled to 25° C. and stirred until conversion was more than 99% by GC. The mixture was concentrated to remove cyclooctadiene, then purified by silica column. The endo product (Compound 2) was obtained as oil after evaporating the solvent.

Step 2: THF (10 vol) was charged into reactor and then cooled to 0° C. LiAlH₄ (1.2 eq.) was slowly charged into the reactor. Compound 2 in THF (10 vol) was slowly charged while keeping the temperature below 25° C. After the charging was finished, the reaction mixture was warmed to 25° C. and stirred until reaction complete. The mixture was cooled to 5° C. and then saturated sodium sulfate was slowly charged. The mixture was allowed to warm to 25° C. and stirred for 2 h. The suspension was filtered through celite and washed with THF (2 vol). The mixture was concentrated to an oil.

The oil was dissolved in DCM (15 vol) and then cooled to −5° C. The Br₂ (1.2 eq.) in DCM (7 vol) was charged slowly until the solution color changed. The reaction was stirred until the IPC for conversion passed. The mixture was then quenched with 10% Na₂SO₃. The phases were separated, and the aqueous phase was extracted with DCM. The combined organic phase was concentrated to a solid. The product was reslurred with heptane. After filtration, the product was dried at room temperature under vacuum.

The solid was charged into THF (5 vol) and then cooled to 0° C. 1M t-BuOK (3.5 eq.) in THF was charged and the mixture was heated to 65° C. After about 1 hour, IPC conversion was more than 99%. The mixture was cooled to room temperature, and quenched with 10% aq. NH₄Cl (5 vol) and EA (5 vol). The organic layer was separated and concentrated. The oil was purified by silica column with eluent of EA and PE. The eluent was evaporated and then EA was charged in to dissolve the product. Heptane was charged and then distilled to exchange EA. Finally, heptane was charged and the suspension was filtered. The product was dried under vacuum to provide Compound 3.

Step 3: Compound 3 (1 eq.) was charged into a reactor. Acetonitrile (10.5 vol) and trimethylamine (3 eq.) were added and then the mixture was cooled to 0° C. N,N′-disuccinimidyl carbonate (1.8 eq.) was charged slowly. After that, the temperature was raised to room temperature and stirred for 10 hours. After IPC for conversion had passed, the mixture was concentrated and then DCM (8 vol) was charged. After filtration, the cake was washed with DCM (1.5 vol). The filtrate was concentrated and purified by silica column with eluent of EA and heptane. Compound 4 was obtained as a solid product after evaporation.

Step 4: 1-Amino-3,6,9,12-tetraoxapentadecan-15-oic acid (1.2 eq.) was dissolved in DCM (5 volumes), then DIPEA (3 eq.) was added. Compound 4 (1 eq.) was dissolved in DCM (5 volumes) and added slowly. After the reaction was complete, aq. Na₂SO₄ and citric acid solution (10 volumes) were added. The organic phase was collected and the aqueous phase was washed with DCM (20 volumes). After phase separation, the combined organic extracts were concentrated and purified on silica gel to provide Compound 5.

The reagent amount, solvent amount, and time of the reaction may be varied within the range of 50-150%. The temperature can be in the range of the target temperature±10° C.

Step 5: Compound 5 (1 eq.) was dissolved in ethyl acetate (10 vol), and pentafluorophenol (2 eq.) was added. Then DIPEA (2.5 eq) was added, and pentafluorophenyl trifluoroacetate (1.5 eq) was added. The reaction was stirred at r.t. under a nitrogen atmosphere. After the reaction was complete, n-heptane (6 vol) and 0.15 N HCl (15 vol) were added. The organic phase was collected and the aqeuous phase washed with ethyl acetate (5 vol). After phase separation, the combine ethyl acetate extracts were washed with 0.15 N HCl (15 vol) and saturated brine (10 vol). The organic phase was collected, concentrated, and purified on silica gel to yield Compound A as a sticky oil.

The reagent/solvent amount, and time may be varied within the range of 50%-150%. The temperature can be in the range of target temperature±10° C.

### Preparation of Compound B

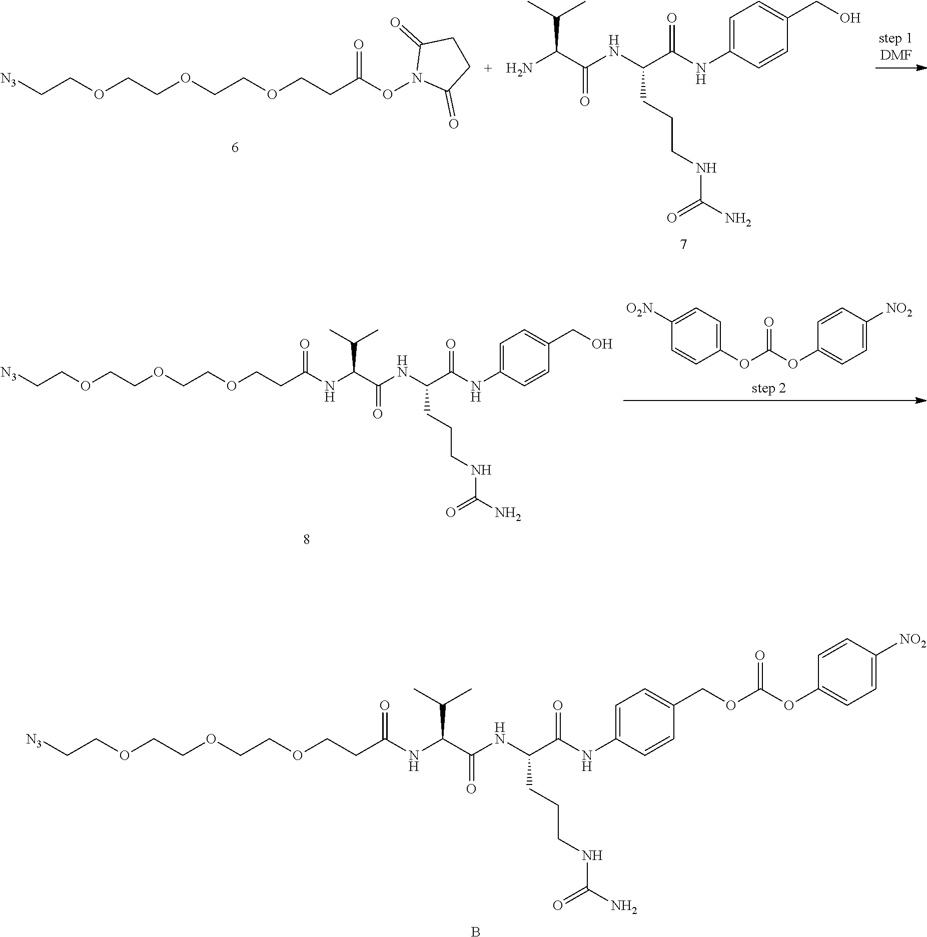

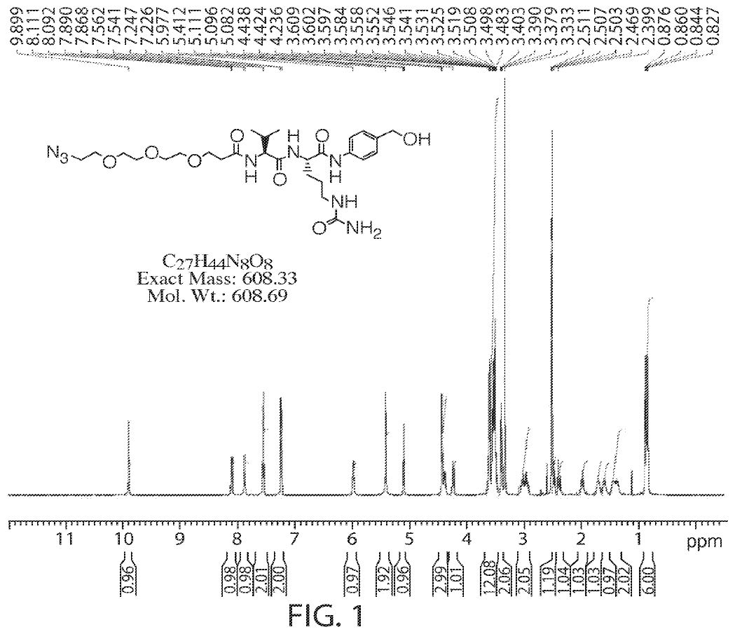

Step 1: Compound 7 (1.0 eq) and DIPEA (1.2 eq) were dissolved in DMF (4 volumes) at about 25° C. Then, Compound 6 (1.1 eq) was added to the mixture dropwise at 25° C. The mixture was further stirred at 25° C. for 4 hrs. Then the product was precipitated by charging MeCN (16 volumes) at 25° C. and a white solid was observed. The mixture was further cooled to −2° C. and aged for 2 hr. The solid was filtered and washed with cool MeCN to yield Compound 8. Product was dried at 30° C. and 40° C. The ¹H NMR spectrum of Compound 8 is shown in FIG. 1.

The reagent/solvent amount, and time may be varied with the range of 50%-150%. The temperature can be in the range of target temperature±10° C.

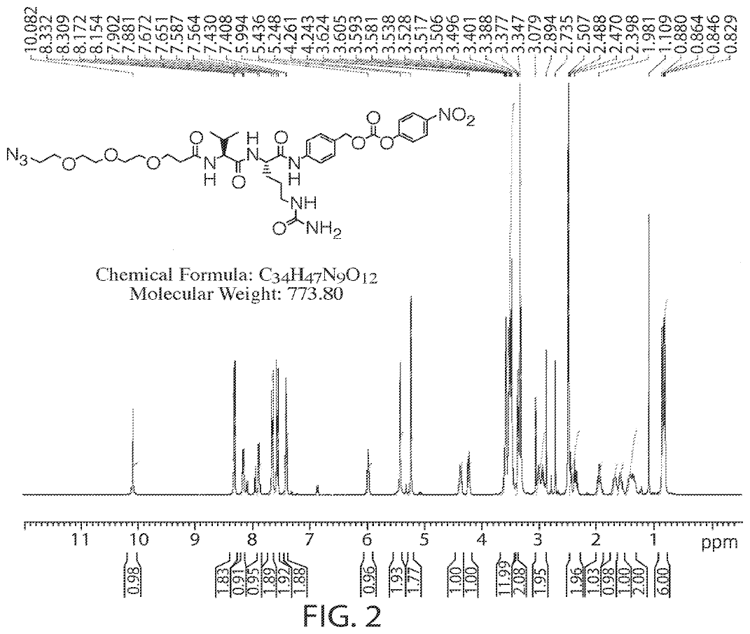

Step 2: Compound 8, bis(4-nitrophenyl) carbonate, and DIPEA were dissolved in DMF and acetone. The mixture was stirred at 25° C. until complete reaction. Then the mixture was precipitated from a mixture of MTBE and celite. The mixture was filtered and the filter cake washed with MTBE, acetone/MTBE, and acetone to purge impurities. Next, the mixture was washed with acetone/DMF at 40° C. to remove celite. The filter cake was further washed with acetone/DMF. The filtrate was combined and concentrated to remove acetone and the product precipitated with MTBE. The obtained filter cake was washed with MTBE and dried under vacuum at 25° C. to give Compound B. The ¹H NMR spectrum of Compound B is shown in FIG. 2.

The reagent/solvent amount, and time may be varied with the range of 50%-150%. The temperature can be in the range of target temperature±10° C.

### Preparation of Compound C

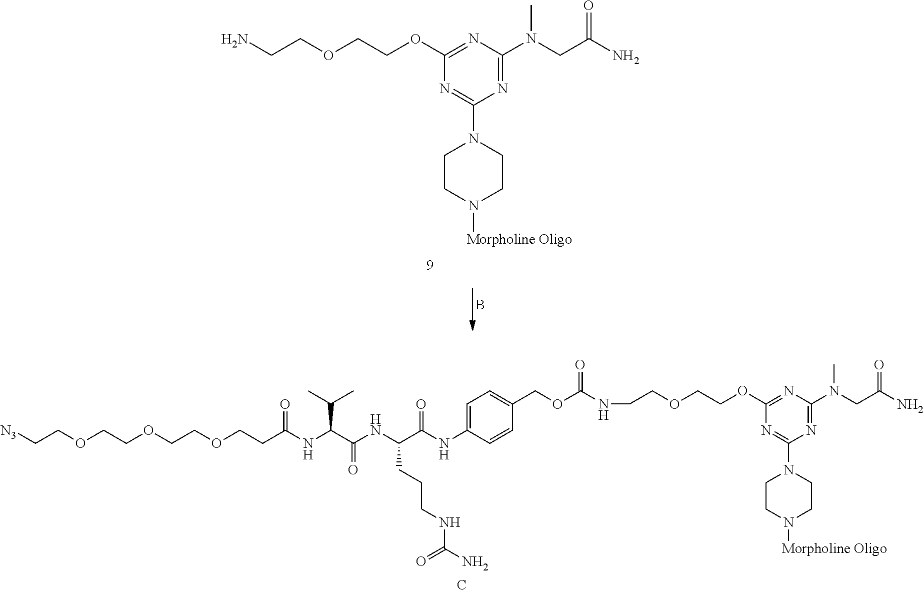

Compound 9 was dissolved at 35 mg/mL in anhydrous DMSO with heating to 37° C. for 10 minutes. In parallel, Compound B was dissolved at 40 mg/mL in anhydrous DMF. The solution of Compound B was then mixed with an appropriate volume of the Compound 9 solution (at a 2.7:1 mol:mol ratio of Compound 9 to Compound B) containing 3 molar equivalents of DIPEA with respect to Compound B. The solution was allowed to stir at room temperature for 2 h. Reaction completion was measured using ninhydrin (Kaiser test) in order to proceed to acetone precipitation.

After completion of the reaction was confirmed by the ninhydrin test, precipitation was accomplished by the addition of 8 volumes of chilled acetone to the crude reaction mixture. The precipitate was isolated by centrifugation at 3500×g at 8° C. for 20 minutes. After decanting the supernatant, the pellet was washed thoroughly with 3 volumes of acetone to remove unreacted Compound B and then centrifuged at 3500×g at 8° C. for 20 minutes. Then, the pellet was washed thoroughly with 3 volumes of acetonitrile and centrifuged at 3500×g for 20 minutes. After decanting the supernatant, the purified Compound C was re-dissolved in 20% v/v acetonitrile in nuclease free water at 30 mg/mL. Concentration and yield were measured by OD on aliquots of the solution containing a final concentration of 0.1N HCl. Yield for the synthesis of Compound C was >90% (m/z=11073.82). The product can be either used immediately for reaction or lyophilized for long term storage (>2 weeks). Purity and removal of residual Compound B was monitored by analytical RP-HPLC and LCMS.

Preparation of Compound C-1

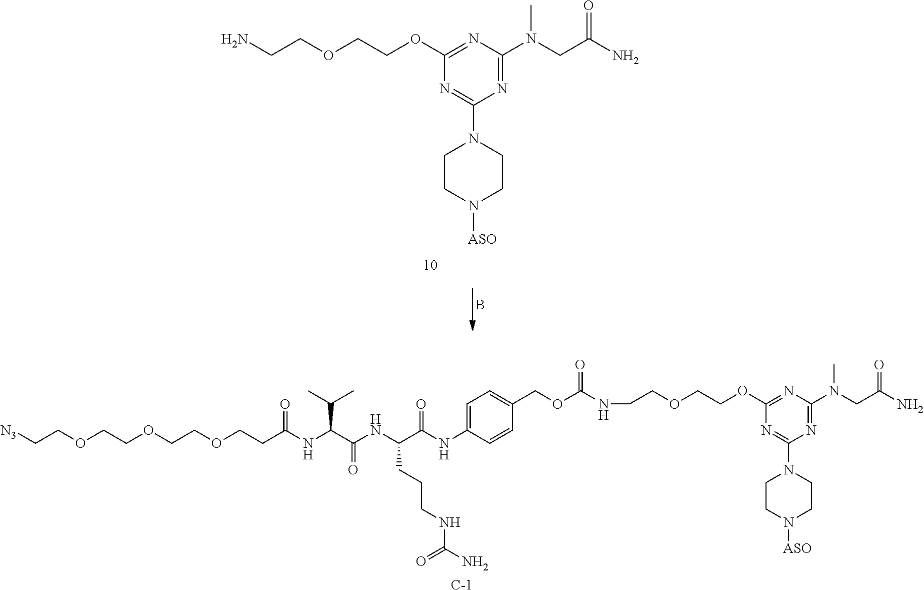

Compound 10 comprises a charged oligonucleotide. A 20 mg/mL (25.85 mM) stock solution of compound B was prepared in DMF. Lyophilized compound 10 (252 mg) was solubilized in a 15 mL falcon tube targeting a concentration of approximately 150 mg/mL by adding 1.3 mL of milli Q water. The tube was placed in a 37° C. water bath for 10 minutes to ensure the compound was completely dissolved as a clear solution. The concentration of the stock solution in water was determined with a Nanodrop UV/vis instrument by using an aliquot diluted 100-fold in water at 260 nm with an extinction coefficient of 141 mM⁻¹cm⁻¹ (24.93 mg⁻¹mLcm⁻¹). The resulting solution was 204.8 mg in total at 144.2 mg/ml (25.50 mM).

2.006 mM (204.8 mg; 1.42 mL of stock solution prepared above) compound 10, 6.018 mM compound B (1.0:3.0 mol:mol equivalents; 4.2 mL of stock solution prepared above), and 0.431 mL tributylamine (1:50 mol:mol equivalents) were combined in 92 v/v % DMF (12 mL) at room temperature.

The reaction was carried out at room temperature for 3 hours. Completion of the reaction was monitored by RP-C18 UPLC and LC-MS methods. Once the reaction was determined to be complete, 1.8 mL of 3M NaCl (1/10 volume of crude reaction mixture) and 54.2 mL of ice-cold IPA (3 volumes of crude reaction mixture) were added to the crude reaction mixture and the tube was cooled to −80° C. for more than 30 minutes. After centrifuging the tube at 4500 rpm for 20 minutes, supernatant was discarded. The pellet was washed with 75% ethanol (room temperature) twice. After all traces of ethanol were removed, 5 mL of 15% acetonitrile in water was added to the tube to reconstitute the pellet. Compound C-1 was isolated (198 mg; 96%).

### Preparation of Complexes—Method 2

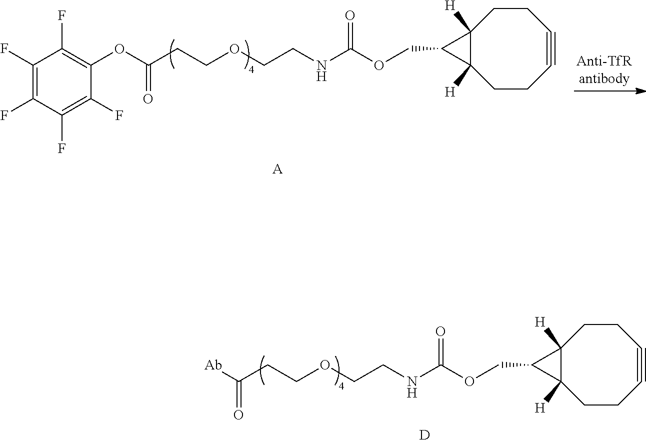

An anti-TfR Fab′ was diluted with propylene glycol to a final concentration of 40% v/v propylene glycol and incubated with 5-fold molar excess of Compound A dissolved in DMSO (at a concentration of 20 mg/mL) for 2 hr at room temperature (˜22.5° C.). It was anticipated that labeling should yield 2.0-2.5 moles of Compound A per mole of Fab′. Post labeling, the reaction product was sterile filtered or depth filtered to remove unreacted Compound A. The filtered solution was then assayed for average reactive BCN moieties analytically using LCMS (ThermoFisher MAbPac RP 4 um 2.1×100 mm, #088647; mobile phase A 0.1% formic acid in 100% UPLC-grade water; mobile phase B 0.1% formic acid in 100% UPLC-grade acetonitrile; flow rate 0.3 mL/min; column temperature 70° C.; in-source CID 20 eV; positive polarity; spray voltage 3.5 kV; scan range 1000-3000 m/z).

Compound D (containing anti-TfR with a degree of labeling (DOL) of >2.3) was taken to the next step of the conjugation, and was purified into 10% isopropanol in PBS at pH 7.2 by tangential flow filtration using a 10 kDa molecular weight cutoff (1.2 bar), with 5 filtrate volumes, to remove byproducts and propylene glycol. Complete removal of unreacted starting material and propylene glycol was verified by analytical HPLC-SEC (Waters Xbridge Protein BEH SEC 3.5 um, 7.8×300 mm, 0.3 mL/min, 100 mM PO₄, 100 mM NaCl, 15% v/v acetonitrile pH 7.0). The recovery of Compound D was >90% of starting material. The purified solution was concentrated to 3.5 mg/mL for further conjugation steps.

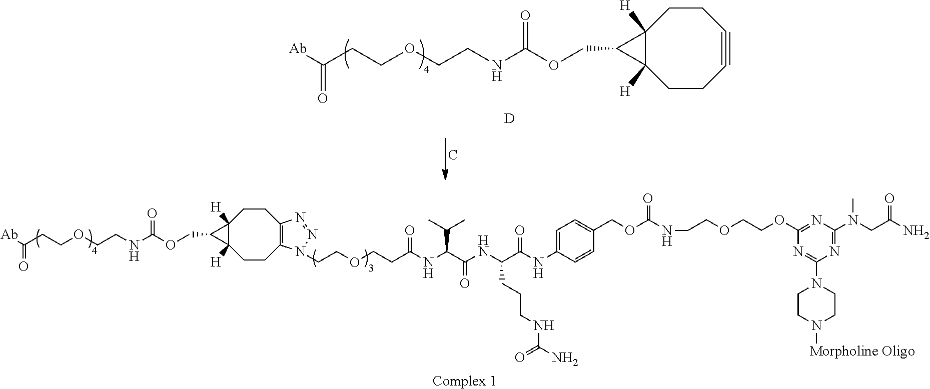

Compound D was stirred with 5-fold molar excess of Compound C in a glass bottle overnight at room temperature (˜22.5° C.) to provide Complex 1. Completion of the reaction was evaluated by SDS-PAGE and analytical SEC analysis, which demonstrated less than 10% unlinked anti-TfR antibody (DARO) and a 90% coupling efficiency by densitometry. The average crude DAR was found to be 1.7.

The crude complex pool was first purified via mixed-mode chromatography with ceramic hydroxyapatite type 1 resin. First, the ceramic hydroxyapatite (HA) column was pre-equilibrated with wash buffer (10 mM Na₂HPO₄, pH 5.7, 10% v/v IPA). After column equilibration, the crude reaction mixture was diluted 1:6 in 10 mM MES pH 5.7 containing 10 v/v % isopropanol to ensure a conductivity of less than 2 mS/cm. This material was loaded onto a ceramic hydroxyapatite (HA) column (HiLoad—50 mm×32 cm column, CHT™ 40 m resin from Biorad; Catalog #732-4324) at a protein concentration of 8 mg/mL of resin. For loading, the following parameters were used: linear flow rate of 214 cm/hr with 50 mm ID-70.0 ml/min volumetric flow, residence time-9 minutes]. Following loading, the HA column was washed with 5 CV of a wash solution (10 mM Na₂HPO₄, pH 5.7, 10% v/v IPA) to remove free oligonucleotide. The complex was subsequently eluted from the HA column with a step gradient to 100 mM Na₂HPO₄ pH 7.6 containing 10 v/v % isopropanol. Complete removal of free oligonucleotide was confirmed by analytical SEC. Yield post HA purification was determined to be 90% by BCA.

The purified complex buffer exchanged with a 30 kDa TFF membrane (Ultracel) into formulation buffer (25 mM Histidine, 10% w/v Sucrose, pH 6.0) using 10 diavolumes (targeting <20 TMP, Flux LMH 455 L/m²h, 200 mL/min). After diafiltration, the complex was concentrated to a target concentration of 7-10 mg/mL. Finally, the formulated complex was sterile filtered using PES Nalgene Rapidflow 0.2 um filters in a BSC and aliquoted into pre-autoclaved type 1 borosilicate glass vials. BCA analysis was used to measure protein concentration and calculate a final yield. DAR was calculated using densitometry results of SDS-PAGE. These data indicate a D0 of <10% and an average DAR at 1.93.

### Preparation of Complexes—Method 1

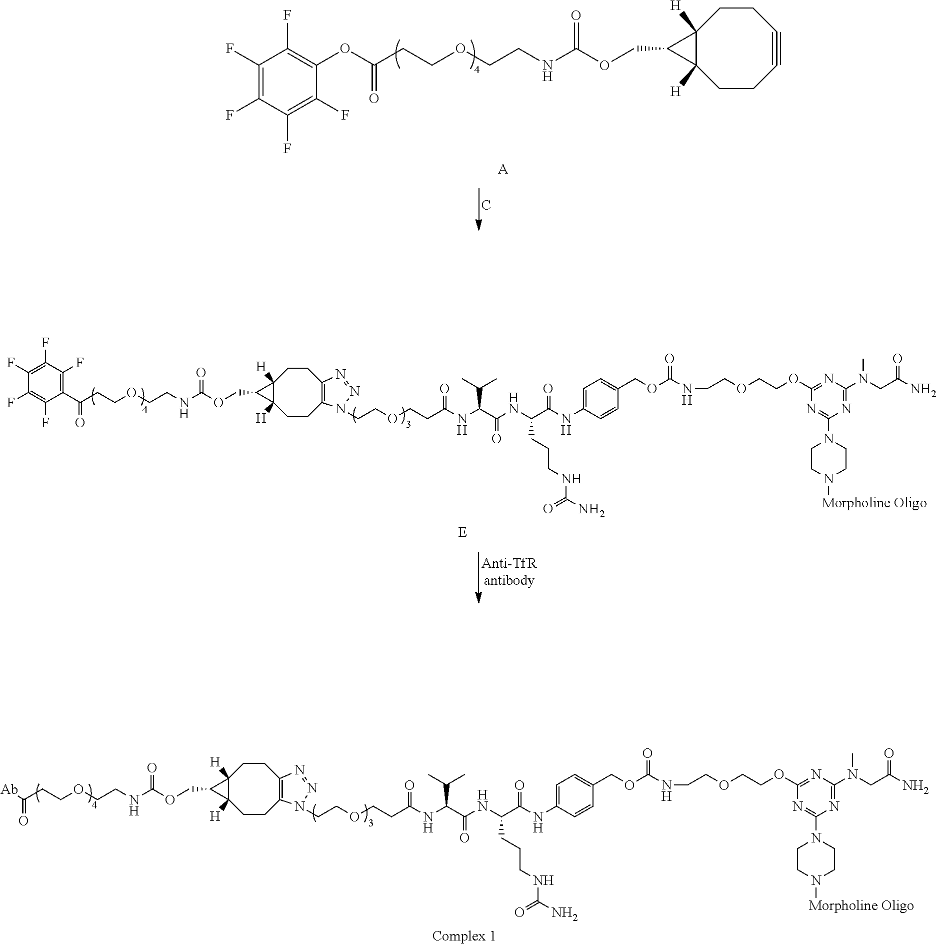

Lyophilized Compound C (98.1 mg) was solubilized in a 4 mL glass Wheaton vial in 0.32 mL of MilliQ water. Following solubilization, 0.32 mL of N,N-dimethylacetamide (DMA) was added and the mixture was gently agitated for 5-10 minutes. Prior to continuing, the vial was inspected carefully to ensure Compound C was completely dissolved and no residue remained on the walls of the glass vial. The concentration of the Compound C stock solution in 1:1 DMA:water was determined with a Nanodrop UV/vis instrument by using aliquots diluted 25-, 50-, and 100-fold in 1:1 DMA:water containing a final concentration of 0.1 M HCl at 265 nm, using an extinction coefficient of 318,050 M⁻¹cm¹. The HCl was added to ensure accuracy of the concentration measurement. The calculated concentration at each dilution was averaged to determine the solution concentration of 10.1 mM.

A 32.5 mg/mL (53.5 mM) stock solution of Compound A was prepared by weighing approximately 25 mg of Compound A into a 4 mL glass Wheaton vial. The appropriate volume of DMA was then added to afford the 32.5 mg/mL stock solution.

The reaction was conducted with the following final solution reaction conditions: 5.87 μM (6.5 μmol) Compound C and 5.34 mM Compound A (1.1:1.0 mol:mol equivalents) in 60:40 v/v % DMA to 25 mM 2-(N-morpholino)ethanesulfonic acid (MES) pH 5.5 buffer at room temperature. The reaction was set-up in a 4 mL glass Wheaton vial by adding the appropriate amounts of the reactants and stock solutions as indicated in Table 4. The total final volume of the reaction was 1.11 mL.

TABLE 4

| Addition | | Stock | Volume |
|---|---|---|---|
| Order | Solution | Concentration | (mL) |
| 1 | MES, pH 5.5 buffer stock | 500 mM | 0.0222 |
| 2 | MilliQ Water | NA | 0.0981 |
| 3 | DMA | NA | 0.2313 |
| 4 | Compound C stock in 1:1 | 10.1 mM | 0.6456 |
| | DMA:MilliQ water | | |
| 5 | Compound A stock in DMA | 53.5 mM | 0.1105 |

Completion of the reaction to generate Compound E was monitored by RP-C18 UPLC with repeat injections every 30 minutes (at 5 minute, 35 minute, and 65 minute time points), by observing the disappearance of Compound A starting material at 220 nm. This IPC indicated the reaction was complete at 65 minutes. The reaction was determined to be complete when less than 5% of Compound A was remaining. The crude reaction mixture was carried forward to the conjugation reaction immediately without any purification. The total reaction time was 90 minutes defined as the time between addition of Compound A to the reaction and addition of this reaction mixture to the Fab′ conjugation reaction.

Conjugation of Compound E to the anti-TfR Fab′ involves the formation of an amide bond between solvent accessible lysine residues of the Fab′ and the activated ester of Compound E.

Prior to setup of the conjugation reaction, the anti-TfR Fab′ formulated in 20 mM sodium citrate, 100 mM sodium chloride was buffer exchanged into 50 mM HEPES pH 7.5. Anti-TfR Fab′ (10 mL at 10.15 mg/mL) was loaded onto 50 mM HEPES pH 7.5 equilibrated NAP-25 desalting columns (4×2.5 mL) and eluted with 50 mM HEPES pH 7.5 (4×3.5 mL). The eluate was pooled and concentrated with an Amicon Ultra-15 10 kDa centrifugal filter unit spinning at 4000 rcf to reduce the volume to 2.86 mL. The concentration of the buffer resultant anti-TfR Fab′ was measured by Nanodrop UV/vis to be 31.75 mg/mL.

To provide Complex 1, the conjugation reaction was conducted with the following final solution reactant amounts: anti-TfR Fab′ (45 mg, 6 mg/mL, 125 μM) and Compound E at a final theoretical concentration of 750 μM (6.0:1.0 mol:mol equivalents of Compound E vs anti-TfR Fab′). Compound E concentration assumed 100% conversion in the first reaction. The final reaction mixture consisted of 15:85 v/v % DMA to 25 mM HEPES pH 7.5 buffer. The reaction was set-up in a 20 mL glass scintillation vial by adding the appropriate amounts of the reactants and stock solutions as indicated in Table 5 The reaction proceeded for 20 h at room temperature (˜25° C.). The start of the reaction was defined as the addition time of the pre-reaction mixture containing Compound E to the anti-TfR Fab′ solution. The total duration of the conjugation reaction was 20 hours.

TABLE 5

| Addition | | Stock | | Volume |
|---|---|---|---|---|
| Order | Solution | Concentration | | (mL) |
| 1 | HEPES, pH 7.5 buffer stock | 500 | mM | 0.177 |
| 2 | MilliQ Water | — | | 4.360 |
| 3 | DMA | — | | 0.492 |
| 4 | Anti-TfR Fab′ (31.75 mg/mL | 662 | μM | 1.417 |
| | in 50 mM HEPES, pH 7.5) | | | |
| 5 | Compound E | 5.34 | mM | 1.055 |

After reacting for 20 hours, the crude complex mixture was tested by SDS-PAGE and analyzed by densitometry to determine the drug to antibody ratio (DAR) and % unconjugated Fab′. Results are presented in Table 6.

TABLE 6

| Species | Abundance |
|---|---|
| D0 | 0.090 |
| D1 | 0.290 |
| D2 | 0.333 |
| D3 | 0.200 |
| D4 | 0.061 |
| D5 | 0.020 |
| D6 | 0.006 |
| Average DAR | 1.93 |

### Purification of Complex 1

Following synthesis of Complex 1 by Method 1 described above, a two-part purification process was conducted. First, free payload was removed by hydroxyapatite (HA) chromatography. The HA eluate was then buffer exchanged into the final formulation. At the 45 mg scale of Fab′, the final buffer exchange was performed with a 30 kDa centrifugal filter device. Prior to loading onto the HA column, the crude reaction product from Method 1 (anti-TfR Fab′-oligonucleotide complex) was diluted and the pH adjusted from 7.5 to 5.7. First, the 7.5 mL of crude complex was diluted by addition of 16.5 mL of 15 v/v % DMA in water and the solution was thoroughly mixed. To this mixture, 0.75 mL of 500 mM MES (pH 3.3) was added to adjust the pH down to 5.7.

Chromatographic purification to remove unreacted oligonucleotide species was performed using a 5 mL Bio Rad CHT Type I (ceramic hydroxyapatite) cartridge on an AKTA Pure chromatography system. Prior to loading the diluted complex pool from the reaction mixture preparation step, the CHT cartridge was prepared and equilibrated according to the manufacturer's instructions using 15:85 v/v % of DMA to 10 mM sodium phosphate, pH 5.8 buffer. Following equilibration, the complex pool was loaded at a flow rate of 5 mL/min. After loading the complex, the column was washed for a minimum of 7 CV with 15:85 v/v % of DMA in 10 mM sodium phosphate buffer (pH 5.8). After completion of the wash, elution was initiated via a step gradient with 100 mM sodium phosphate, pH 7.6 buffer containing DMA at 15:85 v/v % at a flow rate of 5 mL/min. The entire elution peak, identified by monitoring at 260 nm and 280 nm, was collected and pooled.

Analysis of the flow through during the HA column loading step by SEC indicated the presence of little to no Fab′-oligonucleotide complex in the flow-through. Only peaks due to oligonucleotide payload species were observed, at ˜10.5 and ˜11.3 minutes. Conversely, SEC analysis of the pooled elution peak showed only complex species, with multiple peaks and shoulders due to the size differences of the complexes with different oligonucleotide (e.g., PMO) payload loadings. No peaks for payload species at 10.5 or 11.3 minutes were observed. The complex mass balance with respect to Fab′ for the HA purification was estimated by SEC chromatography. This was accomplished by injecting 24 ug of Fab′ from the crude conjugation reaction product (4 μL injection at 6 μg/mL concentration) and injecting a theoretical 24 μg of Fab′, assuming 100% recovery, from HA eluate pool. The HA eluate pool was 13.9 mL theoretically containing 45 mg of Fab′, giving a theoretical concentration of 3.24 μg/p L. To achieve an injection of 24 μg of Fab′, a 7.4 μL injection was used. SEC indicated a 97% recovery of Complex 1 after the HA chromatographic purification.

At the 45 mg reaction scale, buffer exchange of the HA eluate into 50 mM His (pH 6.0) was performed using an Amicon Ultra-15 30 kDa centrifugal filtration device. First, the HA eluate pool was concentrated to approximately 1.5 mL by spinning the column at 4000 rcf. Buffer exchange was subsequently performed via addition of 3 mL of 50 mM His (pH 6.0) and concentration by centrifugation at 4000 rcf until the volume reached approximately 1.5 mL. This step was repeated for five total rounds, using the equivalent of 15 volumes of buffer to generate the final purified Complex 1. The resulting ˜1.5 mL of purified complex (in 50 mM His, pH 6.0) was then diluted to a final volume of 3.0 mL with additional 50 mM His (pH 6.0).

The final purified Complex 1 was analyzed by SEC, SDS-PAGE densitometry, and BCA. SEC of the final complex was nearly identical to the corresponding SEC data of the complex pool after HA purification, indicating the purification process did not induce formation of high molecular weight species. The average DAR, DAR species distribution, and percent unconjugated Fab′ were calculated by SDS-PAGE densitometry (SDS-PAGE gel) and data analysis was performed with the Image Studio Lite software package from Li-Cor Biosciences (calculation results shown in Table 7). The final average DAR of the purified Complex 1 was 1.96, including 8.1% unconjugated anti-TfR Fab′. Protein concentration was measured to be 10.5 mg/mL by the BCA assay, indicating a total of 31.4 mg of complex in the final product, for an overall process yield of 70%.

TABLE 7
Average DAR and DAR distribution of the purified Complex 1.

| Species | Abundance |
|---|---|
| D0 | 0.081 |
| D1 | 0.324 |
| D2 | 0.312 |
| D3 | 0.168 |
| D4 | 0.071 |
| D5 | 0.032 |
| D6 | 0.011 |
| Average DAR | 1.96 |

### Effect of the Ratio of Compound E to Fab′ and Reaction Conditions on Conjugation

To investigate the effect of the ratio of Compound E from the reaction mixture to anti-TfR, as well as the effect of oligonucleotide and Fab′ concentrations in oligonucleotide-Fab′ conjugation reactions, a set of reactions were conducted according to the general protocol described for Method 1 above. The oligonucleotide used was a PMO of 30 nucleotides in length.

In a first set of reactions, the reaction between Compound A and Compound C was conducted using the following conditions: 2.44 mM Compound C with Compound A (1.5:1 mol:mol ratio) were reacted in a 1:1 mixture of DMA and 25 mM MES pH 5.5 buffer. The total reaction volume was 0.37 mL, and the reaction step was allowed to proceed for 18 h at room temperature (˜25° C.).

Upon completion of the reaction, the crude (i.e., non-purified) reaction mixture containing Compound E was used to set-up a series of anti-TfR Fab′ conjugation reactions. The anti-TfR Fab′ conjugation reactions were conducted using 2, 4, 6, and 10 molar equivalents of Compound E with respect to Fab′. All other reaction conditions were held constant across the different conjugations. In the conjugation reaction mixture, the Fab′ concentration was 3 mg/mL with 1 mg total Fab′ per reaction in 15 v/v % DMA in 50 mM HEPES pH 7.5 buffer. The conjugation reaction was conducted at 23-25° C. for 18 h. The final DAR and DAR species distributions for each complex were determined by SDS-PAGE densitometry and the results are shown in Table 8.

TABLE 8

| | | Peak Fraction | | | | | | | |
|---|---|---|---|---|---|---|---|---|---|
| Reaction | Compound E equiv. | D0 | D1 | D2 | D3 | D4 | D5 | D6 | Avg. DAR |
| A | 2 | 0.389 | 0.412 | 0.159 | 0.040 | ND | ND | ND | 0.85 |
| B | 4 | 0.232 | 0.391 | 0.256 | 0.095 | 0.027 | ND | ND | 1.30 |
| C | 6 | 0.150 | 0.361 | 0.308 | 0.117 | 0.053 | 0.012 | ND | 1.60 |
| D | 10 | 0.084 | 0.293 | 0.342 | 0.137 | 0.077 | 0.044 | 0.024 | 2.06 |

In a second set of reactions, the reaction between Compound A and Compound C was conducted using the following conditions: 6.77 mM Compound C with 4.84 mM Compound A (1.4:1 mol:mol ratio) were reacted in a 1:1 mixture of DMA and 25 mM MES pH 5.5 buffer. The total reaction volume was 0.160 mL, and the reaction was allowed to proceed for 90 minutes at room temperature (˜25° C.).

Upon completion of the reaction, the crude (i.e., non-purified) pre-reaction mixture containing Compound E was used to set-up a series of anti-TfR Fab′ conjugation reactions. The anti-TfR Fab′ conjugation reactions were conducted using 2, 4, 5, 6, and 8 molar equivalents of Compound E with respect to Fab′. All other reaction conditions were held constant across the different conjugations. In the conjugation reaction mixture, the Fab′ concentration was 6 mg/mL with 1 mg total Fab′ per reaction in 15 v/v % DMA in 50 mM HEPES pH 7.5 buffer. The conjugation reaction was conducted at 23-25° C. for 19 h. The final average DAR for each complex was determined by SDS-PAGE densitometry and the results are shown in Table 9.

TABLE 9

| | Compound | Avg. |
|---|---|---|
| Reaction | E equiv. | DAR |
| E | 2 | 1.14 |
| F | 4 | 1.64 |
| G | 5 | 1.93 |
| H | 6 | 2.06 |
| I | 8 | 2.21 |

In a third set of reactions, the reaction between Compound A and Compound C was conducted using the following conditions: 6.77 mM Compound C with 4.84 mM Compound A (1.4:1 mol:mol ratio) were reacted in a 60:40 mixture of DMA and 25 mM MES pH 5.5 buffer. The total reaction volume was 0.160 mL, and the reaction was allowed to proceed for 90 minutes at room temperature (˜25° C.).

Upon completion of the reaction, the crude (i.e., non-purified) pre-reaction mixture containing Compound E was used to set-up a series of anti-TfR Fab′ conjugation reactions. The anti-TfR Fab′ conjugation reactions were conducted using 2, 4, 5, 6, 8, and 10 molar equivalents of Compound E with respect to Fab′. All other reaction conditions were held constant across the different conjugations. In the conjugation reaction mixture, the Fab′ concentration was 6 mg/mL with 1 mg total Fab′ per reaction in 15 v/v % DMA in 50 mM HEPES pH 7.5 buffer. The conjugation reaction was conducted at room temperature (˜25° C.) for 19 h. The final average DAR and percentage of DARO for each complex was determined by SDS-PAGE densitometry and the results are shown in Table 10 (reactions J, K, L, M, N, O).

In a fourth set of reactions, the reaction between Compound A and Compound C was conducted using the following conditions: 6.77 mM Compound C with Compound A (1.05:1 mol:mol ratio) were reacted in a 60:40 mixture of DMA and 25 mM MES pH 5.5 buffer. The total reaction volume was 0.080 mL, and the reaction was allowed to proceed for 160 minutes at room temperature (˜25° C.).

Upon completion of the reaction, the crude (i.e., non-purified) reaction mixture containing Compound E was used to set-up a series of anti-TfR Fab′ conjugation reactions. The anti-TfR Fab′ conjugation reactions were conducted using 5, 6, and 7 molar equivalents of Compound E with respect to Fab′. All other reaction conditions were held constant across the different conjugations. In the conjugation reaction mixtures, the Fab′ concentration was 6 mg/mL or 12 mg/mL with 0.6 mg and 1.2 mg total Fab′ per reaction, respectively. All reactions were conducted in 15 v/v % DMA in 25 mM HEPES pH 7.5 buffer. The conjugation reactions were conducted at room temperature (˜25° C.) for approximately 18 h. The final average DAR and percentage of DARO for each complex was determined by SDS-PAGE densitometry and the results are shown in Table 10 (reactions P, Q, R, S).

TABLE 10

| | Compound | Fab′ | Avg. | % |
|---|---|---|---|---|
| Reaction | E equiv. | (mg/mL) | DAR | D0 |
| J | 2 | 6 | 1.11 | 28.4 |
| K | 4 | 6 | 1.62 | 18.1 |
| L | 5 | 6 | 1.90 | 13.4 |
| M | 6 | 6 | 2.41 | 11.2 |
| N | 8 | 6 | 2.57 | 8.7 |
| O | 10 | 6 | 3.34 | 4.6 |
| P | 5 | 6 | 2.01 | 12.1 |
| Q | 6 | 6 | 2.17 | 11.8 |
| R | 7 | 6 | 2.34 | 9.0 |
| S | 6 | 12 | 2.30 | 9.3 |

### Alternative Preparation of Complex 1 by Method 1—Purification of Compound E

Lyophilized compound C (200 mg) was solubilized in a 20 mL glass Wheaton vial targeting a theoretical concentration of approximately 30 mg/mL by addition of 7 mL of DMSO. A clear solution was obtained after gentle agitation for 5 minutes. The concentration of the compound C stock solution in DMSO was determined with a Nanodrop UV/vis instrument by using aliquots diluted 50-fold in 0.1 M HCl at 265 nm using an extinction coefficient of 318050 M⁻¹cm⁻¹. The calculated concentration was 24 mg/mL (2.3 mM).

A 30 mg/mL (49.4 mM) stock solution of compound A was prepared by weighing approximately 50 mg of the compound A ester oil into a 4 mL glass vial. 1.6 mL of DMA was then added to afford the 30 mg/mL stock solution. No UV/vis assay or purity correction was performed.

2.02 mM (168 mg; 7 mL of the stock solution prepared above) compound C and 6.06 mM of compound A (1.3:1.0 mol:mol equivalents; 0.98 mL of the stock solution prepared above) were combined at room temperature. The reaction was set up in a 20 mL glass vial.

Completion of the reaction to generate compound E was monitored by RP-C18 (Cortex T3 column) UPLC with repeat injections every 30 minutes observing the peak shift rate from compound C to compound E at 260 nm. The reaction was determined to be complete when the peak shift rate no longer changed after 2 hours at room temperature. The crude reaction mixture was purified by adding 64 mL of ice-cold acetone (8:1 vol/vol equivalents of reaction volume). The tube was then centrifuged at 4500 rpm for 20 minutes and the supernatant was discarded. The pellet was washed with acetonitrile (room temperature) three times. After the acetonitrile was removed, 8 mL of 20% acetonitrile in 20 mM MES buffer (pH 5.0) was added to the tube to reconstitute the pellet. The final yield of compound E was 160 mg (95%). Similar results were observed when 64 mL of ice-cold IPA was used for the purification of compound E.

Anti-TfR Fab formulated in 20 mM sodium citrate and 100 mM sodium chloride was buffer exchanged into 50 mM EPPS at pH 8.0. Anti-TfR Fab (100 mL at 10.15 mg/mL) was buffer exchanged using 50 cm² TFF membrane (10 kDa MWCO; Satorius PES) with 7 diavolumes of 50 mM EPPS pH 8.0. The retentate pool was then concentrated to ˜40 mL, targeting a concentration of ˜25 mg/mL.

The conjugation reaction was conducted with the following final solution reactant parameters: anti-TfR Fab (5 mg at 3 mg/mL, 62.5 uM) and compound E at a final theoretical concentration of 187.5 uM/250 uM/312.5 uM/375 uM (1:3/1:4/1:5/1:6 mol:mol equivalents of anti-TfR Fab to compound E). Compound E concentration assumes 75% conversion of compound C into compound E. The final reaction mixture comprises 15:85 v/v % DMA to 50 mM EPPS pH 8.0 buffer. The reaction was set up in a 15 mL falcon tube by adding the appropriate amounts of the reactants and stock solutions as indicated in Table 11 and was allowed to stand for 18 h at room temperature

TABLE 11

| Addition | | Stock | | |
|---|---|---|---|---|
| Order | Solution | Concentration | | Volume (mL) |
| 1 | EPPS, pH 8.0 buffer stock | 500 | mM | 0.25 |
| 2 | MilliQ Water | — | | 0.894-0.821 |
| 3 | DMA | — | | 0.25 |
| 4 | anti-TfR Fab (25 mg/mL in | 521 | uM | 0.2 |
| | 50 mM EPPS, pH 8.0) | | | |
| 5 | Compound E | 1.92 | mM | 0.073(3×), |
| | | | | 0.098(4×), |
| | | | | 0.122(5×), |
| | | | | 0.146(6×) |

After reacting overnight or 18 ours, the crude conjugate mixture was tested by SDS-PAGE and analyzed by densitometry to determine the DAR and % unconjugated anti-TfR Fab (Table 12).

TABLE 12

| Compound E | | Percentage |
|---|---|---|
| molar excess | Average DAR | DAR0 |
| 3 | 2.58 | 4 |
| 4 | 2.91 | 0 |
| 5 | 3.14 | 0 |
| 6 | 3.49 | 0 |

5 mg of Complex 1 made with 4 excesses of compound E (DAR 2.91 without DO) was purified by HA chromatography, and the HA eluent was buffer exchanged into the final formulation (25 mM His, 10% w/v Sucrose, pH 6.0).

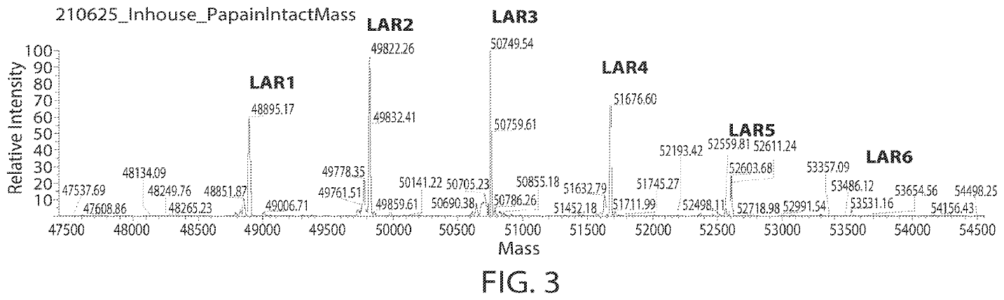

Complex 1 for LC-MS analysis was prepared by removing the payload through papain digestion. Immobilized papain (Thermo 20341) was activated in papain buffer (20 mM sodium phosphate, 10 mM EDTA, 20 mM cysteine, pH 7.0) for 15 minutes prior to digestion. Conjugate samples were buffer exchanged into digestion buffer (20 mM sodium phosphate, 10 mM EDTA pH 7.0) using a Thermo Zeba 7 MWCO spin filter. 40 μL of protein sample was added to 100 μL of a 50% immobilized papain slurry. The samples were incubated at 40° C. for 1 hour with shaking at 1400 rpm. After incubation, the digests were separated from the resin through spin filtration. Complete payload removal was confirmed prior to LC-MS analysis by gel electrophoresis. Samples were again buffer exchanged prior to LC-MS into Optima grade water. 1 μg of total protein was injected on the Thermo Q-Exactive LC-MS for routine intact mass analysis. The resulting mass spectrum is shown in FIG. 3, demonstrating the extent of conjugation of linker to antibody (linker-to-antibody ratio, LAR).

### Preparation of Compound E-1

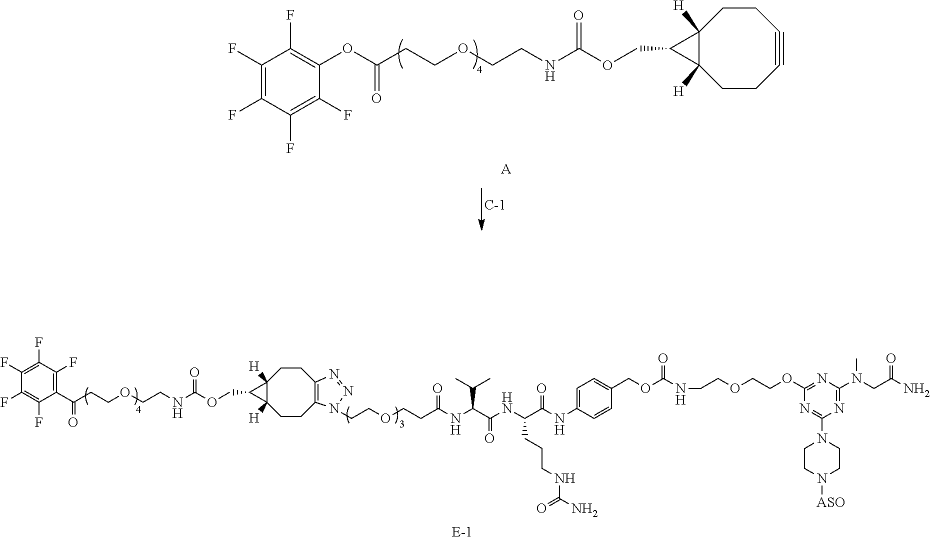

A 20 mg/mL (32.92 mM) stock solution of compound A was prepared in DMA. 2.5 mM (198 mg; 5.4 mL of a 6.5 mM stock solution in water with 15% acetonitrile) compound C-1, 7.5 mM compound A (1.0:3.0 mol:mol equivalents; 3.2 mL of the stock solution prepared above), and 0.14 mL 10 mM MES buffer (pH 5.0) were combined in 5.2 mL DMA at room temperature. The reaction was set-up in a 20 mL glass vial. The reaction was carried out at room temperature for 90 minutes. Completion of the reaction was monitored by RP-C18 UPLC and LC-MS methods. Once the reaction was determined to be complete, 1.4 mL of a 3M NaCl solution (1/10 volume of crude reaction mixture) and 42 mL of ice-cold IPA (3 volumes of crude reaction mixture) were added to the mixture and the tube was cooled to −80° C. for more than 30 minutes. The tube was then centrifuged at 4500 rpm for 20 minutes and the supernatant was discarded. The pellet was washed with 75% ethanol (room temperature) twice. After all traces of ethanol were removed, 5 mL of 15% acetonitrile in 25 mM MES buffer (pH 5.0) was added to the tube to reconstitute the pellet. 196 mg (99%) compound E-1 was isolated.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

For reasons of completeness, various aspects of the present disclosure are set out in the following numbered clauses:

1. A method of preparing a compound of Formula (I):

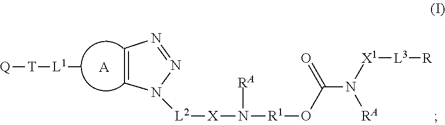

or a salt thereof, the method comprising coupling a targeting agent (Q) with a compound of Formula (II):

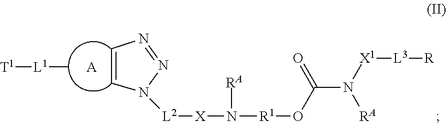

or a salt thereof, to provide a compound of Formula (I), wherein:
T is

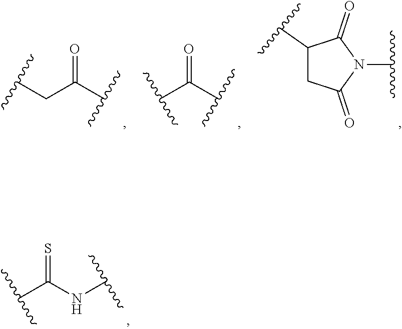

or —S—;
T¹ is

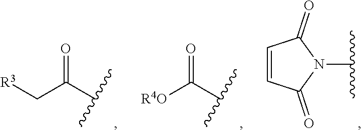

S═C═N—, or

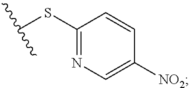 [SMILES: *Sc1ccc([N+](=O)[O-])cn1]

R³ is a leaving group (e.g., halogen, tosylate, mesylate, or triflate);
R⁴ is hydrogen; substituted or unsubstituted alkyl; substituted or unsubstituted heterocyclyl; substituted or unsubstituted aryl; substituted or unsubstituted heteroaryl; or an oxygen protecting group;
L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
A is substituted or unsubstituted carbocycle or substituted or unsubstituted heterocycle, or A is absent and L¹ is bonded directly to the triazole ring;
L² is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
X is a cleavable moiety;
R¹ is substituted or unsubstituted arylene, or substituted or unsubstituted alkylene, or a combination thereof;
X¹ is a bond or a peptide;
L³ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
each R^{A} is independently hydrogen or substituted or unsubstituted alkyl; and
R is a molecular payload.
   1a. The method of clause 1, wherein the coupling of the targeting agent (Q) with a compound of Formula (II) takes place in solvent comprising dimethyl acetamide or isopropyl alcohol.
   1b. The method of clause 1 or 1a, wherein the coupling of a targeting agent (Q) with a compound of Formula (II) is performed at a pH of about 7.0 to about 8.5, about 7.0 to about 8.0, about 7.5 to about 8.5, or about 7.5 to about 8.0.
   1c. The method of clause 1 or 1a, wherein the coupling of a targeting agent (Q) with a compound of Formula (II) is performed at a pH of about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, or about 8.5.
   1d. The method of any of clauses 1 or 1a-1c, wherein the coupling of a targeting agent (Q) with a compound of Formula (II) further comprises adding a buffer having a pKa of about 7.0 to about 8.5, about 7.0 to about 8.0, about 7.5 to about 8.5, or about 7.5 to about 8.0.
   1e. The method of any of clauses 1 or 1a-1c, wherein the coupling of a targeting agent (Q) with a compound of Formula (II) further comprises adding a buffer having a pKa of about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, or about 8.5.
   1f. The method of clauses 1d or 1e, wherein the buffer is HEPES (N-(2-Hydroxyethyl)piperazine-N′-(2-ethanesulfonic acid)) or EPPS (N-(2-Hydroxyethyl)piperazine-N′-(3-propanesulfonic acid)).
   2. The method of clause 1, further comprising reacting a compound of Formula (III):

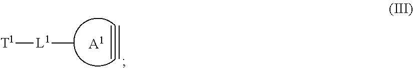

or a salt thereof, with a compound of Formula (IV):

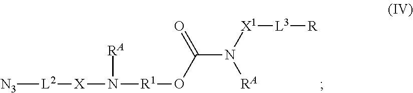

or a salt thereof, to provide a compound of Formula (II), or a salt thereof, wherein:
A¹ is substituted or unsubstituted carbocycle or substituted or unsubstituted heterocycle, or A is absent and L¹ is bonded directly to one terminus of the alkyne.
   2a. The method of clause 2, wherein the reacting of a compound of Formula (III) with a compound of Formula (IV) takes place in solvent comprising dimethyl sulfoxide, acetonitrile, water, dimethyl acetamide, or isopropyl alcohol.
   2b. The method of clause 2, the method further comprising purifying the compound of Formula (II).
   2c. The method of clause 2b, wherein purifying the compound of Formula (II) comprises adding, to the reaction mixture comprising the completed reaction of the compound of Formula (III) with the compound of Formula (IV), a volume of a solvent that is about 2, 3, 4, 5, 6, 7, 8, 9, or 10 times the amount of the total volume of the reaction mixture of the compound of Formula (III) with the compound of Formula (IV); and isolating the compound of Formula (II) from the reaction mixture.
   2d. The method of clause 2b or 2c, wherein the volume of solvent is about 8 times the amount of the total volume of the reaction mixture of the compound of Formula (III) with the compound of Formula (IV).
   2e. The method of clause 2b or 2c, wherein the volume of solvent is about 3 times the amount of the total volume of the reaction mixture of the compound of Formula (III) with the compound of Formula (IV).
   2f. The method of any of clauses 2c-2e, wherein the solvent is acetone or isopropyl alcohol.
   2g. The method of any of clauses 2c-2f, wherein the solvent added in the purification is at a temperature that is equal to or below room temperature, 30° C., 25° C., 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C.
   2h. The method of any of clauses 2c-2f, wherein the solvent added in the purification is at a temperature of about 0° C.
   2i. The method of any of clauses 2b-2h, wherein purifying the compound of Formula (II) further comprises adding, to the reaction mixture comprising the completed reaction of the compound of Formula (III) with the compound of Formula (IV), a volume of an aqueous solution of a salt that is about 1, 0.9, 0.8, 0.7, 0.6, 0.5, 0.4, 0.3, 0.2, or 0.1 times the amount of the total volume of the reaction mixture of the compound of Formula (III) with the compound of Formula (IV).
   2j. The method of any of clauses 2b-2i, wherein purifying the compound of Formula (II) further comprises adding, to the reaction mixture comprising the completed reaction of the compound of Formula (III) with the compound of Formula (IV), a volume of a 3M aqueous solution of a salt that is about 0.1 times the amount of the total volume of the reaction mixture of the compound of Formula (III) with the compound of Formula (IV).
   2k. The method of clause 2i or 2j, wherein the salt is NaCl.
   2l. The method of any of clauses 2b-2k, wherein purifying the compound of Formula (II) further comprises cooling the reaction mixture to a temperature that is equal to or below 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C.
   3. The method of clause 1 or 2, further comprising reacting a compound of Formula (V):

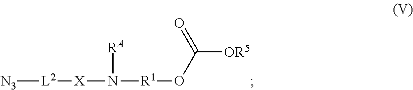

or a salt thereof, with a compound of Formula (VI):

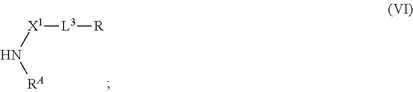

or a salt thereof, to provide a compound of Formula (IV), or a salt thereof, wherein:
R⁵ is substituted or unsubstituted aryl, or substituted or unsubstituted heteroaryl.
   4. The method of any of clauses 1-3, further comprising reacting a compound of Formula (VII):

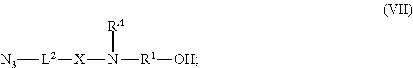

or a salt thereof, with a compound of Formula (VIII):

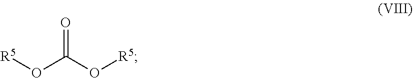

or a salt thereof, to provide a compound of Formula (V), or a salt thereof, wherein:
R⁵ is substituted or unsubstituted aryl, or substituted or unsubstituted heteroaryl.
   5. The method of any of clauses 1-4, further comprising reacting a compound of Formula (IX):

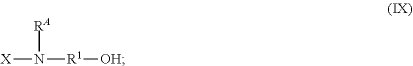

or a salt thereof, with a compound of Formula (X):

N₃-L²-LG (X);

or a salt thereof, to provide a compound of Formula (VII), or a salt thereof, wherein:
LG is a leaving group.
   6. The method of clause 1, wherein the compound of Formula (I) is of Formula (I-c):

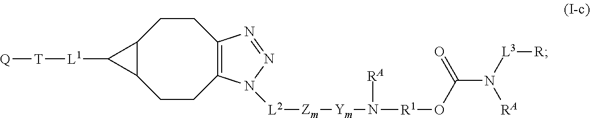

or a salt thereof, and the compound of Formula (II) is of Formula (II-c):

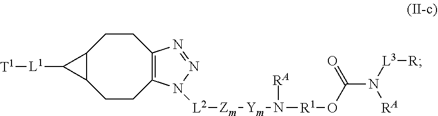

or a salt thereof, wherein:
each occurrence of Z is independently an amino acid;
each occurrence of Y is independently an amino acid; and
each m is independently 1, 2, or 3.
   7. The method of clause 6, further comprising reacting a compound of Formula (III-a):

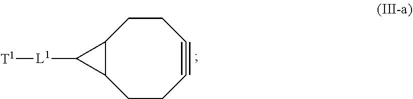

or a salt thereof, with a compound of Formula (IV-b):

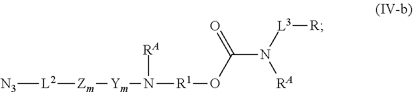

or a salt thereof, to provide a compound of Formula (II-c), or a salt thereof.
8. The method of clause 6 or 7, further comprising reacting a compound of Formula (V-a):

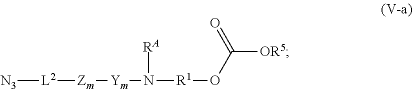

or a salt thereof, with a compound of Formula (VI-a):

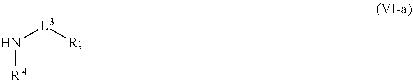

or a salt thereof, to provide a compound of Formula (IV-b), wherein:
R⁵ is substituted or unsubstituted aryl, or substituted or unsubstituted heteroaryl.
   9. The method of any of clauses 6-8, further comprising reacting a compound of Formula (VII-a):

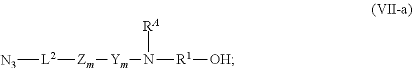

or a salt thereof, with a compound of Formula (VIII):

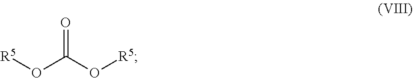

or a salt thereof, to provide a compound of Formula (V-a), wherein:
R⁵ is substituted or unsubstituted aryl, or substituted or unsubstituted heteroaryl.
   10. The method of any of clauses 6-9, further comprising reacting a compound of Formula (IX-a):

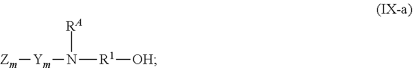

or a salt thereof, with a compound of Formula (X):

N₃-L²-LG (X);

or a salt thereof, to provide a compound of Formula (VII-a), wherein:
LG is a leaving group and is displaced by the amino group of the terminal amino acid Z.
   11. The method of clause 1 or 6, wherein the compound of Formula (I) is of Formula (I-e):

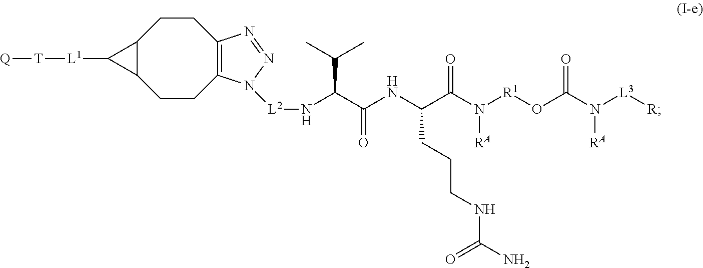

or a salt thereof, and the compound of Formula (II) is of Formula (II-e):

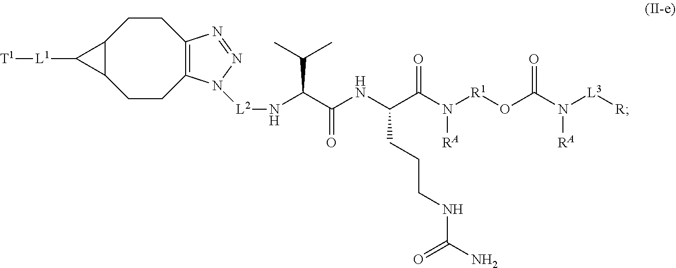

or a salt thereof.
12. The method of clause 11, further comprising reacting a compound of Formula (III-a):

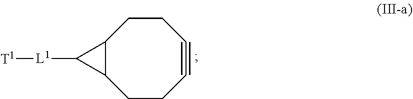

or a salt thereof, with a compound of Formula (IV-d):

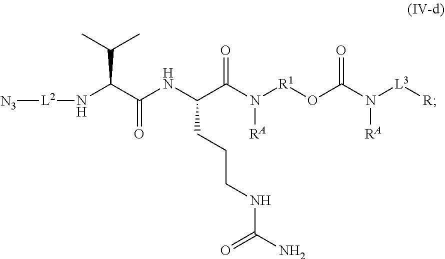

or a salt thereof, to provide a compound of Formula (II-e), or a salt thereof.
13. The method of clause 11 or 12, further comprising reacting a compound of Formula (V-c):

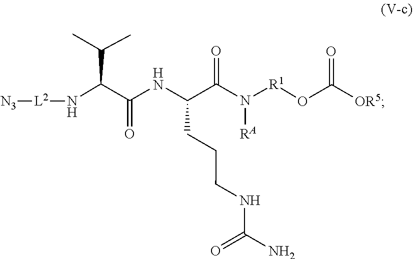

or a salt thereof, with a compound of Formula (VI-a):

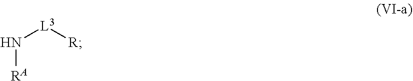

or a salt thereof, to provide a compound of Formula (IV-d), wherein:
R⁵ is substituted or unsubstituted aryl, or substituted or unsubstituted heteroaryl.
   14. The method of any of clauses 11-13, further comprising reacting a compound of Formula (VII-c):

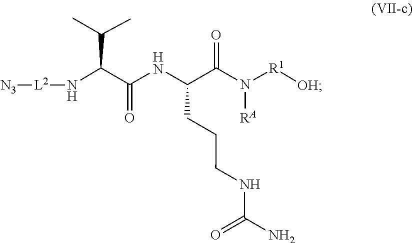

or a salt thereof, with a compound of Formula (VIII):

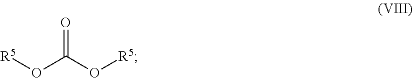

or a salt thereof, to provide a compound of Formula (V-c), wherein:
R⁵ is substituted or unsubstituted aryl, or substituted or unsubstituted heteroaryl.
   15. The method of any of clauses 11-14, further comprising reacting a compound of Formula (IX-c):

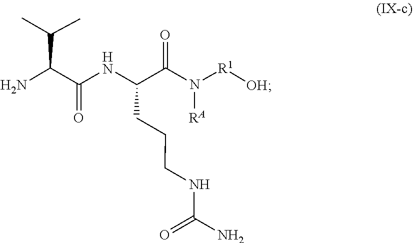

or a salt thereof, with a compound of Formula (X):

N₃-L²-LG (X);

or a salt thereof, to provide a compound of Formula (VII-c), wherein:
LG is a leaving group.
   16. The method of any of clauses 1, 6, or 11, wherein the compound of Formula (I) is of Formula (I-f):

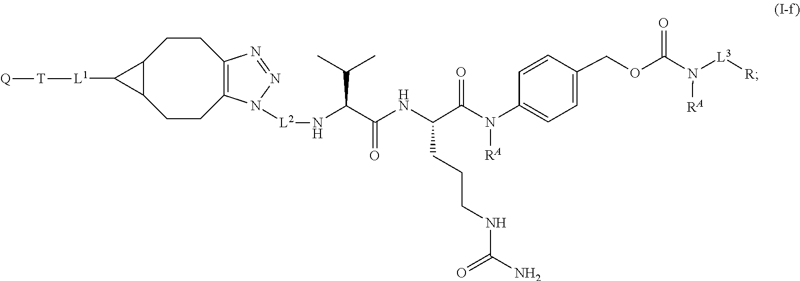

or a salt thereof, and the compound of Formula (II) is of Formula (IIf):

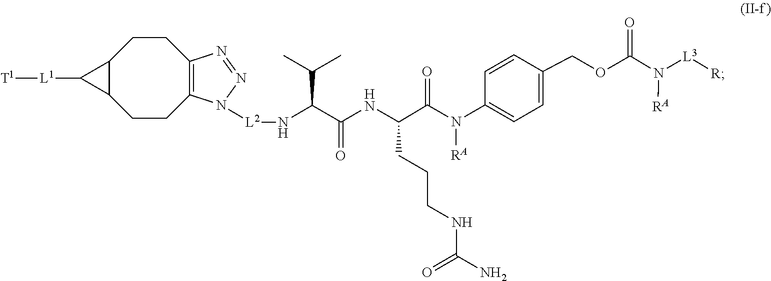

or a salt thereof.
17. The method of clause 11, further comprising reacting a compound of Formula (III-a):

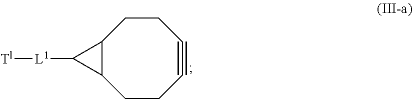

or a salt thereof, with a compound of Formula (IV-e):

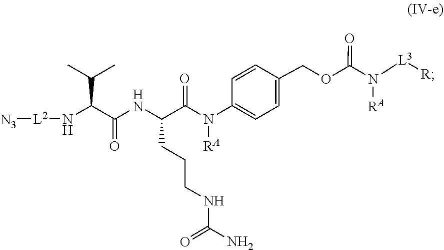

or a salt thereof, to provide a compound of Formula (II-f), or a salt thereof.
18. The method of clause 16 or 17, further comprising reacting a compound of Formula (V-d):

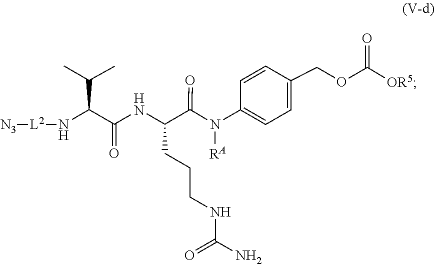

or a salt thereof, with a compound of Formula (VI-a):

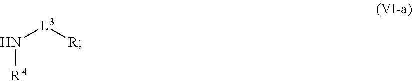

or a salt thereof, to provide a compound of Formula (IV-e), wherein:
R⁵ is substituted or unsubstituted aryl, or substituted or unsubstituted heteroaryl.
   19. The method of any of clauses 16-18, further comprising reacting a compound of Formula (VII-d):

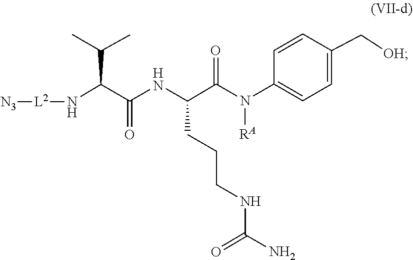

or a salt thereof, with a compound of Formula (VIII):

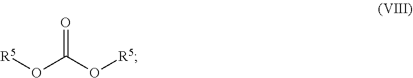

or a salt thereof, to provide a compound of Formula (V-d), wherein:
R⁵ is substituted or unsubstituted aryl, or substituted or unsubstituted heteroaryl.
   20. The method of any of clauses 16-19, further comprising reacting a compound of Formula (IX-d):

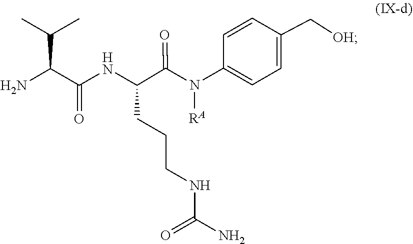

or a salt thereof, with a compound of Formula (X):

N₃-L²-LG (X);

or a salt thereof, to provide a compound of Formula (VII-d), wherein:
LG is a leaving group.
   21. The method of any of clauses 1, 6, 11, or 16, wherein the compound of Formula (I) is of Formula (I-g):

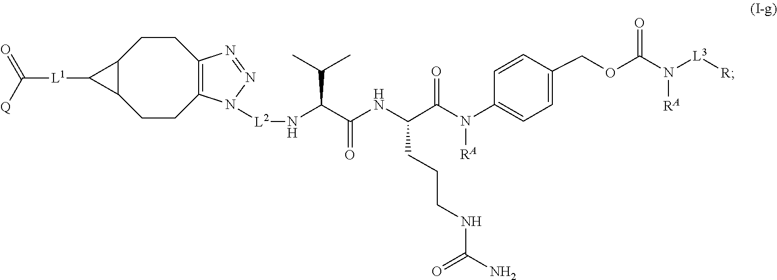

or a salt thereof, and the compound of Formula (II) is of Formula (II-g):

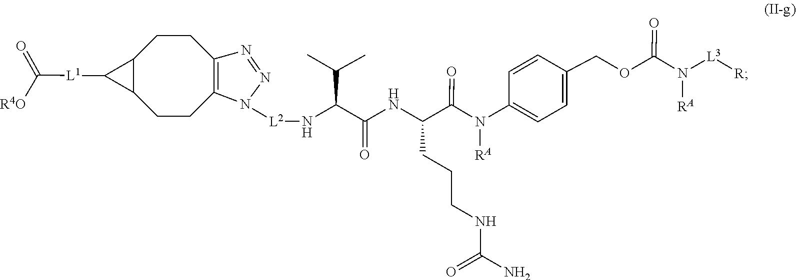

or a salt thereof.
22. The method of clause 21, further comprising reacting a compound of Formula (III-b):

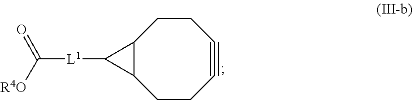

or a salt thereof, with a compound of Formula (IV-e):

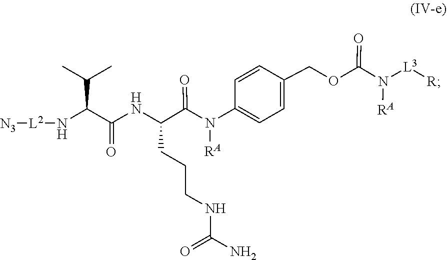

or a salt thereof, to provide a compound of Formula (II-g), or a salt thereof.
23. The method of clause 21 or 22, further comprising reacting a compound of Formula (V-d):

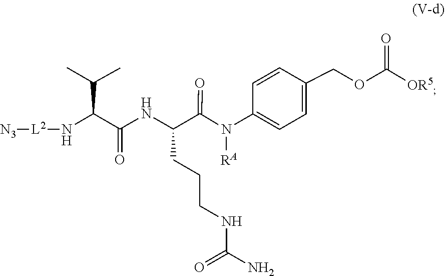

or a salt thereof, with a compound of Formula (VI-a):

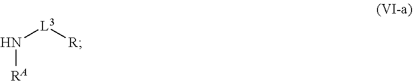

or a salt thereof, to provide a compound of Formula (IV-e), wherein:
R⁵ is substituted or unsubstituted aryl, or substituted or unsubstituted heteroaryl.
   24. The method of any of clauses 21-23, further comprising reacting a compound of Formula (VII-d):

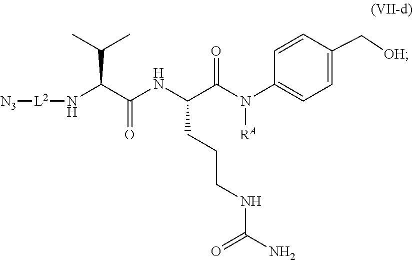

or a salt thereof, with a compound of Formula (VIII):

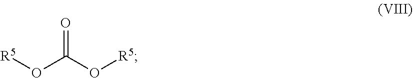

or a salt thereof, to provide a compound of Formula (V-d), wherein:
R⁵ is substituted or unsubstituted aryl, or substituted or unsubstituted heteroaryl.
   25. The method of any of clauses 21-24, further comprising reacting a compound of Formula (IX-d):

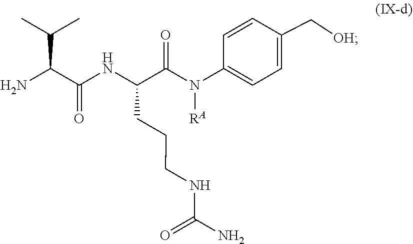

or a salt thereof, with a compound of Formula (X):

N₃-L²-LG (X);

or a salt thereof, to provide a compound of Formula (VII-d), wherein:
LG is a leaving group.
   26. The method of any of clauses 1, 6, 11, 16, or 21, wherein the compound of Formula (I) is of Formula (I-j):

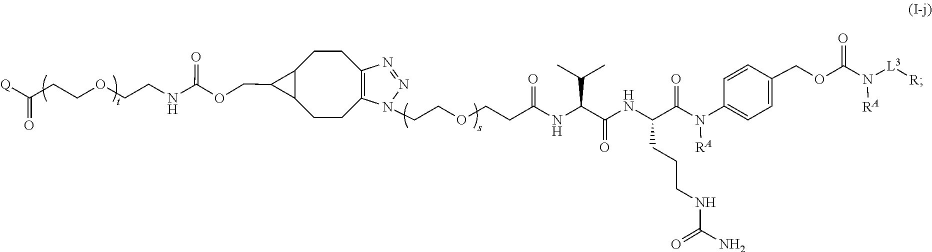

or a salt thereof, and the compound of Formula (II) is of Formula (II-j):

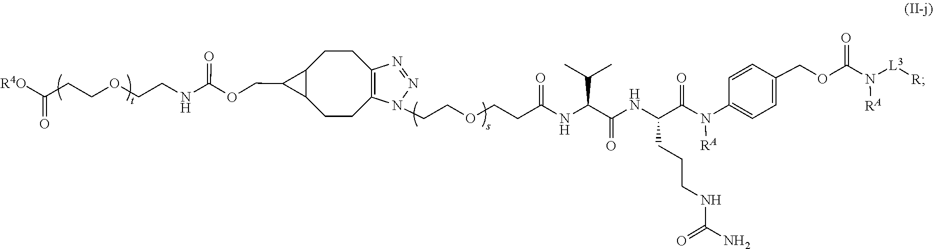

or a salt thereof, wherein:
t is 1-12; and
s is 1-12.
   27. The method of clause 26, further comprising reacting a compound of Formula (III-c):

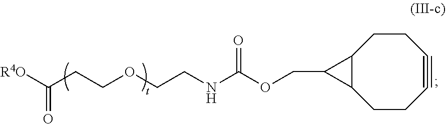

or a salt thereof, with a compound of Formula (IV-f):

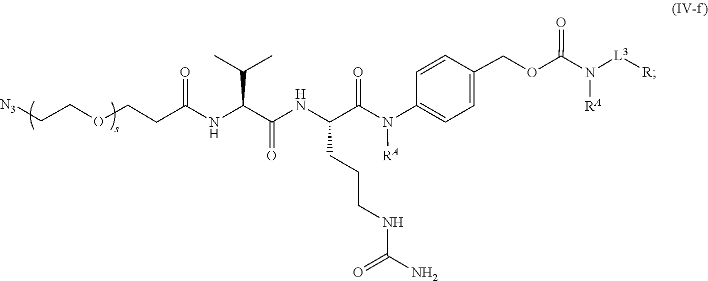

or a salt thereof, to provide a compound of Formula (II-j), or a salt thereof.
28. The method of clause 26 or 27, further comprising reacting a compound of Formula (V-e):

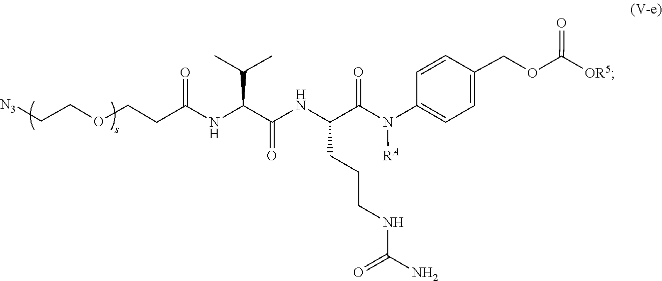

or a salt thereof, with a compound of Formula (VI-a):

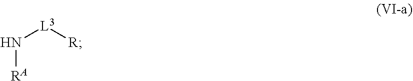

or a salt thereof, to provide a compound of Formula (IV-f), wherein:
R⁵ is substituted or unsubstituted aryl, or substituted or unsubstituted heteroaryl.
   29. The method of any of clauses 26-28, further comprising reacting a compound of Formula (VII-e):

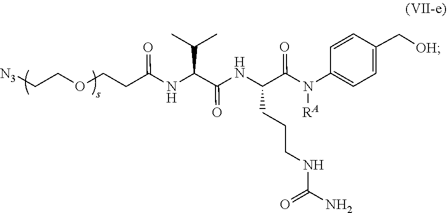

or a salt thereof, with a compound of Formula (VIII):

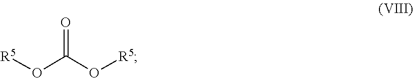

or a salt thereof, to provide a compound of Formula (V-e), wherein:
R⁵ is substituted or unsubstituted aryl, or substituted or unsubstituted heteroaryl.
   30. The method of any of clauses 26-29, further comprising reacting a compound of Formula (IX-d):

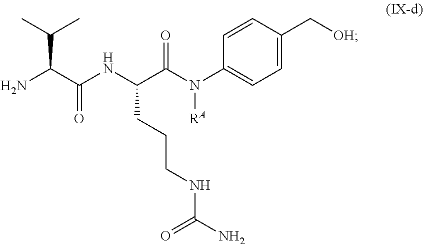

or a salt thereof, with a compound of Formula (X-a):

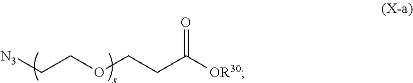

or a salt thereof, to provide a compound of Formula (VII-e), wherein:
R³⁰ is a substituted or unsubstituted heterocycle.
   31. The method of any of clauses 26-30, further comprising reacting a compound of formula:

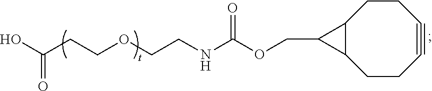

or a salt thereof, with a compound of formula R⁴-LG, wherein LG is a leaving group.
32. A method of preparing a compound of Formula (I):

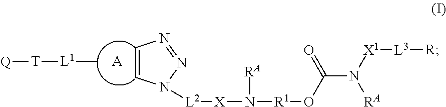

or a salt thereof, the method comprising reacting a compound of Formula (IV):

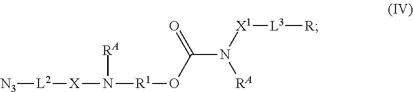

or a salt thereof, with a compound of Formula (B

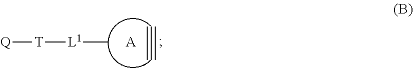

or a salt thereof, to provide a compound of Formula (I), wherein.
Q is a targeting agent;
T is

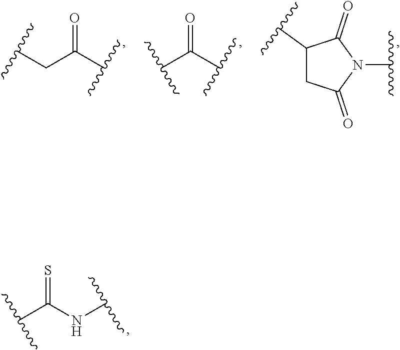

or —S—;
L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
A is substituted or unsubstituted carbocycle or substituted or unsubstituted heterocycle, or A is absent and L¹ is bonded directly to the triazole ring;
A¹ is substituted or unsubstituted carbocycle or substituted or unsubstituted heterocycle, or A¹ is absent and L¹ is bonded directly to one terminus of the alkyne;
L² is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
X is a cleavable moiety;
R¹ is substituted or unsubstituted arylene, or substituted or unsubstituted alkylene, or a combination thereof;
X¹ is a bond or a peptide;
L³ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
each R^{A} is independently hydrogen or substituted or unsubstituted alkyl; and
R is a molecular payload.
   32a. The method of clause 32, wherein the reacting of a compound of Formula (IV) with a compound of Formula (B) takes place in solvent comprising dimethyl acetamide or isopropyl alcohol.
   33. The method of clause 32, further comprising coupling a targeting agent (Q) with a compound of Formula (III):

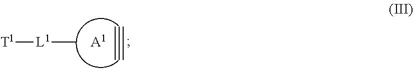

or a salt thereof, to provide a compound of Formula (B), wherein:
T¹ is

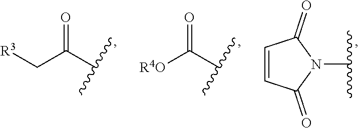

S═C═N—, or

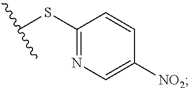 [SMILES: *Sc1ccc([N+](=O)[O-])cn1]

R³ is a leaving group; and
R⁴ is hydrogen; substituted or unsubstituted alkyl; substituted or unsubstituted heterocyclyl; substituted or unsubstituted aryl; substituted or unsubstituted heteroaryl; or an oxygen protecting group.
   34. The method of clause 32 or 33, further comprising reacting a compound of Formula (V):

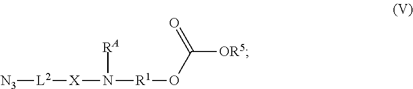

or a salt thereof, with a compound of Formula (VI):

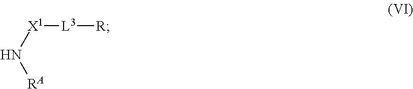

or a salt thereof, to provide a compound of Formula (IV), wherein:
R⁵ is substituted or unsubstituted aryl, or substituted or unsubstituted heteroaryl.
   35. The method of any of clauses 32-34, further comprising reacting a compound of Formula (VII):

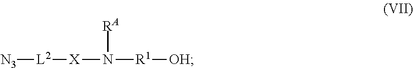

or a salt thereof, with a compound of Formula (VIII):

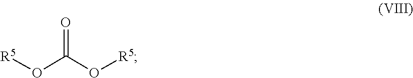

or a salt thereof, to provide a compound of Formula (V), wherein:
R⁵ is substituted or unsubstituted aryl, or substituted or unsubstituted heteroaryl.
   36. The method of any of clauses 32-35, further comprising reacting a compound of Formula (IX):

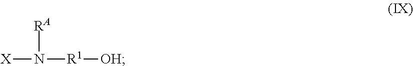

or a salt thereof, with a compound of Formula (X):

N₃-L²-LG (X);

or a salt thereof, to provide a compound of Formula (VII), wherein:
LG is a leaving group.
   37. The method of clause 32, wherein the compound of Formula (I) is of Formula (I-c):

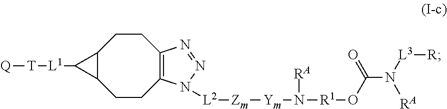

or a salt thereof, the compound of Formula (IV) is of Formula (IV-b):

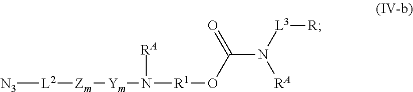

or a salt thereof, and the compound of Formula (B) is of Formula (B-a):

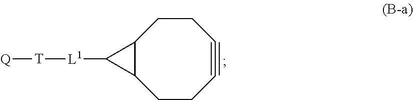

or a salt thereof, wherein:
each occurrence of Z is independently an amino acid;
each occurrence of Y is independently an amino acid; and
each m is independently 1, 2, or 3.
   38. The method of clause 37, further comprising coupling a targeting agent (Q) with a compound of Formula (III-a):

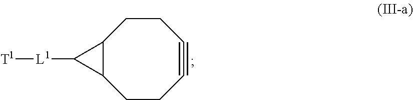

or a salt thereof, to provide a compound of Formula (B-a), wherein:
T¹ is R,

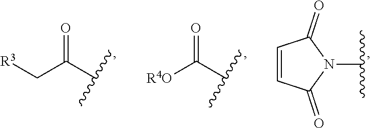

S=C═N—, or

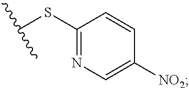 [SMILES: *Sc1ccc([N+](=O)[O-])cn1]

R³ is a leaving group; and
R⁴ is hydrogen; substituted or unsubstituted alkyl; substituted or unsubstituted heterocyclyl; substituted or unsubstituted aryl; substituted or unsubstituted heteroaryl; or an oxygen protecting group.
   39. The method of clause 37 or 38, further comprising reacting a compound of Formula (V-a):

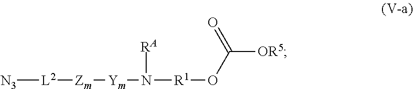

or a salt thereof, with a compound of Formula (VI-a):

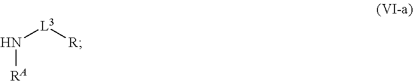

or a salt thereof, to provide a compound of Formula (IV-b), wherein:
R⁵ is substituted or unsubstituted aryl, or substituted or unsubstituted heteroaryl.
   40. The method of any of clauses 37-39, further comprising reacting a compound of Formula (VII-a):

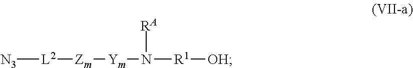

or a salt thereof, with a compound of Formula (VIII):

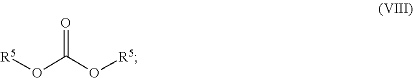

or a salt thereof, to provide a compound of Formula (V-a), wherein:
R⁵ is substituted or unsubstituted aryl, or substituted or unsubstituted heteroaryl.
   41. The method of any of clauses 37-40, further comprising reacting a compound of Formula (IX-a):

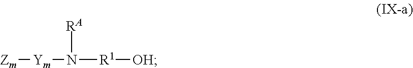

or a salt thereof, with a compound of Formula (X):

N₃-L²-LG (X);

or a salt thereof, to provide a compound of Formula (VII-a), wherein:
LG is a leaving group and is displaced by the amino group of the terminal amino acid Z.
   42. The method of clause 32 or 37, wherein the compound of Formula (I) is of Formula (I-e):

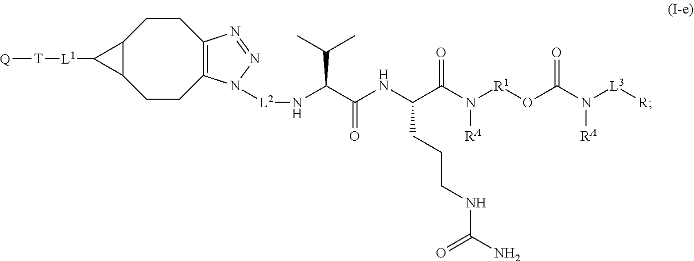

or a salt thereof, the compound of Formula (IV) is of Formula (IV-d):

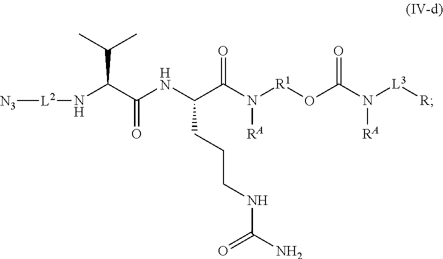

or a salt thereof, and the compound of Formula (B) is of Formula (B-a):

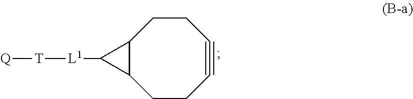

or a salt thereof.
43. The method of clause 42, further comprising coupling a targeting agent (Q) with a compound of Formula (III-a):

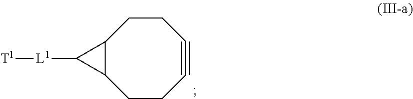

or a salt thereof, to provide a compound of Formula (B-a), wherein:
T¹ is

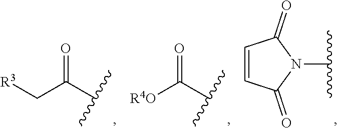

S═C═N—, or

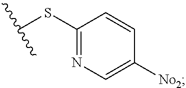

R³ is a leaving group; and
R⁴ is hydrogen; substituted or unsubstituted alkyl; substituted or unsubstituted heterocyclyl; substituted or unsubstituted aryl; substituted or unsubstituted heteroaryl; or an oxygen protecting group.
   44. The method of clause 42 or 43, further comprising reacting a compound of Formula (V-C):

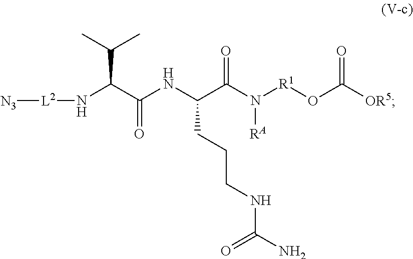

or a salt thereof, with a compound of Formula (VI-a):

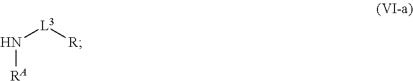

or a salt thereof, to provide a compound of Formula (IV-d), wherein:
R⁵ is substituted or unsubstituted aryl, or substituted or unsubstituted heteroaryl.
   45. The method of any of clauses 42-44, further comprising reacting a compound of Formula (VII-c):

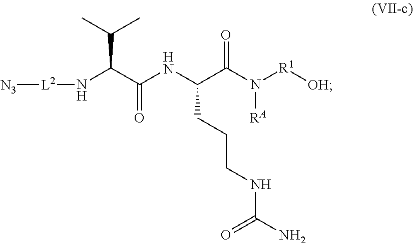

or a salt thereof, with a compound of Formula (VIII):

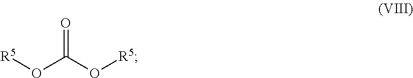

or a salt thereof, to provide a compound of Formula (V-c), wherein:
R⁵ is substituted or unsubstituted aryl, or substituted or unsubstituted heteroaryl.
   46. The method of any of clauses 42-45, further comprising reacting a compound of Formula (IX-c):

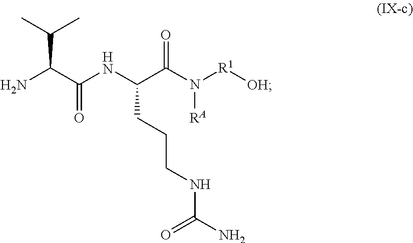

or a salt thereof, with a compound of Formula (X):

N₃-L²-LG (X);

or a salt thereof, to provide a compound of Formula (VII-c), wherein:
LG is a leaving group.
   47. The method of clause 32, 37, or 42, wherein the compound of Formula (I) is of Formula (I-f):

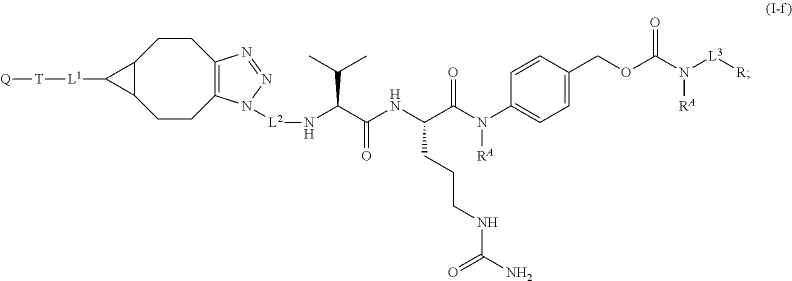

or a salt thereof, the compound of Formula (IV) is of Formula (IV-e):

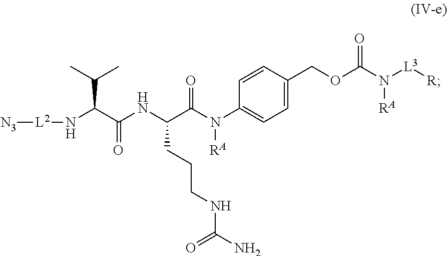

or a salt thereof, and the compound of Formula (B) is of Formula (B-a):

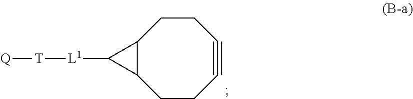

or a salt thereof.
48. The method of clause 47, further comprising coupling a targeting agent (Q) with a compound of Formula (III-a):

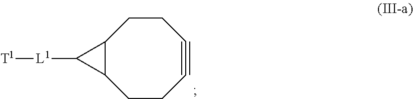

or a salt thereof, to provide a compound of Formula (B-a), wherein:
T¹ is

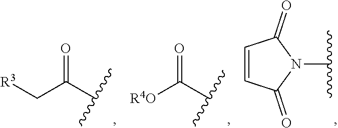

S═C═N—, or

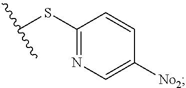

R³ is a leaving group; and
R⁴ is hydrogen; substituted or unsubstituted alkyl; substituted or unsubstituted heterocyclyl; substituted or unsubstituted aryl; substituted or unsubstituted heteroaryl; or an oxygen protecting group.
   49. The method of clause 47 or 48, further comprising reacting a compound of Formula (V-d):

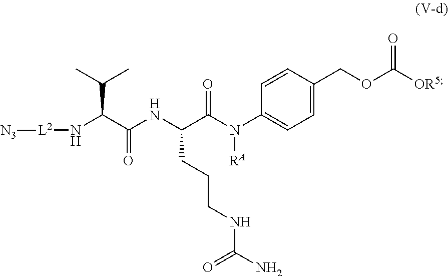

or a salt thereof, with a compound of Formula (VI-a):

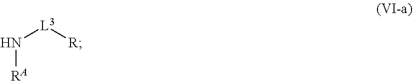

or a salt thereof, to provide a compound of Formula (IV-e), wherein:
R⁵ is substituted or unsubstituted aryl, or substituted or unsubstituted heteroaryl.
   50. The method of any of clauses 47-49, further comprising reacting a compound of Formula

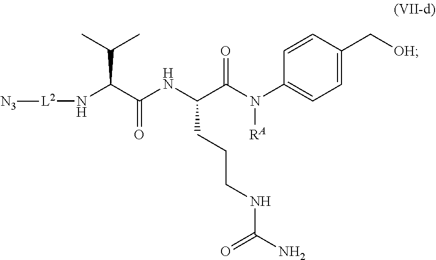

or a salt thereof, with a compound of Formula (VIII):

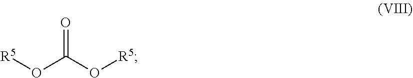

or a salt thereof, to provide a compound of Formula (V-d), wherein:
R⁵ is substituted or unsubstituted aryl, or substituted or unsubstituted heteroaryl.
   51. The method of any of clauses 47-50, further comprising reacting a compound of Formula (IX-d):

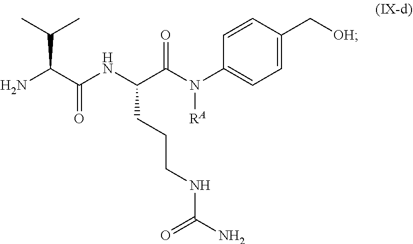

or a salt thereof, with a compound of Formula (X):

N₃-L²-LG (X);

or a salt thereof, to provide a compound of Formula (VII-d), wherein:
LG is a leaving group.
   52. The method of clause 32, 37, 42, or 47, wherein the compound of Formula (I) is of Formula (I-g):

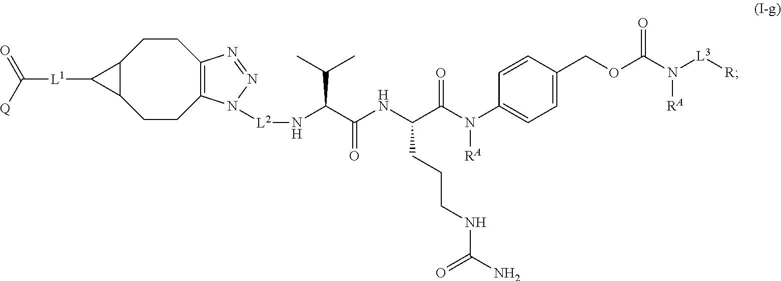

or a salt thereof, the compound of Formula (IV) is of Formula (IV-e):

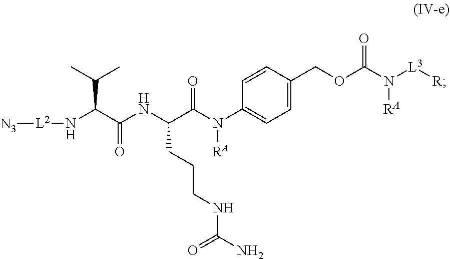

or a salt thereof, and the compound of Formula (B) is of Formula (B-b):

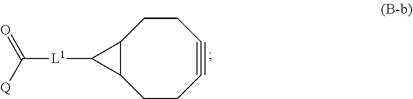

or a salt thereof.
53. The method of clause 52, further comprising coupling a targeting agent (Q) with a compound of Formula (III-b):

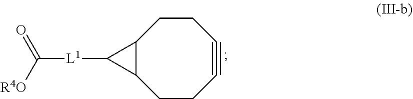

or a salt thereof, to provide a compound of Formula (B-b), or a salt thereof.
54. The method of clause 52 or 53, further comprising reacting a compound of Formula (V-d):

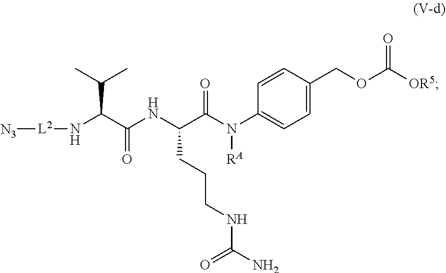

or a salt thereof, with a compound of Formula (VI-a):

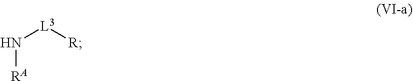

or a salt thereof, to provide a compound of Formula (IV-e), wherein:
R⁵ is substituted or unsubstituted aryl, or substituted or unsubstituted heteroaryl.
   55. The method of any of clauses 52-54, further comprising reacting a compound of Formula (VII-d):

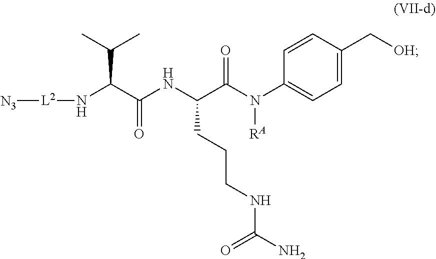

or a salt thereof, with a compound of Formula (VIII):

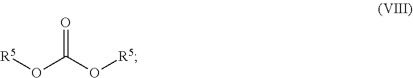

or a salt thereof, to provide a compound of Formula (V-d), wherein:
R⁵ is substituted or unsubstituted aryl, or substituted or unsubstituted heteroaryl.
   56. The method of any of clauses 52-55, further comprising reacting a compound of Formula (IX-d):

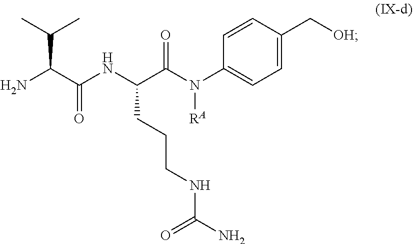

or a salt thereof, with a compound of Formula (X):

N₃-L²-LG (X);

or a salt thereof, to provide a compound of Formula (VII-d), wherein:
LG is a leaving group.
   57. The method of clause 32, 37, 42, 47, or 52, wherein the compound of Formula (I) is of Formula (I-j):

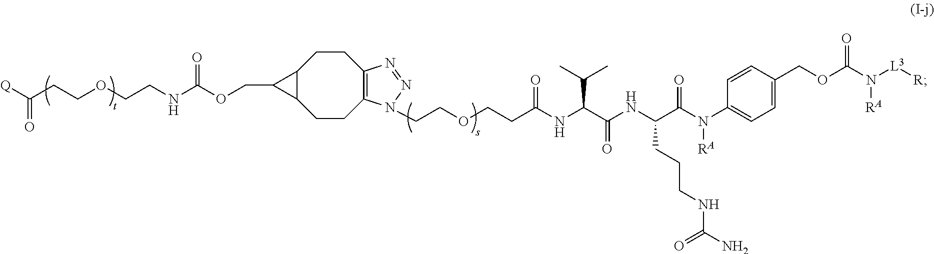

or a salt thereof, the compound of Formula (IV) is of Formula (IV-f):

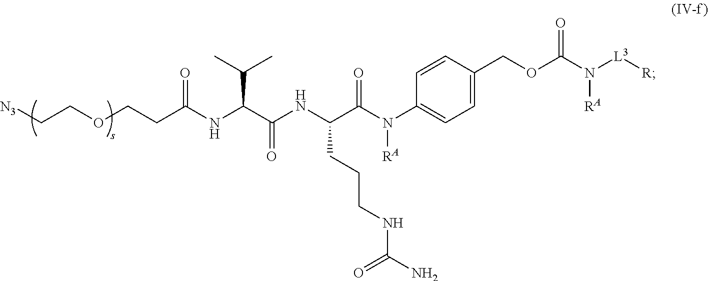

or a salt thereof, and the compound of Formula (B) is of Formula (B-c):

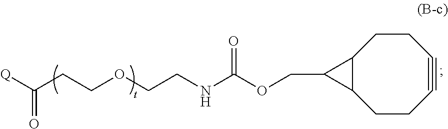

or a salt thereof, wherein:
t is 1-12.
   58. The method of clause 57, further comprising coupling a targeting agent (Q) with a compound of Formula (III-c):

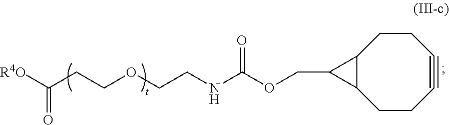

or a salt thereof, to provide a compound of Formula (B-c), or a salt thereof.
59. The method of clause 57 or 58, further comprising reacting a compound of Formula (V-e):

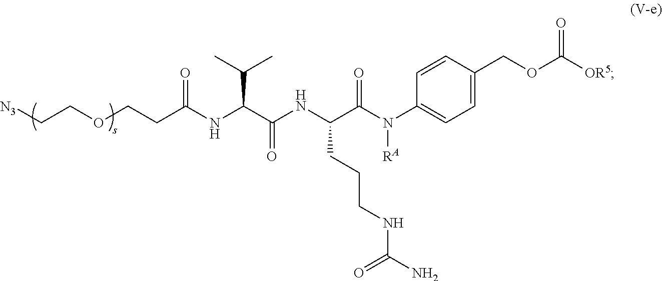

or a salt thereof, with a compound of Formula (VI-a):

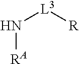

or a salt thereof, to provide a compound of Formula (IV-f), wherein:
R⁵ is substituted or unsubstituted aryl, or substituted or unsubstituted heteroaryl.
   60. The method of any of clauses 57-59, further comprising reacting a compound of Formula (VII-e):

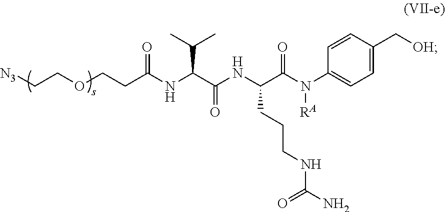

or a salt thereof, with a compound of Formula (VIII):

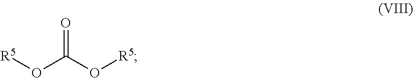

or a salt thereof, to provide a compound of Formula (V-e), wherein:
R⁵ is substituted or unsubstituted aryl, or substituted or unsubstituted heteroaryl.
   61. The method of any of clauses 57-60, further comprising reacting a compound of Formula (IX-d):

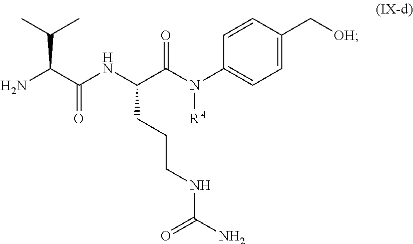

or a salt thereof, with a compound of Formula (X-a):

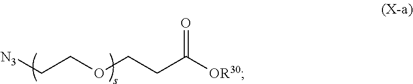

or a salt thereof, to provide a compound of Formula (VII-e), wherein:
R³⁰ is a substituted or unsubstituted heterocycle.
   62. The method of any of clauses 57-61, further comprising reacting a compound of formula:

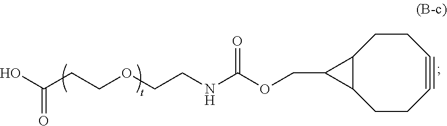

or a salt thereof, with a compound of formula R⁴-LG to form the compound of Formula (III-c), wherein LG is a leaving group.
63. A method of preparing a compound of Formula (I):

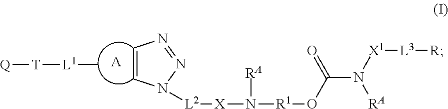

or a salt thereof, the method comprising reacting a targeting agent (Q); a compound of Formula (IV):

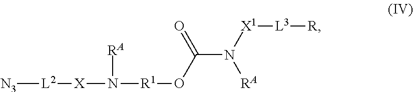

or a salt thereof; and a compound of Formula (III):

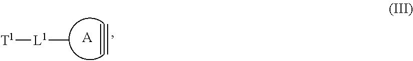

or a salt thereof; to provide a compound of Formula (I), or a salt thereof, wherein:
T is

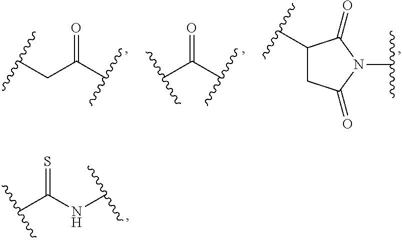

or —S—;
T¹ is

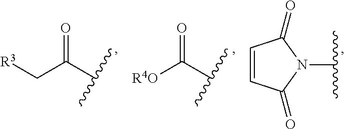

S═C═N—, or

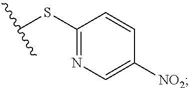 [SMILES: *Sc1ccc([N+](=O)[O-])cn1]

R³ is a leaving group (e.g., halogen, tosylate, mesylate, or triflate);
R⁴ is hydrogen; substituted or unsubstituted alkyl; substituted or unsubstituted heterocyclyl; substituted or unsubstituted aryl; substituted or unsubstituted heteroaryl; or an oxygen protecting group;
L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
A is substituted or unsubstituted carbocycle or substituted or unsubstituted heterocycle, or A is absent and L¹ is bonded directly to the triazole ring;
A¹ is substituted or unsubstituted carbocycle or substituted or unsubstituted heterocycle, or A¹ is absent and L¹ is bonded directly to one terminus of the alkyne;
L² is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
X is a cleavable moiety;
R¹ is substituted or unsubstituted arylene, or substituted or unsubstituted alkylene, or a combination thereof;
X¹ is a bond or a peptide;
L³ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
each R^{A} is independently hydrogen or substituted or unsubstituted alkyl; and
R is a molecular payload.
   64. The method of clause 63, further comprising reacting a compound of Formula (V):

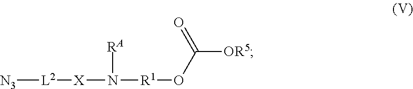

or a salt thereof, with a compound of Formula (VI):

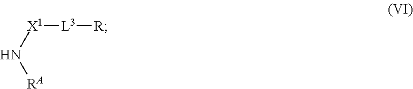

or a salt thereof, to provide a compound of Formula (IV), wherein:
R⁵ is substituted or unsubstituted aryl, or substituted or unsubstituted heteroaryl.
   65. The method of clause 63 or 64, further comprising reacting a compound of Formula (VII):

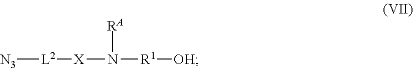

or a salt thereof, with a compound of Formula (VIII):

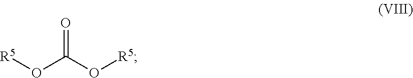

or a salt thereof, to provide a compound of Formula (V), wherein:
R⁵ is substituted or unsubstituted aryl, or substituted or unsubstituted heteroaryl.
   66. The method of any of clauses 63-65, further comprising reacting a compound of Formula (IX):

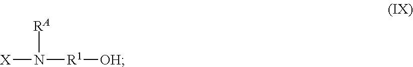

or a salt thereof, with a compound of Formula (X):

N₃-L²-LG (X);

or a salt thereof, to provide a compound of Formula (VII), wherein:
LG is a leaving group.
   67. The method of clause 63, wherein the compound of Formula (I) is of Formula (I-c):

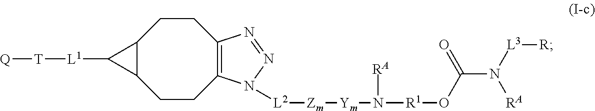

or a salt thereof, the compound of Formula (IV) is of Formula (IV-b):

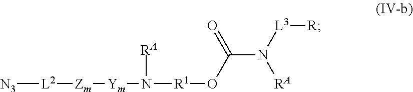

or a salt thereof, and the compound of Formula (III) is of Formula (III-a):

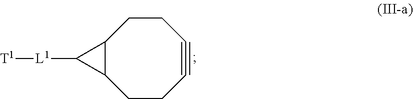

or a salt thereof, wherein:
each occurrence of Z is independently an amino acid;
each occurrence of Y is independently an amino acid; and
each m is independently 1, 2, or 3.
   68. The method of clause 67, further comprising reacting a compound of Formula (V-a):

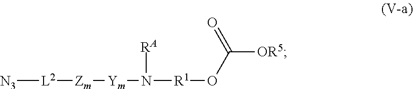

or a salt thereof, with a compound of Formula (VI-a):

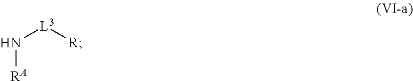

or a salt thereof, to provide a compound of Formula (IV-b), wherein:
R⁵ is substituted or unsubstituted aryl, or substituted or unsubstituted heteroaryl.
   69. The method of clause 67 or 68, further comprising reacting a compound of Formula (VII-a):

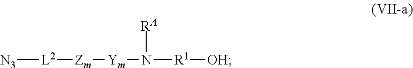

or a salt thereof, with a compound of Formula (VIII):

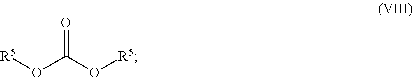

or a salt thereof, to provide a compound of Formula (V-a), wherein:
R⁵ is substituted or unsubstituted aryl, or substituted or unsubstituted heteroaryl.
   70. The method of any of clauses 67-69, further comprising reacting a compound of Formula (IX-a):

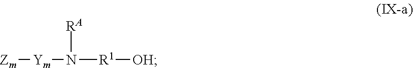

or a salt thereof, with a compound of Formula (X):

N₃-L²-LG (X);

or a salt thereof, to provide a compound of Formula (VII-a), wherein:
LG is a leaving group and is displaced by the amino group of the terminal amino acid Z.
   71. The method of clause 63 or 67, wherein the compound of Formula (I) is of Formula (I-e):

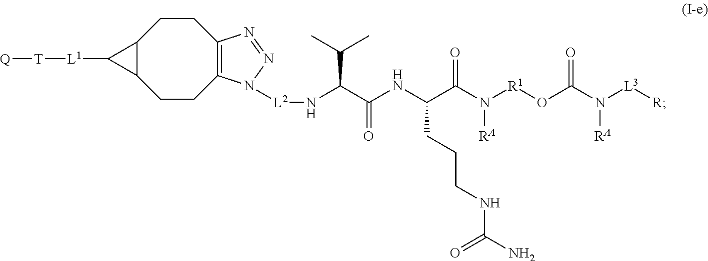

or a salt thereof, the compound of Formula (IV) is of Formula (IV-d):

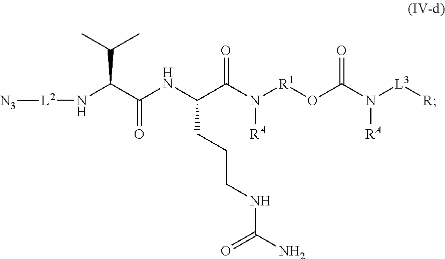

or a salt thereof, and the compound of Formula (III) is of Formula (III-a):

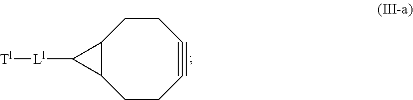

or a salt thereof.
72. The method of clause 71, further comprising reacting a compound of Formula (V-c):

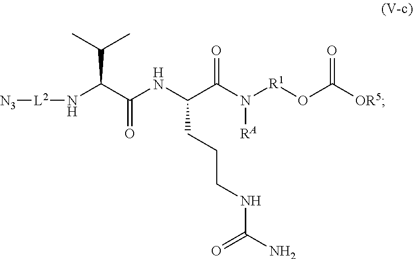

or a salt thereof, with a compound of Formula (VI-a):

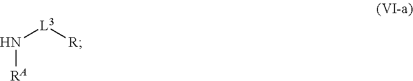

or a salt thereof, to provide a compound of Formula (IV-d), wherein:
R⁵ is substituted or unsubstituted aryl, or substituted or unsubstituted heteroaryl.
   73. The method of clause 71 or 72, further comprising reacting a compound of Formula (VII-c):

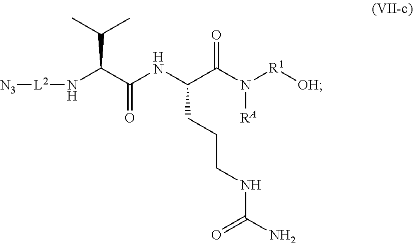

or a salt thereof, with a compound of Formula (VIII):

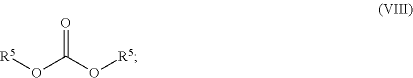

or a salt thereof, to provide a compound of Formula (V-c), wherein:
R⁵ is substituted or unsubstituted aryl, or substituted or unsubstituted heteroaryl.
   74. The method of any of clauses 71-73, further comprising reacting a compound of Formula (IX-c):

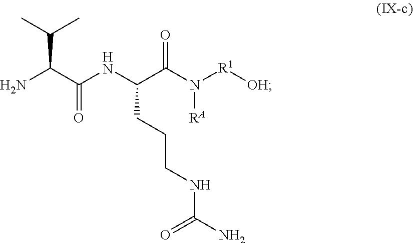

or a salt thereof, with a compound of Formula (X):

N₃-L²-LG (X);

or a salt thereof, to provide a compound of Formula (VII-c), wherein:
LG is a leaving group.
   75. The method of clause 63, 67, or 71, wherein the compound of Formula (I) is of Formula (I-f):

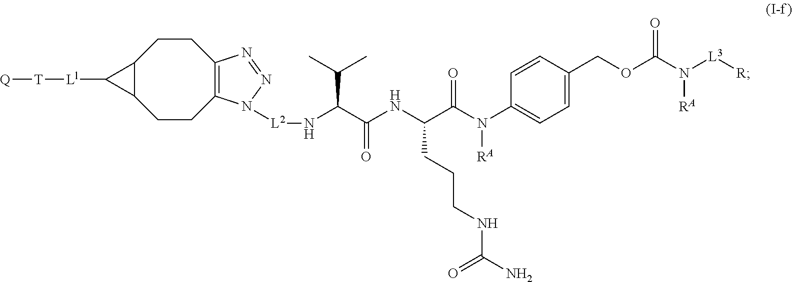

or a salt thereof, the compound of Formula (IV) is of Formula (IV-e):

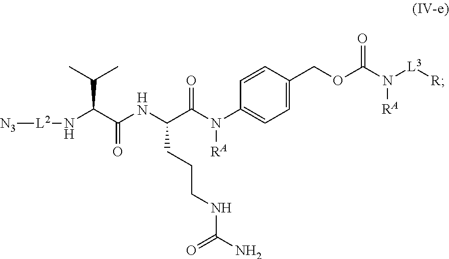

or a salt thereof, and the compound of Formula (III) is of Formula (III-a):

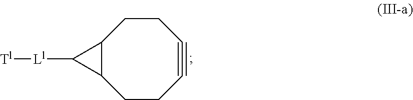

or a salt thereof.
76. The method of clause 75, further comprising reacting a compound of Formula (V-d):

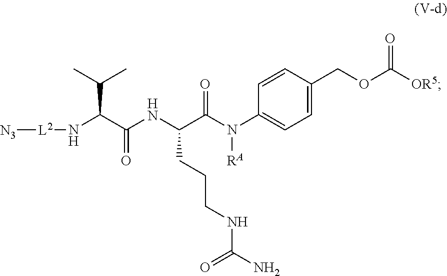

or a salt thereof, with a compound of Formula (VI-a):

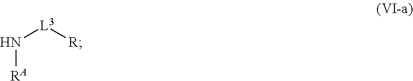

or a salt thereof, to provide a compound of Formula (IV-e), wherein:
R⁵ is substituted or unsubstituted aryl, or substituted or unsubstituted heteroaryl.
   77. The method of clause 75 or 76, further comprising reacting a compound of Formula (VII-d):

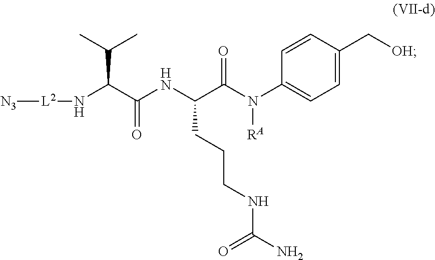

or a salt thereof, with a compound of Formula (VIII):

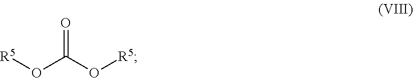

or a salt thereof, to provide a compound of Formula (V-d), wherein:
R⁵ is substituted or unsubstituted aryl, or substituted or unsubstituted heteroaryl.
   78. The method of any of clauses 75-77, further comprising reacting a compound of Formula (IX-d):

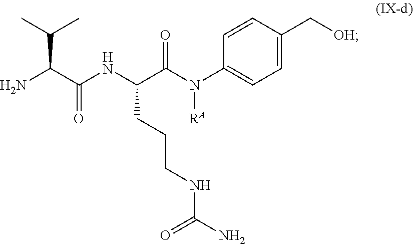

or a salt thereof, with a compound of Formula (X):

N₃-L²-LG (X);

or a salt thereof, to provide a compound of Formula (VII-d), wherein:
LG is a leaving group.
   79. The method of clause 63, 67, 71, or 75, wherein the compound of Formula (I) is of Formula (I-g):

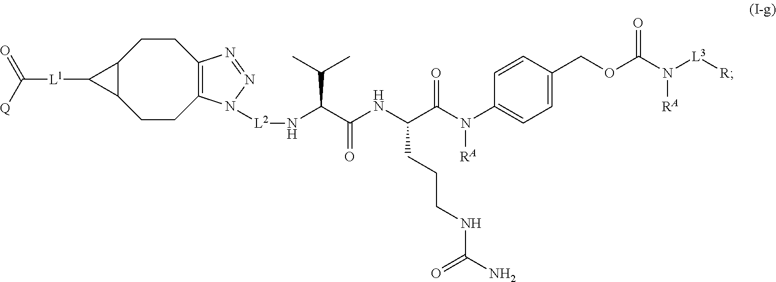

or a salt thereof, the compound of Formula (IV) is of Formula (IV-d):

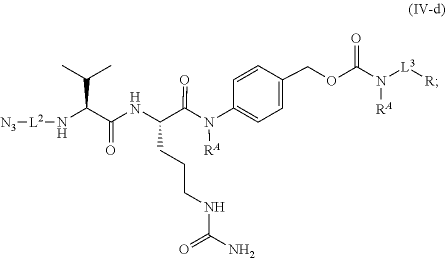

or a salt thereof, and the compound of Formula (III) is of Formula (III-b):

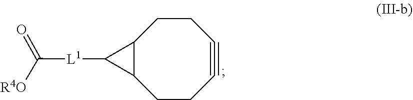

or a salt thereof.
80. The method of clause 79, further comprising reacting a compound of Formula (V-d):

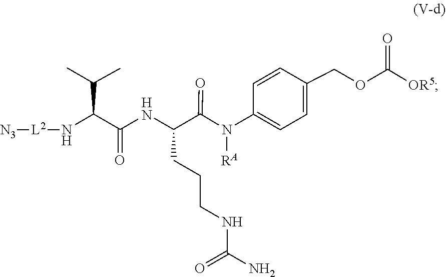

or a salt thereof, with a compound of Formula (VI-a):

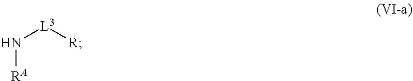

or a salt thereof, to provide a compound of Formula (IV-d), wherein:
R⁵ is substituted or unsubstituted aryl, or substituted or unsubstituted heteroaryl.
   81. The method of clause 79 or 80, further comprising reacting a compound of Formula (VII-d):

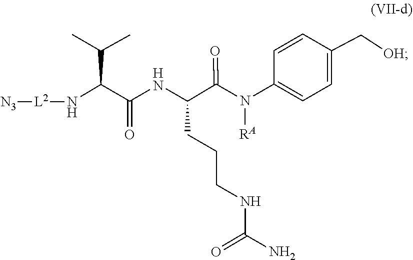

or a salt thereof, with a compound of Formula (VIII):

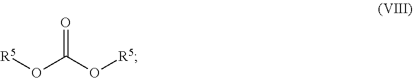

or a salt thereof, to provide a compound of Formula (V-d), wherein:
R⁵ is substituted or unsubstituted aryl, or substituted or unsubstituted heteroaryl.
   82. The method of any of clauses 79-81, further comprising reacting a compound of Formula (IX-d):

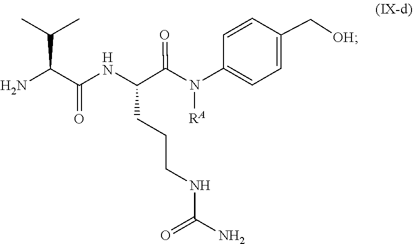

or a salt thereof, with a compound of Formula (X):

N₃-L²-LG (X);

or a salt thereof, to provide a compound of Formula (VII-d), wherein:
LG is a leaving group.
   83. The method of clause 63, 67, 71, 75, or 79, wherein the compound of Formula (I) is of Formula (I-k):

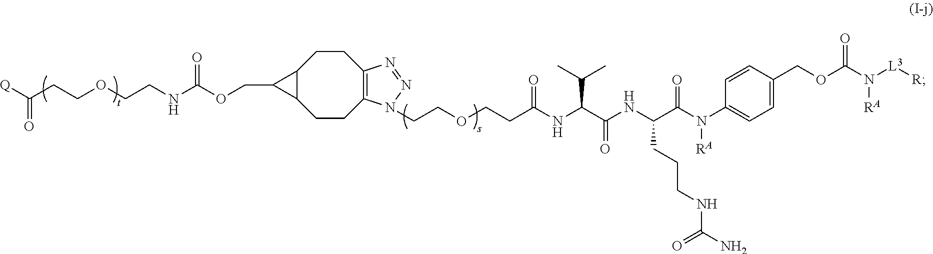

or a salt thereof, the compound of Formula (IV) is of Formula (IV-f):

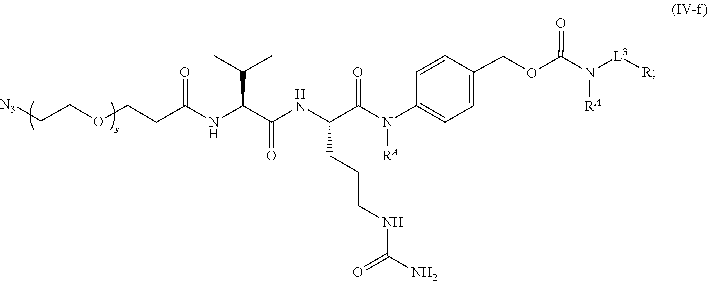

or a salt thereof, and the compound of Formula (III) is of Formula (III-c):

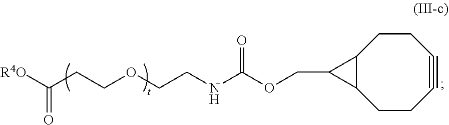

or a salt thereof, wherein:
s is 1-12; and
t is 1-12.
   84. The method of clause 83, further comprising reacting a compound of Formula (V-e):

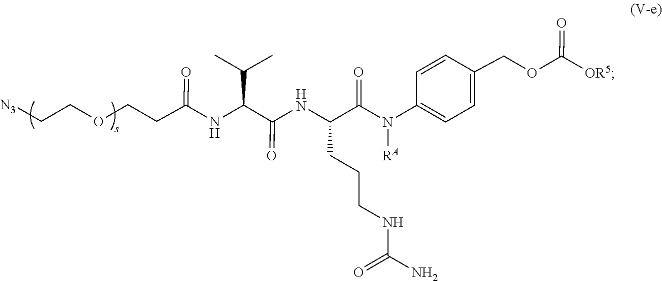

or a salt thereof, with a compound of Formula (VI-a):

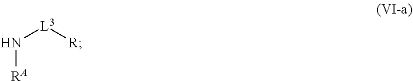

or a salt thereof, to provide a compound of Formula (IV-f)), wherein:
R⁵ is substituted or unsubstituted aryl, or substituted or unsubstituted heteroaryl.
   85. The method of clause 83 or 84, further comprising reacting a compound of Formula (VII-e):

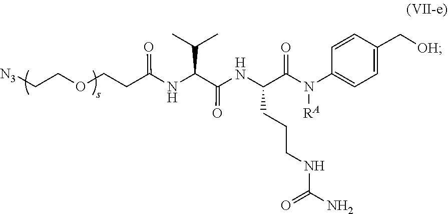

or a salt thereof, with a compound of Formula (VIII):

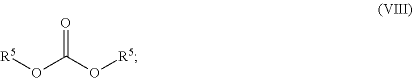

or a salt thereof, to provide a compound of Formula (V-e), wherein:
R⁵ is substituted or unsubstituted aryl, or substituted or unsubstituted heteroaryl.
   86. The method of any of clauses 83-85, further comprising reacting a compound of Formula (IX-d):

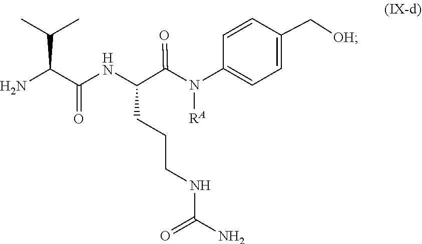

or a salt thereof, with a compound of Formula (X-a):

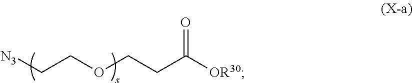

or a salt thereof, to provide a compound of Formula (VII-e), wherein:
R³⁰ is a substituted or unsubstituted heterocycle.
   87. The method of any of clauses 83-86, further comprising reacting a compound of formula:

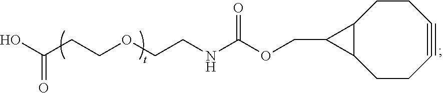

or a salt thereof, with a compound of formula R⁴-LG, to form a compound of Formula (III-c), or a salt thereof, wherein LG is a leaving group.
88. The method of any of clauses 1-87, wherein Q is an antibody.
89. The method of any of clauses 1-87, wherein Q is antibody, which is a full-length IgG, a Fab fragment, a Fab′ fragment, a F(ab′)2 fragment, a scFv, or a Fv fragment.
90. The method of any of clauses 1-89, wherein Q is an anti-transferrin receptor antibody.
91. The method of any of clauses 1-90, wherein R is an oligonucleotide.
92. The method of any of clauses 1-91, wherein R is charge-neutral oligonucleotide.
93. The method of any of clauses 1-92, wherein R is a single-stranded oligonucleotide.
94. The method of any of clauses 1-93, wherein R is an antisense oligonucleotide.
95. The method of any of clauses 1-94, wherein R is a phosphorodiamidate morpholino oligomer (PMO).
96. The method of any of clauses 1-95, wherein R is an oligonucleotide of 10-50 nucleotides in length.
97. The method of any of clauses 1-96, wherein R is an oligonucleotide of 20-30 nucleotides in length.
98. The method of any of clauses 1-97, wherein R is an oligonucleotide bound to the compound by the 5′ of the oligonucleotide.
99. The method of any clauses 1-98, wherein R is an oligonucleotide bound to the compound by the 3′ of the oligonucleotide.
100. A compound of Formula (I):

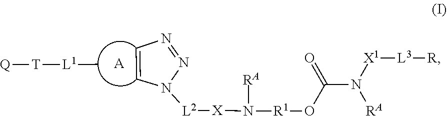

or a pharmaceutically acceptable salt, tautomer, stereoisomer, or isotopically enriched derivative thereof, wherein:
Q is a targeting agent;
T is

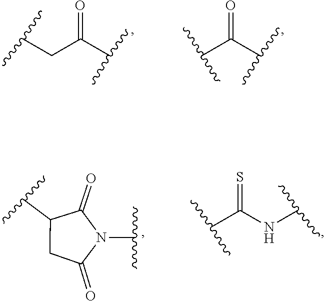

or —S—;
L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
A is substituted or unsubstituted carbocycle or substituted or unsubstituted heterocycle, or A is absent and L¹ is bonded directly to the triazole ring;
L² is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
X is a cleavable moiety;
R¹ is substituted or unsubstituted arylene, or substituted or unsubstituted alkylene, or a combination thereof;
X¹ is a bond or a peptide;
L³ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
each R^{A} is independently hydrogen or substituted or unsubstituted alkyl; and
R is a molecular payload.
   101. The compound of clause 100, or a salt thereof, wherein:
T is:

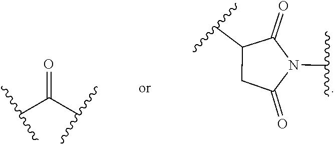

102. The compound of clause 100 or 101, or a salt thereof, wherein:
T is:

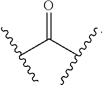 [SMILES: *C(*)=O]

103. The compound of any of clauses 100-102, or a salt thereof, wherein:
L¹ is —C(═O)—, —O—, —NR^{A}—, substituted or unsubstituted alkylene, substituted or unsubstituted heteroalkylene, substituted or unsubstituted carbocyclylene, substituted or unsubstituted heterocyclylene, substituted or unsubstituted heteroarylene, or substituted or unsubstituted arylene, or a combination thereof.
   104. The compound of any of clauses 100-103, or a salt thereof, wherein:
L¹ is —C(═O)—, —O—, —NR^{A}—, substituted or unsubstituted alkylene, or substituted or unsubstituted heteroalkylene, or a combination thereof.
   105. The compound of any of clauses 100-104, or a salt thereof, wherein:
L¹ is

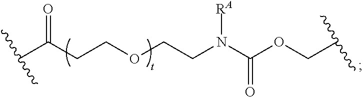

wherein t is 1-12.
106. The compound of any of clauses 100-105, or a salt thereof, wherein:
A is substituted or unsubstituted C₅10 carbocycle.
   107. The compound of any of clauses 100-106, or a salt thereof, wherein:
A is a substituted or unsubstituted bicyclic fused C₉ carbocycle.
   108. The compound of any of clauses 100-107, or a salt thereof, wherein:
A is

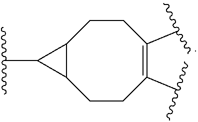 [SMILES: */C1=C(\*)CCC2C(*)C2CC1]

109. The compound of any of clauses 100-108, or a salt thereof, wherein:
L² is —C(═O)—, —O—, —NR^{A}—, substituted or unsubstituted alkylene, substituted or unsubstituted heteroalkylene, substituted or unsubstituted carbocyclylene, substituted or unsubstituted heterocyclylene, substituted or unsubstituted heteroarylene, or substituted or unsubstituted arylene, or a combination thereof.
   110. The compound of any of clauses 100-109, or a salt thereof, wherein:
L² is —C(═O)—, substituted or unsubstituted alkylene, or substituted or unsubstituted heteroalkylene, or a combination thereof.
   111. The compound of any of clauses 100-110, or a salt thereof, wherein:
L² is

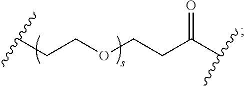

wherein s is 1-12.
   112. The compound of any of clauses 100-111, or a salt thereof, wherein:
X is —ZₘYₘ—;
each occurrence of Z is independently an amino acid;
each occurrence of Y is independently an amino acid; and
each m is independently 1, 2, or 3.
   113. The compound of clause 112, or a salt thereof, wherein:
Z is valine, and Y is citrulline.
   114. The compound of clause 112 or 113, or a salt thereof, wherein:
each m is 1.
   115. The compound of any of clauses 100-114, or a salt thereof, wherein:
X is

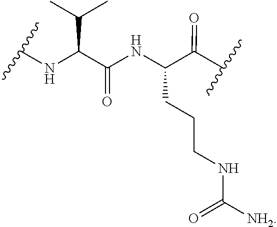 [SMILES: *N[C@H](C(=O)N[C@@H](CCCNC(N)=O)C(*)=O)C(C)C]

116. The compound of any of clauses 100-115, or a salt thereof, wherein:
R¹ is substituted or unsubstituted phenylene, or substituted or unsubstituted C₁₋₄ alkylene, or a combination thereof.
   117. The compound of any of clauses 100-116, or a salt thereof, wherein:
R¹ is a combination of substituted or unsubstituted phenylene and substituted or unsubstituted C₁₋₆ alkylene.
   118. The compound of any of clauses 100-117, or a salt thereof, wherein:
R¹ is

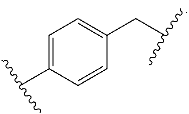 [SMILES: *Cc1ccc(*)cc1]

119. The compound of any of clauses 100-118, or a salt thereof, wherein:
L³ is substituted or unsubstituted alkylene, substituted or unsubstituted heteroalkylene, substituted or unsubstituted heterocyclylene, substituted or unsubstituted heteroarylene, —O—, —N(R^{A})—, —C(═O)—, or —C(═O)N(R^{A})₂, or a combination thereof.
   120. The compound of any of clauses 100-119, or a salt thereof, wherein:
L³ is

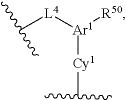

wherein:
L⁴ is —O—, substituted or unsubstituted heteroalkylene, or substituted or unsubstituted alkylene, or a combination thereof;
Cy¹ is substituted or unsubstituted heterocyclylene;
Ar¹ is substituted or unsubstituted heteroarylene; and
R⁵⁰ is substituted or unsubstituted heteroalkylene, substituted or unsubstituted alkylene, —N(R^{A})—, or —C(═O)N(R^{A})₂, or a combination thereof.
   121. The compound of any of clauses 100-120, or a salt thereof, wherein:
L³ is

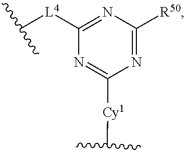

wherein:
L⁴ is —O—, substituted or unsubstituted heteroalkylene, or substituted or unsubstituted alkylene, or a combination thereof;
Cy¹ is substituted or unsubstituted heterocyclylene; and
R⁵⁰ is substituted or unsubstituted heteroalkylene, substituted or unsubstituted alkylene, —N(R^{A})—, or —C(═O)N(R^{A})₂, or a combination thereof.
   122. The compound of any of clauses 100-121, or a salt thereof, wherein:
L³ is

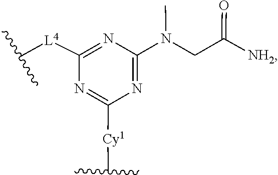

wherein:
L⁴ is —O—, substituted or unsubstituted heteroalkylene, or substituted or unsubstituted alkylene, or a combination thereof; and
Cy¹ is substituted or unsubstituted heterocyclylene.
   123. The compound of any of clauses 100-122, or a salt thereof, wherein:

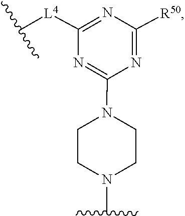

L³ is
   wherein:
L⁴ is —O—, substituted or unsubstituted heteroalkylene, or substituted or unsubstituted alkylene, or a combination thereof; and
R⁵⁰ is substituted or unsubstituted heteroalkylene, substituted or unsubstituted alkylene, —N(R^{A})—, or —C(═O)N(R^{A})₂, or a combination thereof.
   124. The compound of any of clauses 100-123, or a salt thereof, wherein:
L³ is

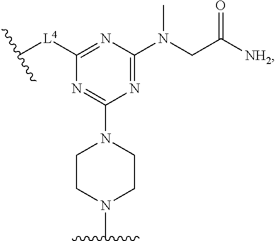

wherein:
L⁴ is —O—, substituted or unsubstituted heteroalkylene, or substituted or unsubstituted alkylene, or a combination thereof.
   125. The compound of any of clauses 100-124, or a salt thereof, wherein:
L³ is

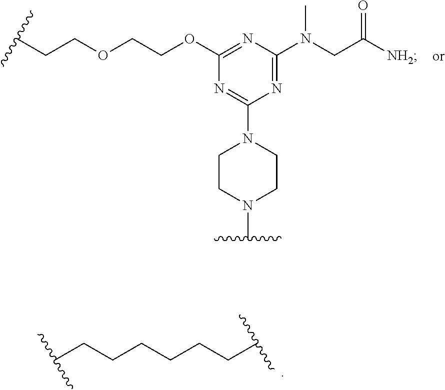 [SMILES: *CCOCCOc1nc(N(C)CC(N)=O)nc(N2CCN(*)CC2)n1 ; *CCCCCC*]

126. The compound of any of clauses 100-125, or a salt thereof, wherein:
L³ is

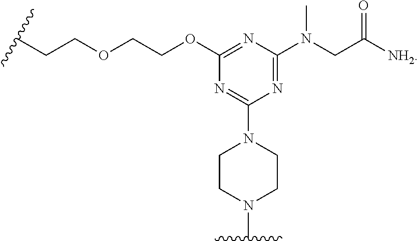 [SMILES: *CCOCCOc1nc(N(C)CC(N)=O)nc(N2CCN(*)CC2)n1]

127. The compound of any of clauses 100-126, or a salt thereof, wherein:
X¹ is a bond.
   128. The compound of any of clauses 100-127, wherein Q is an antibody.
   129. The compound of any of clauses 100-128, wherein Q is antibody, which is a full-length IgG, a Fab fragment, a Fab′ fragment, a F(ab′)2 fragment, a scFv, or a Fv fragment.
   130. The compound of any of clauses 100-129, wherein Q is an anti-transferrin receptor antibody.
   131. The compound of any of clauses 100-130, wherein R is an oligonucleotide.
   132. The compound of any of clauses 100-131, wherein R is charge-neutral oligonucleotide.
   133. The compound of any of clauses 100-132, wherein R is a single-stranded oligonucleotide.
   134. The compound of any of clauses 100-133, wherein R is an antisense oligonucleotide.
   135. The compound of any of clauses 100-134, wherein R is a phosphorodiamidate morpholino oligomer (PMO).
   136. The compound of any of clauses 100-135, wherein R is an oligonucleotide of 10-50 nucleotides in length.
   137. The compound of any of clauses 100-136, wherein R is an oligonucleotide of 20-30 nucleotides in length.
   138. The compound of any of clauses 100-137, wherein R is an oligonucleotide bound to the compound by the 5′ of the oligonucleotide.
   139. The compound of any of clauses 100-138, wherein R is an oligonucleotide bound to the compound by the 3′ of the oligonucleotide.
   140. The compound of clause 100, wherein the compound is any of Formula (I-a), (I-b), (I-c), (I-d), (I-e), (I-f), (I-g), (I-h), (I-i), (I-j), (I-j1), (I-j2), (I-j-3), (I-k), (I-k-1), (I-k-2), (I-m), (I-m-1), and (I-m-2).
   141. A pharmaceutical composition comprising a compound of any of clauses 100-140, or a salt thereof, and optionally a pharmaceutically acceptable excipient.
   142. A method of treating a disease or condition in a subject in need thereof, the method comprising administering the compound of any of clauses 100-140, or a salt thereof, or the composition of claim 141.
   143. The method of clause 142, wherein the disease or condition is a muscle disease.
   144. A method of purifying a compound of Formula (II):

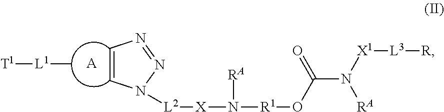

or a pharmaceutically acceptable salt, tautomer, stereoisomer, or isotopically enriched derivative thereof, wherein T¹, L¹, A, L², X, R^{A}, R¹, X¹, L³, and R are as defined in clause 1; the method comprising adding a volume of a solvent that is about 2, 3, 4, 5, 6, 7, 8, 9, or 10 times the amount of the total volume of a reaction mixture comprising the compound of Formula (II); and isolating the compound of Formula (II).
145. The method of clause 144, wherein the volume of solvent added is about 8 times the amount of the total volume of the reaction mixture.
146. The method of clause 144, wherein the volume of solvent added is about 3 times the amount of the total volume of the reaction mixture.
147. The method of any of clauses 144-146, wherein the solvent is acetone or isopropyl alcohol.
148. The method of any of clauses 144-147, wherein the solvent added in the purification is at a temperature that is equal to or below room temperature, 30° C., 25° C., 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C.
149. The method of any of clauses 144-148, wherein the solvent added in the purification is cooled to a temperature of about 0° C.
150. The method of any of clauses 144-149, further comprising adding, to the reaction mixture, a volume of an aqueous solution of a salt that is about 1, 0.9, 0.8, 0.7, 0.6, 0.5, 0.4, 0.3, 0.2, or 0.1 times the amount of the total volume of reaction mixture.
151. The method of any of clauses 144-150, further comprising adding, to the reaction mixture, a volume of a 3M aqueous solution of a salt that is about 0.1 times the amount of the total volume of the reaction mixture.
152. The method of clause 150 or 151, wherein the salt is NaCl.
153. The method of any of clauses 144-152, wherein purifying the compound of Formula (II) further comprises cooling the reaction mixture to a temperature that is equal to or below 20° C., 15° C., 10° C., 5° C., 0° C., −5° C., −10° C., −15° C., −20° C., −25° C., −30° C., −35° C., −40° C., −45° C., −50° C., −55° C., −60° C., −65° C., −70° C., −75° C., or −80° C.

1. A method of preparing a compound of Formula (I): 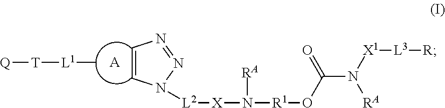
or a salt thereof, the method comprising coupling a targeting agent (Q) with a compound of Formula (II): 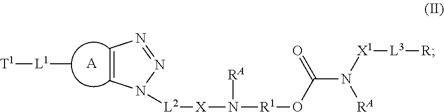
or a salt thereof, to provide a compound of Formula (I), wherein:
T is 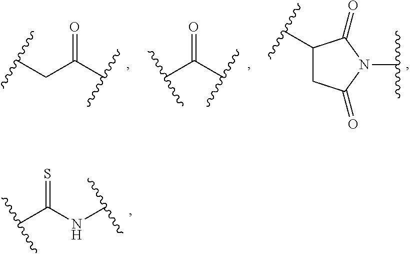
or —S—;
T¹ is 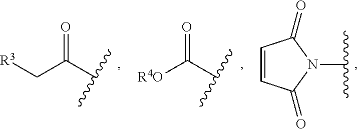
S═C═N—, or 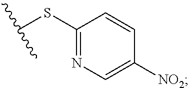 [SMILES: *Sc1ccc([N+](=O)[O-])cn1]
R³ is a leaving group;
R⁴ is hydrogen; substituted or unsubstituted alkyl; substituted or unsubstituted heterocyclyl; substituted or unsubstituted aryl; substituted or unsubstituted heteroaryl; or an oxygen protecting group;
L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
A is substituted or unsubstituted carbocycle or substituted or unsubstituted heterocycle, or A is absent and L¹ is bonded directly to the triazole ring;
L² is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
X is a cleavable moiety;
R¹ is substituted or unsubstituted arylene, or substituted or unsubstituted alkylene, or a combination thereof;
X¹ is a bond or a peptide;
L³ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
each R^{A} is independently hydrogen or substituted or unsubstituted alkyl; and
R is a molecular payload.

2. The method of claim 1, further comprising reacting a compound of Formula (III): 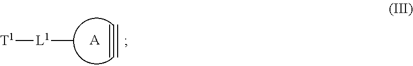
or a salt thereof, with a compound of Formula (IV): 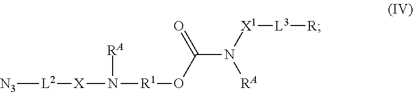
or a salt thereof, to provide a compound of Formula (II);
wherein A¹ is substituted or unsubstituted carbocycle or substituted or unsubstituted heterocycle, or A¹ is absent and L¹ is bonded directly to one terminus of the alkyne.

3. The method of claim 2, further comprising reacting a compound of Formula (V): 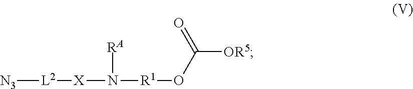
or a salt thereof, with a compound of Formula (VI): 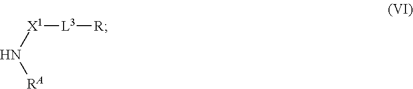
or a salt thereof, to provide a compound of Formula (IV), wherein:
R⁵ is substituted or unsubstituted aryl, or substituted or unsubstituted heteroaryl.

4. The method of claim 3, further comprising reacting a compound of Formula (VII): 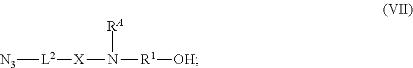
or a salt thereof, with a compound of Formula (VIII): 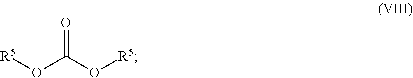
or a salt thereof, to provide a compound of Formula (V), wherein:
R⁵ is substituted or unsubstituted aryl, or substituted or unsubstituted heteroaryl.

5. The method of claim 4, further comprising reacting a compound of Formula (IX): 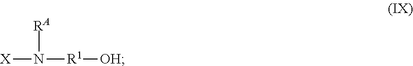
or a salt thereof, with a compound of Formula (X):
N₃-L²-LG (X);
or a salt thereof, to provide a compound of Formula (VII), wherein:
LG is a leaving group.

6. The method of claim 1, wherein the compound of Formula (I) is of Formula (I-c): 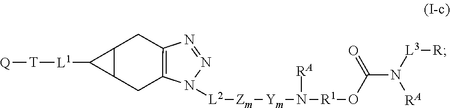
or a salt thereof, and the compound of Formula (II) is of Formula (I-c): 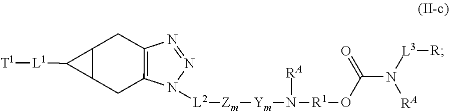
or a salt thereof, wherein:
each occurrence of Z is independently an amino acid;
each occurrence of Y is independently an amino acid; and
each m is independently 1, 2, or 3.

7. The method of claim 1, further comprising purifying the compound of Formula (II).

8. A method of preparing a compound of Formula (I): 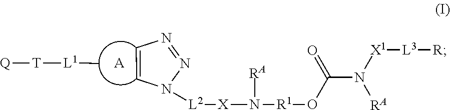
or a salt thereof, the method comprising reacting a compound of Formula (IV): 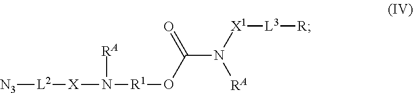
or a salt thereof, with a compound of Formula (B): 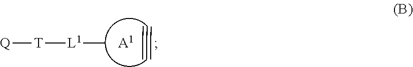
or a salt thereof, to provide a compound of Formula (I), wherein:
Q is targeting agent;
T is 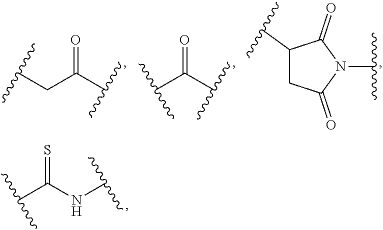
or —S—;
L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
A is substituted or unsubstituted carbocycle or substituted or unsubstituted heterocycle, or A is absent and L¹ is bonded directly to the triazole ring;
A¹ is substituted or unsubstituted carbocycle or substituted or unsubstituted heterocycle, or A¹ is absent and L¹ is bonded directly to one terminus of the alkyne;
L² is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
X is a cleavable moiety;
R¹ is substituted or unsubstituted arylene, or substituted or unsubstituted alkylene, or a combination thereof;
X¹ is a bond or a peptide;
L³ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
each R^{A} is independently hydrogen or substituted or unsubstituted alkyl; and
R is a molecular payload.

9. The method of claim 8, further comprising coupling a targeting agent (Q) with a compound of Formula (III): 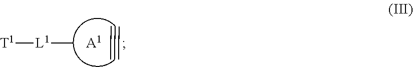
or a salt thereof, to provide a compound of Formula (B), wherein:
T is 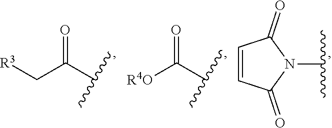
S═C═N—, or 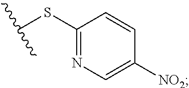 [SMILES: *Sc1ccc([N+](=O)[O-])cn1]
R³ is a leaving group; and
R⁴ is hydrogen; substituted or unsubstituted alkyl; substituted or unsubstituted heterocyclyl; substituted or unsubstituted aryl; substituted or unsubstituted heteroaryl; or an oxygen protecting group.

10. The method of claim 9, further comprising reacting a compound of Formula (V): 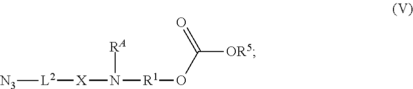
or a salt thereof, with a compound of Formula (VI): 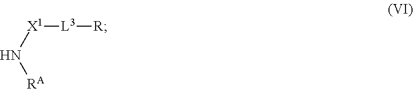
or a salt thereof, to provide a compound of Formula (IV), wherein:
R⁵ is substituted or unsubstituted aryl, or substituted or unsubstituted heteroaryl.

11. The method of any claim 10, further comprising reacting a compound of Formula (VII): 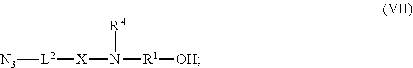
or a salt thereof, with a compound of Formula (VIII): 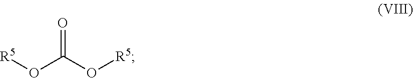
or a salt thereof, to provide a compound of Formula (V), wherein:
R⁵ is substituted or unsubstituted aryl, or substituted or unsubstituted heteroaryl.

12. The method of claim 11, further comprising reacting a compound of Formula (IX): 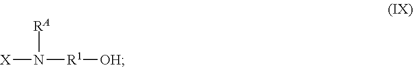
or a salt thereof, with a compound of Formula (X):
N₃-L²-LG (X);
or a salt thereof, to provide a compound of Formula (VII), or a salt thereof, wherein:
LG is a leaving group.

13. The method of claim 8, wherein the compound of Formula (I) is of Formula (I-c): 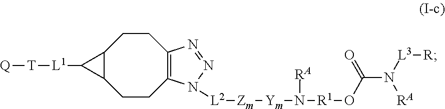
or a salt thereof, the compound of Formula (IV) is of Formula (IV-a): 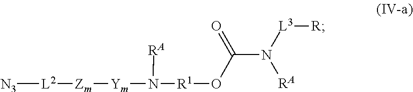
or a salt thereof, and the compound of Formula (B) is of Formula (B-a): 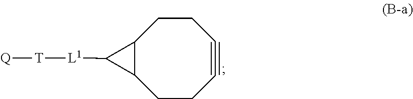
or a salt thereof, wherein:
each occurrence of Z is independently an amino acid;
each occurrence of Y is independently an amino acid; and
each m is independently 1, 2, or 3.

14. A method of preparing a compound of Formula (I): 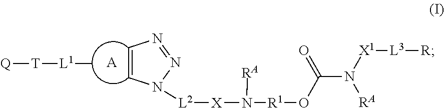
or a salt thereof, the method comprising reacting a targeting agent (Q); a compound of Formula (IV): 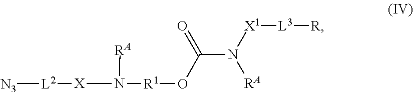
or a salt thereof; and a compound of Formula (III): 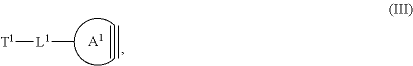
or a salt thereof; to provide a compound of Formula (I), or a salt thereof, wherein:
T is 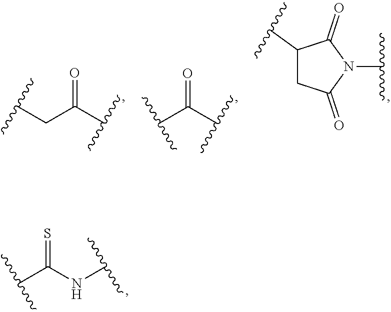
or —S—;
T¹ is 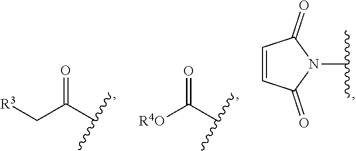
S═C═N—, or 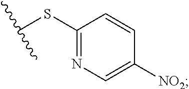 [SMILES: *Sc1ccc([N+](=O)[O-])cn1]
R³ is a leaving group (e.g., halogen, tosylate, mesylate, or triflate);
R⁴ is hydrogen; substituted or unsubstituted alkyl; substituted or unsubstituted heterocyclyl; substituted or unsubstituted aryl; substituted or unsubstituted heteroaryl; or an oxygen protecting group;
L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
A is substituted or unsubstituted carbocycle or substituted or unsubstituted heterocycle, or A is absent and L¹ is bonded directly to the triazole ring;
A¹ is substituted or unsubstituted carbocycle or substituted or unsubstituted heterocycle, or A¹ is absent and L¹ is bonded directly to one terminus of the alkyne;
L² is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
X is a cleavable moiety;
R¹ is substituted or unsubstituted arylene, or substituted or unsubstituted alkylene, or a combination thereof;
X¹ is a bond or a peptide;
L³ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
each R^{A} is independently hydrogen or substituted or unsubstituted alkyl; and
R is a molecular payload.

15. The method of claim 14, wherein the compound of Formula (I) is of Formula (I-c): 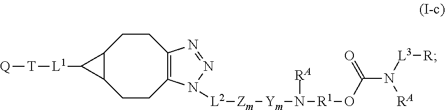
or a salt thereof, the compound of Formula (IV) is of Formula (IV-b): 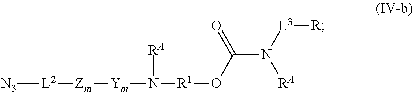
or a salt thereof, and the compound of Formula (III) is of Formula (III-a): 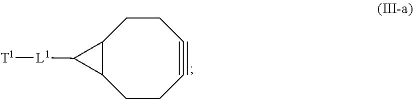
or a salt thereof, wherein:
each occurrence of Z is independently an amino acid;
each occurrence of Y is independently an amino acid; and
each m is independently 1, 2, or 3.

16. The method of claim 1, wherein
Q is an antibody; and R is an oligonucleotide.

17. The method of claim 1, wherein
Q is an anti-TfR antibody; and R is a phosphorodiamidate morpholino oligomer (PMO).

18. A compound of Formula (I): 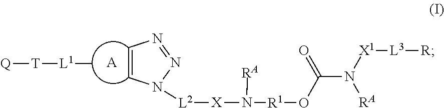
or a pharmaceutically acceptable salt, tautomer, stereoisomer, or isotopically enriched derivative thereof, wherein:
Q is a targeting agent;
T is 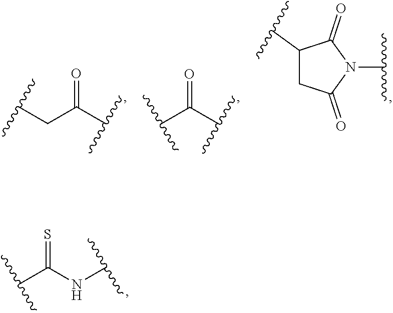
or —S—;
L¹ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
A is substituted or unsubstituted carbocycle or substituted or unsubstituted heterocycle, or A is absent and L¹ is bonded directly to the triazole ring;
L² is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
X is a cleavable moiety;
R¹ is substituted or unsubstituted arylene, or substituted or unsubstituted alkylene, or a combination thereof;
X¹ is a bond or a peptide;
L³ is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof;
each R^{A} is independently hydrogen or substituted or unsubstituted alkyl; and
R is a molecular payload.

19. The compound of claim 18, wherein the compound is of Formula (I-c): 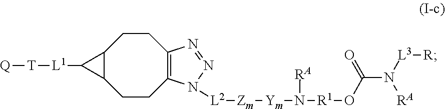
or a salt thereof, wherein:
each occurrence of Z is independently an amino acid;
each occurrence of Y is independently an amino acid; and
each m is independently 1, 2, or 3.

20. The compound of claim 18, wherein:
Q is an antibody; and R is an oligonucleotide.

21. (canceled)
